(19) United States
(12) Patent Application Publication
(10) Pub. No.: US 2010/0291611 A1
(43) Pub. Date: Nov. 18, 2010

(54) Assays

(75) Inventors: John Anthony Bolbot, Clackmannanshire (GB); John William Dilleen, Clackmannanshire (GB); Christopher John Slevin, Edinburgh (GB)

Correspondence Address: STEPTOE & JOHNSON LLP, 1330 CONNECTICUT AVENUE, N.W., WASHINGTON, DC, 20036, US

(73) Assignee: Inverness Medical Switzerland GMBH, Zug (CH)

(21) Appl. No.: 12/278,883

(22) PCT Filed: Feb. 15, 2007
(86) PCT No.: PCT/IB07/00369

§ 371 Date: Jul. 21, 2010

Related U.S. Application Data
(60) Provisional application No. 60/885,320, filed on Jan. 17, 2007.
(60) Provisional application No. 60/889,962, filed on Feb. 15, 2007.

(30) Foreign Application Priority Data
Feb. 15, 2006 (GB) 0603049.8

(51) Int. Cl. C12Q 1/02; C12M 1/00; G01N 33/48; G01N 33/20

(52) U.S. Cl. 435/29; 435/287.1; 422/68.1; 436/84; 422/82.01

(57) ABSTRACT

A device includes a substrate that defines, at least in part, a microfluidic network including an inlet in communication with a first detection zone and with a second detection zone. A cobalt reagent and a nickel reagent are disposed within the microfluidic network. First electrodes are in communication with the first detection zone and second electrodes arc in communication with the second detection zone. The device is configured to receive a blood derived sample introduced to the inlet, partition the blood sample into first and second blood sample portions, form a first mixture including at least some of the first blood sample portion and at least some of the cobalt reagent, and form a second mixture including at least some of the second blood sample portion, at least some of the cobalt reagent and at least some of the nickel reagent.

### CLAIM OF PRIORITY

This application claims priority to U.S. Provisional Application No. 60/889,962, filed Feb. 15, 2007, U.S. Provisional Application No. 60/885,320, filed Jan. 17, 2007, and GB Provisional Application No. GB 0603049.8, filed Feb. 15, 2006, each of which is incorporated by reference in its entirety.

### TECHNICAL FIELD

The present invention relates to assays (e.g., for the determination of the presence of an ischemic event in an animal such as a human).

### BACKGROUND

Assays for species of interest (e.g., analytes) have many applications (e.g., in medicine, industry, and environmental analysis). Typically, analytes are present in sample materials (e.g., mixtures) that include one or more concomitant species. For example, albumin, a mammalian blood protein, is an exemplary analyte. Albumin is found in blood, a sample material that includes concomitant species such as particulates (e.g., macrophages and red blood cells), ionic species (e.g., various salts and metal ions), gases (e.g., solvated oxygen and nitrogen), and multiple biological compounds (e.g., proteins, lipoproteins, blood triglycerides, fatty acids and cholesterol). The amount and/or type of concomitant species as well as other sample properties (e.g., pH, temperature, and viscosity) can vary with the type of sample material and between different samples of the same sample material. Concomitant species and variations in sample properties can interfere with (e.g., reduce the accuracy and/or precision) of assays. Such interferences are known as matrix effects.

Assays have been used to determine the presence of ischemia, a condition associated with poor oxygen supply to a part of the body due to, for example, a constriction or an obstruction of a blood vessel. Two common forms of ischemia are cardiovascular ischemia and cerebral ischemia. The former is generally a direct consequence of coronary artery disease, while the latter is often due to a narrowing of the arteries leading to the brain. When a subject experiences an ischemic event, ischemia modified albumin (IMA) appears in the subject's blood and the amount of normal (i.e., unmodified) albumin in the subject's blood decreases.

One difference between IMA and normal albumin is that IMA has a lower capacity to bind certain metal ions. International Patent Publication WO 03/046538 describes electrochemical methods and devices for in vitro detection of an ischemic event in a patient sample based on this difference. The publication describes adding a known amount of a transition metal ion to a blood sample and then measuring the current or potential difference of metal ion that is free of normal albumin (e.g., metal ion that is unsequestered or unbound by normal albumin). Because the presence of an ischemic event results in decreased amounts of normal albumin, the amount of free metal ion is higher in the presence of an ischemic event than in the absence of an ischemic event. The WO 03/046538 publication is incorporated herein by reference in its entirety.

The U.S. Food and Drug Administration approved an Albumin Cobalt Binding (ACB®) Test for use in the determination of an ischemic event. The test works by optically measuring how much cobalt is bound to the blood protein albumin. Serum from a subject is combined with a reagent that forms a colored complex with free cobalt but not with cobalt that is complexed with albumin. The amount of colored complex is determined optically and compared to a standard value to diagnose the presence of an ischemic event in the subject.

### SUMMARY

The present invention relates to assays (e.g., for the determination of the presence of an ischemic event in a mammal such as a human).

Exemplary embodiments of assay methods, systems and devices include the following, and all combinations thereof:
1. An assay device comprising:
   means to form a first mixture comprising a cobalt reagent and a blood derived sample material,
   means to determine an amount or concentration of free cobalt in the first mixture,
   means to form a second mixture comprising a cobalt reagent, blood derived sample material, and optionally a nickel reagent,
   wherein if no nickel reagent is contained in the second mixture, then the amount of cobalt in the second mixture is different from the amount of cobalt in the first mixture, and
   means to determine an amount or concentration of free cobalt in the second mixture.
2. The assay device of embodiment 1, wherein the means to form a second mixture comprises a nickel reagent.
3. The assay device of embodiment 2, wherein the nickel reagent disposed in a dry state within the device.
4. The assay device of any of embodiment 1 to 3, wherein the means to form a first mixture comprises a cobalt reagent disposed in a dry state within the device.
5. The assay device of any of the preceding embodiments, wherein the first and second mixtures further comprise a chloride compensating agent in an amount sufficient to raise the total chloride concentration to at least 50 mM.
6. The assay device of any of the preceding embodiments, wherein the device comprises first and second detection zones in communication with a common sample receiving zone.
7. An assay device comprising:

a first detection zone and a second detection zone,

a first reagent material comprising a first metal, and

a second reagent material comprising either the first metal and a second metal having a higher affinity for albumin than the first metal, or comprising a different amount of the first metal;

wherein:

the device is configured to receive sample liquid and form a first mixture in the first detection zone, the first mixture comprising a portion of the sample liquid and the first reagent material, and to form a second mixture in the second detection zone, the second mixture comprising a portion of the sample liquid and the second reagent material.
8. The assay device according to embodiment 5, wherein said first and second detection zones are electrochemical detection zones.
9. The assay device according to embodiment 5 or 6, wherein said first metal is cobalt.
10. The assay device according to any of embodiments 5-7, wherein said second metal is nickel.
11. The device of any of embodiments 5 to 8, wherein the first metal is selected from the group consisting of V, As, Co, Cu, Sb, Cr, Mo, Mn, Ba, Zn, Ni, Hg, Cd, Fe, Pb, Au and Ag.
12. The method of embodiment 5 to 9, wherein the second metal is selected from the group consisting of V, As, Co, Cu, Sb, Cr, Mo, Mn, Ba, Zn, Ni, Hg, Cd, Fe, Pb, Au and Ag.
13. The assay device according to any of embodiments 5-10, wherein at least one of said first and said second metal is present in the dry state before application of sample liquid.
14. The assay device according to any of embodiments 5-11, wherein said sample liquid is selected from human blood and human blood plasma.
15. The assay device according to any of embodiments 5-12, wherein the first or second metal is in the form of a salt of the metal.
16. An assay system comprising an assay reader and the assay device of any of the previous embodiments.
17. The assay system of embodiment 16, wherein the assay reader is configured to operate the assay device to determine a respective amount of free first metal in the first and second detection zones.
18. An assay reader configured to receive an assay device according to any of embodiments 1-15.
19. The assay reader of embodiment 18, wherein the reader comprises at least one electrochemical detector.
20. The assay reader of embodiment 18 or 19 comprising an assay device lock mechanism suitable to lock and unlock the assay device according to any of embodiments 1-15.
21. The assay reader of embodiment 20, wherein the lock mechanism comprises a movable member configured to engage a recess of the assay device upon insertion of the assay device into the assay reader.
22. The assay reader of embodiment 21, where the lock mechanism comprises two movable members each configured to engage a respective recess of the assay device upon insertion of the assay device into the assay reader.
23. The assay reader of embodiment 21 or 22, wherein the movable member(s) is/are rotatable.
24. An assay reader according to any of embodiments 18-23 further comprising an assay device temperature controller.
25. The assay reader of embodiment 24, wherein the temperature controller comprises a movable thermal block configured to move from a resting position to a thermally engaged position upon insertion of an assay device into the assay reader, wherein, in the thermally engaged position, the heating element is disposed in thermal contact with the assay device.
26. The use of an assay device according to any of embodiments 1-15, an assay system according to any of embodiments 16 or 17, or an assay reader according to any of embodiments 18-23 to determine the amount or concentration of free cobalt or metal of the first metal reagent in a blood derived sample material.
27. The use according to embodiment 26, wherein the amount or concentration of free cobalt or metal of the first metal reagent in a blood derived sample material is used to determine the presence of an ischemic event in a mammal from which said blood derived sample material was taken.
28. The use of an assay device according to any of embodiments 1-15, an assay system according to any of embodiments 16 or 17, or an assay reader according to any of embodiments 18-23 to determine the amount or concentration of ischemia-modified albumin (IMA) in a blood derived sample material.
29. The use according to embodiment 28 wherein the concentration or amount of ischemia-modified albumin (IMA) is used to determine the presence of an ischemic event in a mammal from which said blood derived sample material was taken.
30. The use of an assay device according to any of embodiments 1-15, an assay system according to any of embodiments 16 or 17, or an assay reader according to any of embodiments 18-23 to determine the presence of an ischemic event in a mammal from which said blood derived sample material was taken.
31. A method for determining the presence of ischemia modified albumin in a sample derived from human blood, comprising

forming a first mixture adding a part of said blood derived sample material to a cobalt reagent

determining an amount or concentration of free cobalt in the first mixture to obtain a first result,

forming a second mixture by adding another part of said blood derived sample material to a cobalt reagent and optionally an amount of a nickel reagent sufficient to substantially prevent the formation and presence of cobalt albumin complexes in said part of the blood derived sample material,

wherein if no nickel reagent is contained in the second mixture, then the amount of cobalt in the second mixture is different from the amount of cobalt in the first mixture,

determining an amount or concentration of free cobalt in the second mixture to obtain a second result

processing the first and the second results and comparing the processed value with a suitable reference value that is indicative of ischemia-modified albumin.
32. The method according to embodiment 31 wherein the reference value is indicative of the amount or concentration of free cobalt in a sample derived from blood of a human known to contain ischemic modified albumin following an ischemic event.
33. The method according to embodiment 31 wherein the reference value is indicative of the amount or concentration of free cobalt in a sample derived from blood of the same human taken earlier.
34. The method according to any of embodiments 31-34 using an assay device according to any of embodiments 1-15, an assay system according to any of embodiments 16 or 17, or an assay reader according to any of embodiments 18-23.
35. A method for determining the presence of ischemia modified albumin in a sample derived from human blood, comprising

forming a first mixture adding a part of said blood derived sample material to a first reagent comprising a first metal,

determining an amount or concentration of free first metal in the first mixture to obtain a first result,

forming a second mixture by adding another part of said blood derived sample material to an amount of the first reagent and optionally an amount of a second reagent comprising a second metal having a higher affinity for albumin than the first metal, the amount of second metal being sufficient to substantially prevent the formation and presence of first metal-albumin complexes in said part of the blood derived sample material,

wherein if no second reagent is contained in the second mixture, then the amount of first metal in the second mixture is different from the amount of first metal in the first mixture,

determining an amount or concentration of free first metal in the second mixture to obtain a second result

processing the first and the second results and comparing the processed value with a suitable reference value that is indicative of ischemia-modified albumin.
36. The method of embodiment 17, wherein the first metal is selected from the group consisting of V, As, Co, Cu, Sb, Cr, Mo, Mn, Ba, Zn, Ni, Hg, Cd, Fe, Pb, Au and Ag.
37. The method of embodiment 18, wherein the second metal is selected from the group consisting of V, As, Co, Cu, Sb, Cr, Mo, Mn, Ba, Zn, Ni, Hg, Cd, Fe, Pb, Au and Ag.
38. The method according to any of embodiments 17-18 using an assay device according to any of embodiments 1-15, an assay system according to any of embodiments 16 or 17, or an assay reader according to any of embodiments 18-23.
39. An assay system, comprising:

an assay device comprising first and second electrochemical detection zones each comprising a mixture of blood derived sample material and a cobalt reagent, the sample material having been obtained from a mammal, and

an assay reader configured to operate the assay device to determine a respective amount of free cobalt in the first and second detection zones and determine the presence of an ischemic event in the mammal based on the amounts of free cobalt.
40. A method, comprising:
   forming a first mixture comprising cobalt and first blood-derived sample material derived from blood of a mammal, the sample material comprising albumin,
   obtaining a first signal indicative of an amount of cobalt not complexed with albumin present in the first mixture,
   forming a second mixture comprising cobalt, nickel, and second blood-derived sample material derived from blood of the mammal,
   obtaining a second signal indicative of an amount of cobalt not complexed with albumin present in the second mixture, and
   based on at least the first and second signals, determining an assay result indicative of the presence of ischemia in the mammal.
41. The method of embodiment 40, further comprising determining the presence of ischemia in the mammal based at least in part on the assay result.
42. The method of embodiments 40 or 41, wherein the mammal is a human.
43. The method of any of the previous embodiments, wherein the assay result is indicative of a difference between the first and second electrochemical signals, a ratio of the first and second electrochemical signals, or a combination thereof.
44. The method of any of the previous embodiments, wherein the cobalt of the first and second mixtures is cobalt ion and the nickel of the second mixture is nickel ion.
45. The method of embodiment 40, wherein the first signal is a first electrochemical signal and the second signal is a second electrochemical signal.
46. The method of embodiment 45, wherein obtaining the first electrochemical signal is performed amperometrically and obtaining the second electrochemical signal is performed amperometrically.
47. The method of embodiment any of the previous embodiments, wherein forming the first mixture comprises combining the first sample material and a cobalt salt, the cobalt salt being in a dry state prior to forming the first mixture.
48. The method of embodiment 47, wherein forming the second mixture comprises combining the second sample material, a cobalt salt, and a nickel salt, the cobalt and nickel salts being in a dry state prior to forming the second mixture.
49. The method of any of the previous embodiments, wherein the first mixture comprises a first total amount of chloride ion resulting from a cobalt salt, the second mixture comprises a second total amount of chloride ion resulting from a nickel salt and a cobalt salt, and the first mixture comprises an amount of chloride resulting from a cobalt- and nickel-free source of chloride, the amount of chloride being at least 50% of the difference between the second and first total amounts of chloride.
50. The method of embodiment 49, wherein the cobalt- and nickel-free source of chloride comprises an alkali metal chloride salt.
51. The method of embodiment 50, wherein substantially all of the alkali metal salt is potassium chloride.
52. The method of embodiment 51, wherein the amount of chloride is at least 75% of the difference between the second and first total amounts of chloride.
53. The method of any of embodiments 47-52, wherein, prior to forming the first mixture, the cobalt salt is in a dry state within a microfluidic device and forming the first mixture comprises combining blood-derived sample material of the mammal with the cobalt salt within the microfluidic device.
54. The method of embodiment 53, wherein combining the blood-derived sample material of the mammal with the cobalt salt comprises mechanically moving the blood-derived sample material within the microfluidic device.
55. The method of embodiment 54, wherein mechanically moving blood-derived sample material within the microfluidic device comprises magnetically moving a magnetically susceptible member disposed in contact with the blood-derived sample material and the cobalt salt within the microfluidic device.
56. The method of embodiment 55, comprising inducing rotary movement of the member.
57. The method of embodiment 53, wherein forming the second mixture comprises combining the second sample material, a nickel salt, and a cobalt salt, the nickel and cobalt salts being in a dry state prior to performing the step of combining.
58. The method of embodiment 57, wherein, prior to forming the second mixture, the nickel and cobalt salts are in a dry state within the microfluidic device and forming the second mixture comprises combining blood-derived sample material of the mammal and the nickel and cobalt salts within the microfluidic device.
59. The method of embodiment 58, wherein combining the blood-derived sample material of the mammal with the nickel and cobalt salts of the second mixture comprises mechanically moving blood-derived sample material within the microfluidic device and combining the blood-derived sample material of the mammal with the cobalt salts of the first mixture comprises mechanically moving blood-derived sample material within the microfluidic device.
60. The method of embodiment 59, wherein mechanically moving the blood-derived sample material comprises magnetically moving a magnetically susceptible member disposed within the microfluidic device.
61. The method of any of the previous embodiments, comprising receiving an amount of blood-derived sample material from the mammal within a microfluidic device, partitioning the blood-derived sample material into at least first and second portions, and wherein the first sample comprises blood-derived sample material of the first portion of blood-derived sample material and the second sample comprises blood-derived sample material of the second portion of blood-derived sample material.
62. The method of embodiment 61, comprising, after partitioning the blood-derived sample material, passing the first portion of blood along a channel of the microfluidic device to an electrochemical detection chamber of the microfluidic device and passing the second portion of blood-derived sample material along a channel of the microfluidic device to an electrochemical detection chamber of the microfluidic device.
63. The method of embodiment 62, wherein the step of forming the first mixture is performed in the first detection chamber and the step of forming the second mixture is performed in the second detection chamber.
64. The method of embodiment 63, wherein the first signal is a first electrochemical signal obtained using electrodes of the first detection chamber and the second signal is a second electrochemical signal obtained using electrodes of the second detection chamber.
65. The method of embodiment 62, wherein passing the first portion of blood-derived sample material along the channel comprises passing the blood-derived sample material from a channel portion having a first cross sectional area to a channel portion having a second, smaller cross sectional area prior to passing the blood-derived sample material into the detection chamber.
66. A method, comprising:
   forming a first mixture comprising a first reagent that forms a complex with albumin and a first blood-derived sample material derived from blood of a mammal,
   determining a first value indicative of an amount of first reagent not complexed with albumin present in the first mixture,
   forming a second mixture comprising the first reagent, a second reagent that competes with the first reagent for forming a complex with albumin, and a second sample, the second sample being derived from blood-derived sample material of the mammal,
   determining a second value indicative of an amount of first reagent not complexed with albumin present in the second mixture, and
   based on at least the first and second values, determining a value indicative of an amount of albumin present in the first mixture.
67. The method of embodiment 66, wherein the first reagent is cobalt.
68. The method of embodiment 66 or 67, wherein the second reagent is nickel.
69. A device, comprising:

a substrate that defines, at least in part, a microfluidic network comprising an inlet in communication with a first detection zone and with a second detection zone,

cobalt salt disposed within the microfluidic network,

nickel salt disposed within the microfluidic network,

first electrodes in electrochemical communication with the first detection zone, and

second electrodes in electrochemical communication with the second detection zone,

wherein:

the device is configured to receive a blood derived sample material introduced to the inlet, partition the sample material into first and second blood sample portions, form a first mixture comprising at least some of the first sample, material portion and at least some of the cobalt salt, and form a second mixture comprising at least some of the second sample material portion, at least some of the cobalt salt and at least some of the nickel salt.
70. The device of embodiment 69, wherein the first and second electrodes are each configured to permit determination of cobalt present in the first and second mixtures.
71. The device of embodiment 69 or 70, wherein a maximum volume of each of the first and second detection zones is 25 μl.
72. The device of any of embodiments 69 to 71, wherein the device is configured to form a minimum cobalt concentration in the first mixture of 500 μM.
73. The device of any of embodiments 69 to 72, wherein the device is configured to form a maximum cobalt concentration in the first mixture of 10 mM.
74. The device of any of embodiments 69 to 73, wherein the device is configured to form a maximum nickel concentration in the first mixture of 1 mM.
75. The device of any of embodiments 69 to 74, wherein the device is configured to form a minimum nickel concentration in the second mixture of 4 mM.
76. The device of any of embodiments 69 to 75, wherein the device is configured to form a maximum nickel concentration in the second mixture of 25 mM.
77. The device of any of embodiments 69 to 76, wherein the device is configured to form a minimum cobalt concentration in the second mixture of 500 μM.
78. The device of any of embodiments 69 to 77, wherein the device is configured to form a maximum cobalt concentration in the second mixture of 10 mM.
79. The device of any of embodiments 69 to 78, wherein, prior to use, the cobalt salt is disposed within the microfluidic device in a dry state and the nickel salt is disposed within the microfluidic device in a dry state.
80. The device of embodiment 79, wherein at least some of the dry cobalt reagent is disposed in each of the first and second detection zones.
81. An electrochemical device, comprising:

a substrate that defines, at least in part, a microfluidic network comprising an inlet in communication with a first detection zone,

a substrate that defines, at least in part, a microfluidic network comprising an inlet in communication with a second detection zone,

dry cobalt salt disposed in communication with the microfluidic network comprising the first detection zone,

dry cobalt salt in communication with the microfluidic network comprising the second detection zone,

dry nickel salt disposed within the microfluidic network comprising the second detection zone,

first electrodes in electrochemical communication with the first detection zone, and

second electrodes in electrochemical communication with the second detection zone,

wherein:

the device is configured to receive a blood-derived sample material introduced to the inlet in communication with the first detection zone, form a first mixture comprising at least some of the blood-derived sample material and at least some of the cobalt salt of the microfluidic network comprising the first detection zone, and form a second mixture comprising at least some of the blood-derived sample material, at least some of the cobalt salt of the microfluidic network comprising the second detection zone, and at least some of the nickel salt.
82. The electrochemical device of embodiment 81, wherein the substrate that defines, at least in part, the microfluidic network comprising the inlet in communication with the first detection zone and the substrate that defines, at least in part, the microfluidic network comprising the inlet in communication with the second detection zone are the same substrate.
83. The electrochemical device of embodiment 81 or 82, wherein the inlet of the microfluidic network comprising the first detection zone and the inlet of the microfluidic device comprising the second detection zone is the same inlet.
84. An electrochemical device, comprising:

a substrate that defines, at least in part, a microfluidic network comprising an inlet in communication with a first detection zone,

a substrate that defines, at least in part, a microfluidic network comprising an inlet in communication with a second detection zone,

a first reagent disposed within the microfluidic network comprising the first detection zone, the first reagent being capable of forming a complex with albumin,

first reagent disposed within the microfluidic network comprising the second detection zone,

a second reagent disposed within the microfluidic network comprising the second detection zone, the second reagent being capable of competing with the first reagent to form a complex with albumin,

first electrodes in electrochemical communication with the first detection zone, and

second electrodes in electrochemical communication with the second detection zone,

wherein:

the device is configured to receive a blood-derived sample material introduced to the inlet in communication with the first detection zone, form a first mixture comprising at least some of the blood-derived sample material and at least some of the first reagent of the microfluidic network comprising the first detection zone, and form a second mixture comprising at least some of the blood-derived sample material, at least some of the first reagent of the microfluidic network comprising the second detection zone, and at least some of the second reagent.
85. A device, comprising:

a substrate that defines, at least in part, a microfluidic network comprising an inlet, a first detection zone in communication with the junction and a second detection zone in communication with the junction,

a first reagent disposed in a dry state within the microfluidic network, the first reagent capable, when mobilized by a blood derived sample, of forming a complex comprising albumin,

a second reagent disposed in a dry state within the microfluidic network, the second reagent capable, when mobilized by a blood-derived sample material, of competing with the first reagent to form a complex comprising albumin,

first electrodes in electrochemical communication with the first detection zone, and

second electrodes in electrochemical communication with the second detection zone,

wherein:

the device is configured to receive a blood-derived sample material introduced to the inlet, partition the blood sample at the junction into first and second blood-derived sample material portions, form a first mixture comprising at least some of the first blood-derived sample material portion and at least some of the first reagent, and form a second mixture comprising at least some of the second blood-derived sample material portion, at least some of the first reagent and at least some of the second reagent.
86. A method, comprising:

receiving blood-derived sample material of a mammal within a microfluidic device,

introducing at least some of the blood-derived sample material into a detection zone of the microfluidic device, the detection zone comprising an amount of dry cobalt salt and electrodes,

contacting, with a first surface of the blood-derived sample material, substantially all of the dry cobalt salt present in the detection zone,

contacting, with a second surface of the blood-derived sample material, the electrodes, the second surface being opposed to the first surface,

combining the blood-derived sample material and cobalt salt to form a mixture, and

operating the electrodes to determine an amount of cobalt not complexed with albumin present in the mixture.
87. The method of embodiment 86, wherein the substantially all of the dry cobalt salt is at least 70% by weight of the dry cobalt salt present in the detection zone.
88. The method of embodiment 86 or 87, wherein the substantially all of the dry cobalt salt is disposed on the first major surface, the step of introducing comprises introducing the blood-derived sample material from a channel of the microfluidic device into the detection zone, and the step of contacting, with a first surface of the blood-derived sample material, comprises solubilizing dry cobalt disposed on the first major surface.
89. The method of any of embodiments 86 to 88, wherein the step of operating is performed with the electrodes positioned beneath the first surface.
90. A method, comprising:

receiving blood-derived sample material of a mammal within a microfluidic device,

introducing at least some of the blood-derived sample material into a detection zone of the microfluidic device, the detection zone comprising an amount of a reagent and electrodes, the reagent being capable of forming a complex with albumin present in the blood-derived sample material,

contacting, with a first surface of the blood-derived sample material, substantially all of the reagent present in the detection zone,

contacting, with a second surface of the blood-derived sample material, the electrodes, the second surface being opposed to the first surface,

combining the blood-derived sample material and reagent to form a mixture, and

operating the electrodes to determine an amount of reagent not complexed with albumin present in the mixture.
91. A device, comprising:

a substrate that defines, at least in part, a microfluidic network comprising an inlet and a detection zone in communication therewith, the detection zone having first and second opposed major surfaces,

a cobalt salt disposed in a dry state within the detection zone, and

electrodes disposed on the first major surface of the detection zone,

wherein:

prior to use, essentially none of the cobalt salt is disposed on the first major surface of the detection zone, and

in use, the device is configured to receive a blood-derived sample material introduced to the inlet and, within the detection zone, form a mixture comprising at least some of the blood-derived sample material and at least some of the cobalt.
92. The device of embodiment 91, further comprising a processor in communication with the electrodes, wherein, in use, the processor is configured to operate the electrodes to determine an amount of cobalt not complexed with albumin present in the mixture formed in the detection zone.
93. A device, comprising:

a substrate that defines, at least in part, a microfluidic network comprising an inlet and a detection zone in communication therewith, the detection zone having first and second opposed major surfaces,

a reagent disposed in a dry state within the detection zone, the reagent capable, when mobilized by a blood-derived sample material obtained from a mammal, of forming a complex comprising albumin, and

electrodes disposed on the first major surface of the detection zone,

wherein:

prior to use, essentially none of the reagent is disposed on the first major surface of the detection zone, and

in use, the device is configured to receive a blood-derived sample material introduced to the inlet and, within the detection zone, form a mixture comprising at least some of the blood-derived sample material and at least some of the reagent.
94. The device of embodiment 93, wherein, prior to use, less than 25% of the total mass of the reagent present in the detection zone is disposed on the first major surface of the detection zone.
95. The device of embodiment 93 or 94, wherein, prior to use, less than 10% of the total mass of the reagent present in the detection zone is disposed on the first major surface of the detection zone.
96. The device of any of embodiments 93 to 95, further comprising a processor in communication with the electrodes, wherein, in use, the processor is configured to operate the electrodes to determine an amount of reagent not complexes with cobalt present in the mixture formed in the detection zone.
97. The device of any of embodiments 93 to 96, wherein the mammal is a human and the processor is configured to determine the presence of an ischemic state in the human based at least in part on the amount of uncomplexed reagent present in the mixture.
98. The device of any of embodiments 93 to 97, wherein the reagent is a cobalt salt and it is the cobalt of the cobalt salt that is capable, when mobilized by a blood derived sample, of forming a complex comprising albumin.
99. The device of any of embodiments 93 to 98, wherein the detection zone is a first detection zone, the reagent is a first reagent, the electrodes are first electrodes, and the device further comprises a second detection zone in communication with an inlet, the to second detection zone having first and second opposed major surfaces,

an amount of the first reagent disposed in a dry state disposed within the second detection zone,

a second reagent disposed in a dry state within the second detection zone, the second reagent capable, when mobilized by a blood derived sample obtained from a mammal, of competing with the first reagent to form a complex comprising albumin and the second reagent but excluding the first reagent, and

second electrodes disposed on the first major surface of the second detection zone,

wherein:

prior to use, essentially none of the first and second reagents are disposed on the first major surface of the second detection zone, and

in use, the device is configured to receive a blood derived sample introduced to the inlet and, within the second detection zone, form a second mixture comprising at least some of the blood sample and at least some of the first and second reagents.
100. The device of embodiment 99, further comprising a processor in communication with the first and second electrodes, wherein, in use, the processor is configured to operate the first and second electrodes to determine an amount of uncomplexed first reagent present in the respective mixtures formed in the first and second detection zones.
101. The device of embodiment 99 or 100, wherein the mammal is a human and the processor is configured to determine the presence of an ischemic state in the human based at least in part on the amount of uncomplexed first reagent present in the first and second mixtures.
102. The device of any of embodiments 99 to 101, wherein the first reagent is a cobalt salt and it is the cobalt of the cobalt salt that is capable, when mobilized by a blood derived sample, of forming a complex comprising albumin and the second reagent is a nickel salt, and it is the nickel of the nickel salt that is capable, when mobilized by a blood derived sample, of competing with cobalt to form a complex comprising albumin and nickel but excluding cobalt.
103. The device of any of embodiments 99 to 102, wherein the detection zone comprises a member capable of mechanically moving blood-derived sample material within the microfluidic device.
104. The method of embodiment 103, wherein the member is a magnetically susceptible member and the device further comprises a magnetically susceptible field source capable of moving the member within the detection zone.
105. A method, comprising:

receiving blood-derived sample material of a mammal within a microfluidic device,

combining, within the microfluidic device, a first portion of the blood-derived sample material with dry cobalt salt to form a first mixture,

combining, within the microfluidic device, a second portion of the received blood-derived sample material with dry cobalt salt and dry nickel salt to form a second mixture,

with the first mixture disposed in a first detection zone of the device, determining a value indicative of the amount of cobalt present in the first mixture,

with the second mixture disposed in a second detection zone of the device, determining a value indicative of the amount of cobalt present in the second mixture;

wherein:

the steps of combining each comprise magnetically moving a respective magnetically susceptible member each disposed within a different spaced apart location of the microfluidic device.
106. A method, comprising:

receiving blood-derived sample material of a mammal within a microfluidic device,

combining, within the microfluidic device, a first portion of the blood-derived sample material with a first reagent to form a first mixture, the first reagent being capable of forming a complex with albumin,

combining, within the microfluidic device, a second portion of the received blood-derived sample material with an amount of the first reagent and a second reagent to form a second mixture, the second reagent being capable of competing with the first reagent to form a complex with albumin that excludes the first reagent,

with the first mixture disposed in a first detection zone of the device, determining a value indicative of the amount of first reagent present in the first mixture,

with the second mixture disposed in a second detection zone of the device, determining a value indicative of the amount of first reagent present in the second mixture;

wherein:

the steps of combining each comprise magnetically moving a respective magnetically susceptible member each disposed within a different spaced apart location of the microfluidic device.
107. A device, comprising:

a substrate that defines, at least in part, a microfluidic network comprising an inlet, a first detection zone in communication with the junction, and a second detection zone in communication with the junction,

a first reagent disposed in a dry state within the first detection zone,

a first magnetically susceptible member disposed in the first detection zone,

an amount of the first reagent disposed in a dry state within the second detection zone,

a second magnetically susceptible member disposed in the second detection zone,

first electrodes in electrochemical communication with the first detection zone, and

second electrodes in electrochemical communication with the second detection zone,

a magnetic field system configured to magnetically manipulate the first and second magnetically susceptible members,

a processor configured to actuate the magnetic field system,

wherein:

the device is configured to receive a blood derived sample introduced to the inlet, partition the blood sample at the junction into first and second blood-derived sample material portions, receive at least some of the first blood-derived sample material portion in the first detection zone, receive at least some of the second blood-derived sample material portion in the second detection zone, actuate the magnetic field system to cause the first and respective members to move blood within the first and second detection zones and form respective first and second mixtures, and the processor is configured to operate the first and second electrodes to determine respective values indicative of an amount of the first reagent present in the first and second mixtures.
108. A device, comprising:

a substrate that defines, at least in part, a microfluidic network comprising a first branch comprising a detection zone in communication with an inlet, and a second branch comprising a second detection zone in communication with an inlet,

dry cobalt salt disposed in the first branch,

a first magnetically susceptible member disposed in the first detection zone,

dry cobalt salt disposed in the second branch,

dry nickel salt disposed in the second branch,

a second magnetically susceptible member disposed in the second detection zone,

first electrodes in electrochemical communication with the first detection zone, and

second electrodes in electrochemical communication with the second detection zone,

a magnetic field system configured to magnetically manipulate the first'and second magnetically susceptible members,

a processor configured to actuate the magnetic field system,

wherein:

upon the receipt of a respective blood derived sample in the first and second detection zones, the processor actuates the magnetic field system to cause the first and respective members to move blood derived sample within the first and second detection zones and form respective first and second mixtures, the first mixture comprising solubilized cobalt, the second mixture comprising solubilized cobalt and solubilized nickel, and the processor is configured to operate the first and second electrodes to determine respective values indicative of an amount of cobalt present in the first and second mixtures.
109. The device of embodiment 108, wherein the values indicative of the amount of cobalt present in the first and second mixtures are indicative of the respective amounts of cobalt not complexed with albumin in the mixtures.
110. The device of embodiment 108 or 109, wherein the first and second branches are each in communication with a common junction and the junction is in communication with a common inlet.
111. A method, comprising:
   forming a first mixture comprising cobalt and a first sample, the first sample being derived from blood-derived sample material of a mammal,
   determining a first value indicative of an amount of cobalt not complexed with albumin present in the first mixture,
   forming a second mixture comprising cobalt and a second sample, the second sample being derived from blood-derived sample material of the mammal, a ratio of the mass of cobalt to the mass of second sample of the second mixture being at least 50% higher than a ratio of the mass of cobalt to the mass of first sample of the first mixture,
   determining a second value indicative of an amount of cobalt not complexed with albumin present in the second mixture, and
   based on at least the first and second values, determining a value indicative of an amount of albumin present in the first mixture.
112. The method of embodiment 111, wherein the ratio of the mass of cobalt to the mass of second sample of the second mixture is at least 75% higher than the ratio of the mass of cobalt to the mass of first sample of the first mixture.
113. The method of embodiment 111 or 112, wherein the ratio of the mass of cobalt to the mass of second sample of the second mixture is about twice the ratio of the mass of cobalt to the mass of first sample of the first mixture.
114. A device, comprising:

a substrate that defines, at least in part, a microfluidic network comprising an inlet in communication with a junction, a first detection zone in communication with the junction and a second detection zone in communication with the junction,

dry cobalt salt disposed within the microfluidic network,

first electrodes in electrochemical communication with the first detection zone, and

second electrodes in electrochemical communication with the second detection zone,

wherein:

the device is configured to receive a blood derived sample introduced to the inlet, partition the blood sample at the junction into first and second blood-derived sample material portions, form a first mixture comprising at least some of the first blood-derived sample material portion and at least some of the cobalt salt, and form a second mixture comprising at least some of the second blood-derived sample material portion, a ratio of the mass of cobalt to the mass of second sample of the second mixture being at least 50% higher than a ratio of the mass of cobalt to the mass of first sample of the first mixture.
115. The device of embodiment 114, further comprising a processor in communication with the first and second electrodes, wherein, in use, the processor is configured to operate the respective first and second electrodes to determine an amount of cobalt not complexed with albumin present in the first mixture and second mixtures.
116. A method, comprising:
   forming a first mixture comprising a first reagent and a first blood-derived sample material derived from blood of a mammal, the first reagent being capable of forming a complex with albumin present in the blood-derived sample material,
   determining a first value indicative of an amount of first reagent not complexed with albumin present in the first mixture,
   forming a second mixture comprising the first reagent and a second blood-derived sample material derived from blood of the mammal, a ratio of the mass of first reagent to the mass of second sample of the second mixture being at least 50% higher than a ratio of the mass of first reagent to the mass of first sample of the first mixture,
   determining a second value indicative of an amount of first reagent not complexed with albumin present in the second mixture, and
   based on at least the first and second values, determining a value indicative of an amount of albumin present in the first mixture.
117. A device, comprising:

a substrate that defines, at least in part, a microfluidic network comprising an inlet in communication with a junction, a first detection zone in communication with the junction and a second detection zone in communication with the junction, the first reagent being capable of forming a complex with albumin present in a blood-derived sample material,

a first reagent disposed within the microfluidic network,

first electrodes in electrochemical communication with the first detection zone, and

second electrodes in electrochemical communication with the second detection zone,

wherein:

the device is configured to receive a blood-derived sample material introduced to the inlet, partition the blood-derived sample material at the junction into first and second blood sample portions, form a first mixture comprising at least some of the first blood-derived sample material portion and at least some of the first reagent, and form a second mixture comprising at least some of the second blood-derived sample material portion and at least some of the first reagent, a ratio of the mass of first reagent to the mass of second sample of the second mixture being at least 50% higher than a ratio of the mass of first reagent to the mass of first sample of the first mixture.
118. An electrochemical device, comprising:

a substrate that defines, at least in part, a microfluidic network comprising an inlet in communication with a first detection zone,

a substrate that defines, at least in part, a microfluidic network comprising an inlet in communication with a second detection zone,

dry cobalt salt disposed within the microfluidic network comprising the first detection zone,

dry cobalt salt disposed within the microfluidic network comprising the second detection zone,

first electrodes in electrochemical communication with the first detection zone, and

second electrodes in electrochemical communication with the second detection zone,

wherein:

the device is configured to receive a blood derived sample introduced to the inlet in communication with the first detection zone, form a first mixture comprising at least some of the blood-derived sample material and at least some of the cobalt of the microfluidic network comprising the first detection zone, and form a second mixture comprising at least some of the blood-derived sample material, at least some of the cobalt of the microfluidic network comprising the second detection zone, a ratio of the mass of cobalt to the mass of second sample of the second mixture being at least 50% higher than a ratio of the mass of cobalt to the mass of first sample of the first mixture.
119. An electrochemical device, comprising:

a substrate that defines, at least in part, a microfluidic network comprising an inlet in communication with a first detection zone,

a substrate that defines, at least in part, a microfluidic network comprising an inlet in communication with a second detection zone,

a first reagent disposed within the microfluidic network comprising the first detection zone, the first reagent being capable of forming a complex with albumin,

first reagent disposed within the microfluidic network comprising the second detection zone,

first electrodes in electrochemical communication with the first detection zone, and

second electrodes in electrochemical communication with the second detection zone,

wherein:

the device is configured to receive a blood derived sample introduced to the inlet in communication with the first detection zone, form a first mixture comprising at least some of the blood-derived sample material and at least some of the first reagent of the microfluidic network comprising the first detection zone, and form a second mixture comprising at least some of the blood-derived sample material, at least some of the first reagent of the microfluidic network comprising the second detection zone, a ratio of the mass of first reagent to the mass of second sample of the second mixture being at least 50% higher than a ratio of the mass of first reagent to the mass of first sample of the first mixture.
120. A device, comprising:

means for forming a first mixture of a first cobalt reagent and blood-derived sample material of a mammal,

means for forming a first mixture of the first cobalt reagent, a second reagent, and blood-derived sample material of the mammal,

means for determining an amount of free cobalt in the first mixture,

means for determining an amount of free cobalt in the second mixture, and

means for determining the presence of ischemia in the mammal based on at least the amount of free cobalt in the first and second mixtures.
121. A method, including:

forming a first mixture including a first reagent and first sample material including an analyte, a first portion of the first reagent and the analyte forming a complex,

determining a second portion of the first reagent in the first mixture, the second portion of the first reagent not being complexed with the analyte,

forming a second mixture including first reagent, second sample material including the analyte, and a second reagent, the second reagent and the analyte interacting to prevent and/or reduce formation of the complex between the first reagent and the analyte, and

determining a third portion of the first reagent in the second mixture, the third portion of the first reagent not being complexed with the analyte.
122. The method of embodiment 121, further including determining the analyte in at least one of the first and second sample materials based on results of the detection of the second portion of the first reagent and the detection of the third portion of the first reagent.
123. The method of embodiment 122, wherein, the steps of determining are performed electrochemically within a microfluidic assay device including electrodes and the step of determining the analyte is performed using electronic circuitry within an assay device reader configured to operate the microfluidic device.
124. The method of embodiment 122, wherein determining the analyte comprises determining a difference between a result of the detection of the second portion of the first reagent and a result of detection of the third portion of the first reagent.
125. The method of embodiment 121, wherein forming the second mixture comprises combining at least a portion of the first mixture and the second reagent.
126. The method of embodiment 121, wherein determining the second portion of the first reagent and determining the third portion of the first reagent comprises electrochemically determining the second and third portions.
127. The method of embodiment 126, wherein forming the first mixture comprises contacting the first sample material with the first reagent with the first reagent being in dry form.
128. The method of embodiment 127, wherein the first reagent is in dry form along a surface of a microchannel of a microfluidic device when contacted by the first sample material.
129. The method of embodiment 121, wherein the first reagent comprises a metal or a salt thereof.
130. The method of embodiment 129, wherein the first reagent comprises a cobalt salt.
131. The method of embodiment 129, wherein the analyte comprises a protein.
132. The method of embodiment 129, wherein the analyte is albumin.
133. The method of embodiment 12, wherein the second reagent comprises a metal or salt thereof, the second reagent being different from the first reagent.
134. The method of embodiment 13, wherein the second reagent comprises a nickel salt.
135. The method of embodiment 13, wherein the first sample material and the second sample material have each been obtained from a mammal.
136. The method of embodiment 15, wherein the first and second sample materials each comprise a blood-derived sample material.
137. The method of embodiment 16, further including determining the analyte in at least one of the first and second sample materials based on results of the detection of the second portion of the first reagent and the detection of the third portion of the first reagent.
138. The method of embodiment 17, further including determining whether the mammal has suffered an ischemic event based on a result of the determination of the analyte.
139. The method of embodiment 18, wherein determining the second portion of the first reagent and determining the third portion of the first reagent comprises electrochemically determining the second and third portions.
140. The method of embodiment 19, wherein electrochemically determining comprises electrochemically determining the second and third portions using at least one common electrode.
141. A method, including:

forming a first mixture including a first reagent and first sample material including an analyte, a first portion of the first reagent and the analyte interacting to modify the first reagent,

determining a second portion of the first reagent in the first mixture using a technique insensitive to the first portion of the first reagent that has interacted with the analyte,

forming a second mixture including first reagent, second sample material including the analyte, and a second reagent, the second reagent and the analyte interacting to prevent and/or reduce interaction between the first reagent and the analyte, and

determining a third portion of the first reagent in the second mixture using a technique insensitive to the first portion of the first reagent that has interacted with the analyte.
142. The method of embodiment 21, further including determining the analyte in at least one of the first and second sample materials based on results of the detection of the second portion of the first reagent and the detection of the third portion of the first reagent.
143. The method of embodiment 22, wherein the sample material is the blood of a mammal and the method further comprises determining whether the mammal has suffered an ischemic event based on a result of the determination of the analyte.
144. The method of embodiment 21, wherein the modification of the first reagent by interaction with the analyte is the formation of complex between the first reagent and the analyte.
145. The method of embodiment 22, wherein, the steps of determining are performed electrochemically within a microfluidic assay device including electrodes and the step of determining the analyte is performed using electronic circuitry within an assay device reader configured to operate the microfluidic device.
146. The method of embodiment 21, wherein the first reagent comprises a metal or a salt thereof.
147. The method of embodiment 26, wherein the first reagent comprises a cobalt salt.
148. The method of embodiment 26, wherein the analyte comprises a protein.
149. The method of embodiment 26, wherein the second reagent comprises a metal or salt thereof, the second reagent being different from the first reagent.
150. The method of embodiment 29, wherein the second reagent comprises a nickel salt.
151. The method of embodiment 21, wherein determining the second portion of the first reagent and determining the third portion of the first reagent comprises electrochemically determining the second and third portions.
152. A device, including:

a first sample preparation zone configured to combine sample material and a first reagent including an analyte to form a first mixture, the first reagent and the analyte being capable of forming a complex,

a first detector configured to determine a first portion of the first reagent of the first mixture, the first portion of the first reagent not being complexed with the analyte,

a second sample preparation zone configured to combine sample material including analyte, first reagent, and a second reagent to form a second mixture, the second reagent and the analyte capable of interacting to prevent and/or reduce formation of the complex between the first reagent and the analyte,

a second detector configured to determine a second portion of the first reagent in the second mixture, the second portion of the first reagent not being complexed with the analyte, and

a processor configured to receive signals from the first and second detectors and determine the analyte in at least one of the first and second mixtures based on the signals.
153. The device of embodiment 32, wherein the first sample preparation zone comprises a cobalt salt and the first reagent is cobalt ion.
154. The device of embodiment 32, wherein the second sample preparation zone comprises a nickel salt and the second reagent is nickel ion.
155. The device of embodiment 32, wherein the processor is further configured to determine, when the sample material comprises a blood derived material from a mammal, whether the mammal has experienced an ischemic event based on the determination of the analyte.
156. A method for assaying an analyte in a sample material, including the steps of:

providing a reagent having a detectable characteristic and being capable of forming a chemical moiety with said analyte which moiety exhibits a change in said detectable characteristic of the reagent;

forming a first mixture including said reagent and sample material in which said analyte is to be assayed, under conditions conducive to formation of said chemical moiety from at least a portion of the reagent and said analyte;

detecting the presence in said first mixture of unchanged reagent;

forming a second mixture including said reagent, sample material in which said analyte is to be assayed, and a further reagent, under conditions conducive to formation of a chemical moiety from at least a portion of the reagent and said analyte, the further reagent and the analyte together preferentially forming a distinct chemical species to thereby inhibit formation of the chemical moiety;

detecting the presence in said second mixture of unchanged reagent; and

determining the presence of analyte in the sample therefrom.
157. The method of embodiment 36, additionally including the step of relating data from the detection steps to predetermined reference values associated with a disease condition.
158. A kit for clinical laboratory use including a plurality of containers each one of which contains a reagent having a detectable characteristic and being capable of interacting with a component of a sample of physiological fluid to form a chemical moiety therewith whereby the detectable characteristic of the reagent is modified; said kit further including a device configured to provide a sample deposit zone, at least one reagent treatment zone, means for introducing at least one reagent and sample to said treatment zone, wherein the device is adapted for positioning with respect to a detector whereby the detectable characteristics of the reagent(s) can be detected.
159. The method of any of the foregoing embodiments, wherein the blood-derived sample material comprises whole blood, plasma, serum, or combination thereof.
160. The method of any of the foregoing embodiments, wherein the blood-derived to sample material consists essentially of whole blood, plasma, serum, or combination thereof.
161. The device of any of the foregoing embodiments, wherein the blood-derived sample material comprises whole blood, plasma, serum, or combination thereof.
162. The device of any of the foregoing embodiments, wherein the blood-derived sample material consists essentially of whole blood, plasma, serum, or combination thereof.
163. Any of the foregoing embodiments in which the blood derived sample is whole blood.
164. Any of the foregoing embodiments in which the blood derived sample is serum.
165. Any of the foregoing embodiments in which the mammal is a human.
166. A reagent material comprising a salt of a first metal and at least one of a chloride compensating reagent, a suspension enhancing reagent, a plasticizing reagent, a dispersion reagent, a buffer reagent, antifoam reagent, or combination thereof, the metal having an affinity for the first metal.
167. A reagent material comprising a salt of a first metal, a salt of a second metal, and at least one of a chloride compensating reagent, a suspension enhancing reagent, a plasticizing reagent, a dispersion reagent, a buffer reagent, antifoam reagent, or combination thereof, the second metal having a higher affinity for albumin than the first metal.
168. The reagent material of embodiment 166 or embodiment 167, wherein the first metal is selected from the group consisting of V, As, Co, Cu, Sb, Cr, Mo, Mn, Ba, Zn, Ni, Hg, Cd, Fe, Pb, Au and Ag.
169. The reagent material of embodiment 168, wherein the first metal is Co.
170. The reagent material of embodiment 168 or 169, wherein the second metal is Ni.
171. The reagent material of any of embodiments 166 to 170 wherein the plasticizing reagent comprises a cellulose derivative.
172. The reagent material of any of embodiments 166 to 171 in which the reagent material is in a dry state.
173. Any of the foregoing devices comprising the reagent material of any of embodiments 166 to 172.
174. Any of the foregoing devices comprising the reagent material of any of embodiments 166 to 172, the reagent material not comprising the second metal and the reagent material of any of embodiments 166 to 172, the reagent material comprising the second metal.
175. Any of the foregoing devices comprising a first amount of the reagent material of any of embodiments 166 to 172, the first amount of reagent material having a first amount of the first metal, and a second amount of the reagent material of any of embodiments 166 to 172, the second amount of reagent material having a second, greater amount of the first metal.
176. The device of embodiment 174 or 175 in which one of the reagent materials is disposed in a first detection zone of the device and the other reagent material is disposed in a second detection zone of the device.
177. The device of embodiment 172, in which the reagent material is disposed as a screen-printed layer within the detection zone.
178. Any of the foregoing devices comprising a stir bar disposed in a detection zone of the device.
179. The device of embodiment 178, in which the detection zone has a volume of about 20 μl or less.
180. The device of embodiment 178 or 179 in which the detection zone further comprises a reagent material.
181. The device of embodiment 180 in which the stir bar is immobilized by a soluble reagent.
182. The device of any of embodiments 178 to 181 in which the stir bar is immobilized in the detection zone at a non-zero angle to a longitudinal axis of a channel along which sample material enters the detection zone.
183. The device of embodiment 181, wherein the soluble reagent is a reagent material of embodiment 166 or embodiment 167.
184. The device of embodiment 183, wherein the soluble reagent comprises a plasticizing reagent, for example, a cellulose derivative.
185. The device of embodiment 178 or 179 in which the detection zone further comprises any of the reagent materials of embodiment 166 or embodiment 167.
186. A method including introducing a sample into a detection zone of a device, the detection zone comprising at least one electrode, a surface opposed to the electrode, a sample material, a reagent material, and a stir bar, and subjecting the stir bar to a time-varying magnetic field, the magnetic field moving the stir bar and urging the stir bar against the opposed surface.
187. The method of embodiment 186, wherein the detection zone has a volume of about 10 μl or less.
188. The method of embodiment 186 or 187, performed using the device of any of the foregoing embodiments.
189. The method of embodiments 186 to 188 in which the reagent material is any of the reagent materials of embodiment 166 or embodiment 167.

Unless stated otherwise, the terms “albumin”, “normal albumin”, and “unmodified albumin” are synonymous and exclude ischemia modified albumin (IMA) with a reduced metal binding capacity.

Unless stated otherwise, a reference to a metal or in the context of a reagent material includes a reference to a salt of the metal.

Unless stated otherwise, a reference to V, As, Co, Cu, Sb, Cr, Mo, Mn, Ba, Zn, Ni, Hg, Cd, Fe, Pb, Au or Ag in the context of a reagent material includes a reference to a salt of V, As, Co, Cu, Sb, Cr, Mo, Mn, Ba, Zn, Ni, Hg, Cd, Fe, Pb, Au or Ag.

Unless stated otherwise, a reference to a metal in the context of a mixture including a sample material and a reagent material includes a reference to an ion of the metal or a solvate or other complex of the metal or an ion of the metal.

Unless stated otherwise, a reference to V, As, Co, Cu, Sb, Cr, Mo, Mn, Ba, Zn, Ni, Hg, Cd, Fe, Pb, Au or Ag in the context of a mixture including a sample material and a reagent material includes a reference to an ion of V, As, Co, Cu, Sb, Cr, Mo, Mn, Ba, Zn, Ni, Hg, Cd, Fe, Pb, Au or Ag or a solvate or other complex of V, As, Co, Cu, Sb, Cr, Mo,

Mn, Ba, Zn, Ni, Hg, Cd, Fe, Pb, Au or Ag or an ion of V, As, Co, Cu, Sb, Cr, Mo, Mn, Ba, Zn, Ni, Hg, Cd, Fe, Pb, Au or Ag.

Other features, objects, and advantages will be apparent from the description and drawings, and from the claims.

### BRIEF DESCRIPTION OF THE DRAWINGS

FIG. 1 is a flow chart of an assay method.

FIG. 2 is a graph illustrating an electrochemical signal as a function of cobalt concentration for each of three mixtures prepared in accord with the method of FIG. 1.

FIG. 3 is a flow chart of an assay method.

FIG. 4a is a top view of an assay device for performing an assay.

FIG. 4b is a perspective view of the device of FIG. 4a.

FIG. 4c is a partial cross section taken through a detection zone of the device of FIG. 4a.

FIG. 5a is a system including the assay device of FIG. 4 and an assay reader configured to operate the assay device.

FIG. 5b illustrates operation of a magnetic stir bar actuator of the assay reader of FIG. 5a.

FIG. 6a is a top view of an assay device for performing an assay.

FIG. 6b is an exploded perspective view of the assay device of FIG. 6a.

FIG. 7a is top view an assay reader.

FIG. 7b is a side view of the assay reader of FIG. 7a.

FIG. 7c is a top perspective view of the assay reader of FIG. 7a.

FIG. 7d is a bottom perspective view of the assay reader of FIG. 7a.

FIGS. 8a, 8b, 8c and 8d illustrate steps in an assembly sequence of the assay reader of FIG. 7a.

FIG. 9a is an exploded partial perspective side view of the assay reader of FIG. 7a and an assay device (e.g., the device of FIG. 4a or FIG. 6a).

FIG. 9b is an exploded partial end on view of the assay reader of FIG. 7a and an assay device.

FIG. 10a is an exploded partial perspective top end on view of the assay reader of FIG. 7a and an assay device.

FIG. 10b is a perspective view of the assay reader of FIG. 7a and an assay device.

FIG. 11a is an exploded partial perspective side view as in FIG. 9a but with the assay device fully inserted into the assay reader.

FIG. 11b is an exploded partial end on view as in FIG. 9b but with the assay device fully inserted into the assay reader.

FIG. 12 is an exploded partial perspective top end on view as in FIG. 10a but with the assay device fully inserted.

FIGS. 13a and 13b are exploded partial views of the device of FIG. 7a with the assay device partially inserted (FIG. 13a) and fully inserted (FIG. 13b) into the assay reader.

FIG. 14 illustrates a perspective view of the device of FIG. 7a illustrating an ejection button.

FIG. 15 is three exploded partial views illustrating a mechanism for ejecting an assay device from the assay reader of FIG. 7a.

FIG. 16 illustrates an assay device which can include microfluidic networks and components as described for the assay device of FIG. 4a or the assay device of FIG. 6a.

FIG. 17 is a top view of an assay device for performing an assay.

FIG. 18 is a top view of an assay device for performing an assay.

FIG. 19 is a graph illustrating amperometric cobalt reduction current vs. voltage plots for a 1 mM Co solution in whole blood and a 1 mM Co/5 mM Ni solution in whole blood from the same source.

FIG. 20 is a graph illustrating an amperometric cobalt reduction current vs. nickel concentration plot for a 1 mM Co solution in whole blood and an amperometric cobalt reduction current vs. copper concentration plot for a 1 mM Co solution in whole blood from the same source.

FIG. 21 is a graph illustrating an amperometric cobalt reduction current vs. cobalt concentration plot for cobalt concentrations ranging from 1 to 3 mM in whole blood from the same source as that of FIG. 20.

FIG. 22 is a graph illustrating an amperometric cobalt reduction current vs. nickel concentration plot for a 1.5 mM Co solution in serum and an amperometric cobalt reduction current vs. copper concentration plot for a 1.5 mM Co solution in serum from the serum having been prepared from whole blood from the same source as that of FIG. 20.

FIG. 23 is a graph illustrating an amperometric cobalt reduction current vs. cobalt concentration plot for cobalt concentrations ranging from 1.5 to 4 mM in serum and an amperometric cobalt reduction current vs. cobalt concentration plot for cobalt concentrations ranging from 0 to 4 mM in serum having a 20 mM Ni concentration, the serum having been prepared from whole blood from the same source as that of FIG. 20.

FIG. 24 is a graph that illustrates the effect of nickel concentration on the electrochemical signal from cobalt in whole blood.

FIG. 25 is a graph illustrating cobalt reduction current plotted against ACB value for each of multiple blood-derived samples.

FIG. 26 is a graph illustrating cobalt reduction current plotted against ACB value for each of multiple blood-derived samples measured in the presence of added nickel.

FIG. 27 is a graph illustrating cobalt reduction current plotted against ACB value.

FIG. 28 is a graph illustrating a ratio of measured currents plotted against ACB value.

### DETAILED DESCRIPTION

We describe assays for determining the presence (e.g., quantitatively or qualitatively) of one or more analytes in sample material. Exemplary assays include determining a result related to the amount of albumin in sample material (e.g., a blood derived sample material such as whole blood, plasma, serum, or combination thereof) obtained from a mammal (e.g., a human) suspected of having experienced (or experiencing) an ischemic event. Based on the result, the occurrence or non-occurrence of the ischemic event in the mammal is determined.

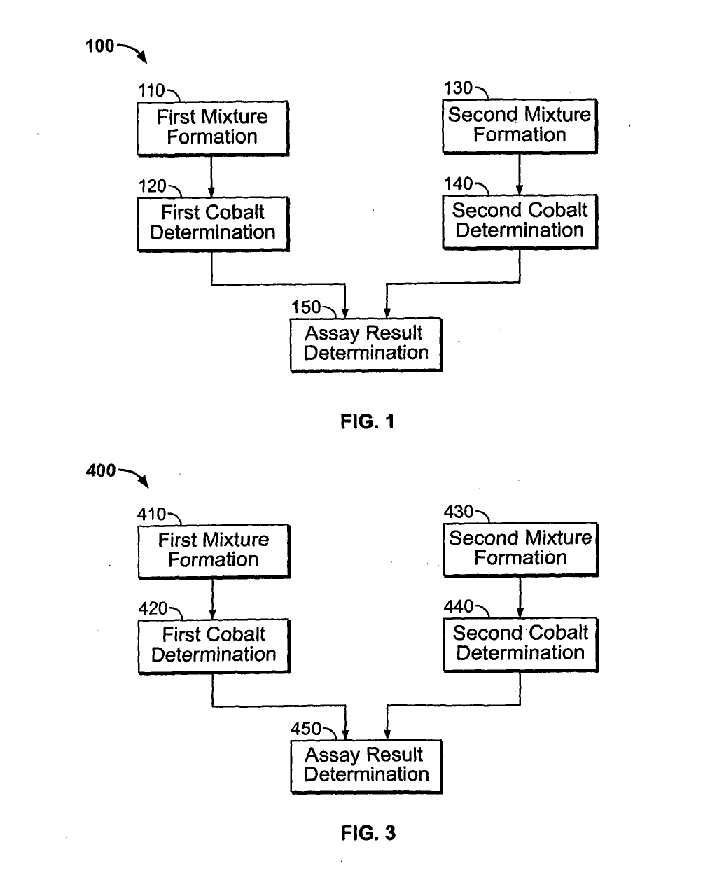

Referring to FIG. 1, an assay method 100 includes a first mixture formation step 110, a first cobalt electrochemical determination step 120, a second mixture formation step 130, a second cobalt determination step 140, and an assay result determination step 150. In first mixture formation step 110, a first mixture is formed. The first mixture includes cobalt and sample material from a human. In first cobalt determining step 120, cobalt free of cobalt-albumin complexes in the first mixture is determined. In second mixture formation step 130, a second mixture is formed. The second mixture includes cobalt, nickel, and sample material from the same human. In second electrochemical determining step 140, cobalt free of cobalt-albumin complexes in the second mixture is determined. In assay result determining step 150, an assay result is determined based on the results of first and second determining steps 120,140. The assay result is related to the amount of albumin in the blood of the human. Method 100 can further include determining the occurrence of an ischemic event in the human based on the assay result of step 150 (e.g., by comparing the assay result to a reference value). As discussed below, the result of second determining step 140 can operate as a control result that reduces the susceptibility of assay result of determination step 150 to matrix effects as compared to an assay result based on the result of first determining step 120 alone.

We now discuss method 100 in greater detail. Although various types of sample materials obtained from various sources (e.g., mammals) are suitable for use with methods described herein, we exemplify method 100 in non-limiting fashion with respect to the use of whole blood from a human as a sample material.

In first mixture formation step 110, a first mixture is formed by combining reagent material, which includes cobalt, and sample material, which includes whole blood from the human. In the first mixture, a first portion of the cobalt from the reagent material and albumin from blood of the human form cobalt-albumin complexes (e.g., a first portion of the cobalt is sequestered (e.g., bound) by albumin). A second portion of the cobalt from the reagent material remains free of albumin (e.g., unsequestered (e.g., unbound) by albumin). In the first mixture, the difference between the total amount of cobalt and the amount of cobalt free of albumin is related to (e.g., proportional to) the amount of albumin. For example, the difference increases as the amount of albumin increases because more albumin is available to sequester the cobalt. The difference decreases as the amount of albumin decreases because less albumin is available to sequester the cobalt.

The sample material (e.g., blood) can be obtained from the human as desired. In an exemplary embodiment, the sample material includes blood obtained by a “finger stick”, “venous draw”, or similar procedure. Blood can be used directly (e.g., as whole blood). A blood derived material (e.g., serum) can be used after processing blood to separate red blood cells. Typically, the sample material contains little or no chelators (e.g., EDTA) that compete with albumin to bind cobalt.

The total blood volume used in method 100 can be as desired. The total blood volume is the sum of the blood volumes used to perform first and second mixture formation steps 110,130. In general, the blood volumes used to perform first and second mixture formation steps 110,130 are about the same (e.g., the same).

Typically, the total volume of blood used in method 100 can be obtained with a small number (e.g., three or less, two or less, one) of “finger sticks”. For example, in some embodiments, the total volume of blood used in method 100 is obtained with a single “finger stick”. In some embodiments, the total volume of blood used in method 100 is about 100 μl or less (e.g., 75 μl or less, 50 μl or less, 30 μl or less). In some embodiments, the total volume of blood used in method 100 is about 5 μl or more (e.g., about 10 μl or more, about 15 μl or more). In an exemplary embodiment, the total volume of blood used in method 100 is about 20 μl.

The reagent material includes a cobalt reagent that provides cobalt ions when combined with the sample material. Typically, the cobalt reagent includes a solid (e.g., cobalt in the form of a cobalt salt (e.g., cobalt chloride, cobalt sulfate, cobalt acetate, cobalt nitrate, or combination thereof). In an exemplary embodiment, the reagent includes a dry cobalt salt (e.g., cobalt chloride). The sample material (e.g., whole blood) combines with and solubilizes the dry cobalt salt to form the first mixture. The reagent material may include a material such as polyvinyl alcohol (PVA). For example, the cobalt reagent (e.g., cobalt salt) can be combined with the PVA. As an alternative, or in combination, the cobalt reagent can include a liquid cobalt solution (e.g., an aqueous cobalt solution (e.g., a cobalt chloride solution)).

The concentration of cobalt in the first mixture is typically at least as high as (e.g., is higher than) the expected albumin concentration in the first mixture. The expected albumin concentration in the first mixture can be estimated from, for example, the expected albumin concentration in the sample material (e.g., blood-derived material) of the subject from which the sample material is obtained, the amount of the blood in the first mixture, and the volume of the first mixture.

The expected albumin concentration in the blood can be determined as desired. For example, the expected albumin concentration can be determined based on one or more prior determinations of the albumin concentration in sample material of the subject from which the sample material is obtained. As an alternative or in combination, the expected albumin concentration can be determined based on the average albumin concentration in the sample material (e.g., blood-derived material) from subjects similar to the subject from which the sample is obtained. The average albumin concentration in human blood typically depends on the individual's characteristics (e.g., age) and physiological state. In general, albumin concentrations of between about 300 μM and about 900 μM are observed with an average of about 750 μM.

In some embodiments, the cobalt concentration in the first mixture is at least about 500 μM (e.g., at least about 750 μM, at least about 1.0 mM, at least about 1.75 mM, at least about 3 mM). In some embodiments, the cobalt concentration in the first mixture is about 15 mM or less (e.g., about 10 mM or less, about 7.5 mM or less, about 5.0 mM or less). In an exemplary embodiment, the cobalt concentration in the first mixture is between about 0.75 mM and about 2 mM (e.g., about 1.0 mM). Unless specified otherwise, any concentration referred to herein is an “actual” concentration in which the volume of a mixture is determined by including the volume occupied by red blood cells. An “apparent” concentration is a concentration in which the volume of the mixture is determined by excluding the volume occupied by red blood cells. For example, a mixture prepared by combining 0.25 mg of CoCl₂ (formula weight 129.8) and 1 ml of whole blood having a 47% hematocrit has an actual cobalt concentration of about 1.9 mM (0.0025 g×129.8⁻¹ g×0.001⁻¹ l) and an apparent concentration of about 3.6 mM (0.0025 g×129.8⁻¹ g×0.00053⁻¹ l) because the red blood cells occupy a volume of about 0.47 ml of the mixture. Thus, when red blood cells are present, the “apparent” concentration is always larger than the actual concentration. For serum, the actual and apparent concentrations of solvated reagents are the same.

The reagent material used in first mixture formation step 110 may include a chloride compensating reagent. The chloride compensating reagent is discussed further below with respect to second mixture formation step 130.

The reagent material used in first mixture formation step 110 may include a suspension enhancing reagent configured to enhance the suspension of the cobalt reagent in the first mixture. An exemplary suspension enhancing reagent is a surfactant (e.g., a detergent) such as, for example, an octyl phenol ethoxylate, a polysorbate (e.g., Tween 80), Surfynol, Pluronic, a dodecylether,), a sulfosuccinamate, an organosilicone (e.g., Silwet), or a secondary alcohol athoxylate (e.g., Tergitol 15-S-9, Dow Chemical Company), or combination thereof. The surfactant can be nonionic, anionic, cationic, or amphoteric. Other suspension agents include poly alcohols (e.g., sugar alcohols such as sorbitol, zylitol, and erthrythritol) and sugars (e.g., trehalose and sucrose).

The reagent material used in first mixture formation step 110 may include a plasticizing reagent configured to reduce loss of the reagent material (e.g., prevent fragmentation and dusting) during storage and/or facilitate manufacture of a device for performing method 100 (e.g., as a binder or film former). An exemplary plasticizing reagent is a cellulose derivative (e.g., a hydroxyethylcellulose such as Natrosol G and Natrosol L, Agualon, carboxymethyl cellulose, hydroxypropyl cellulose, or combination thereof). Exemplary cellulose derivatives have a molecular weight between about 9.0×10⁴ and about 7.2×10⁵. Exemplary cellulose derivatives have a degree of polymerisation of between about 300 and about 1,100. The degree of polymerization, is the number of repeat units in an average polymer chain at time t in a polymerization reaction. In exemplary embodiments, the reagent material is between about 0.2% and 10% (e.g., about 1%) by weight plasticizing reagent when in the liquid state. Other plasticizing reagents include polyvinyl alcohol, polyvinyl pyrrolidone, and polyvinyl pyrrolidone/vinyl acetate.

The reagent material used in first mixture formation step 110 may include a dispersion reagent. An exemplary dispersion reagent includes a silica powder (e.g., Cabosil TS610). In general, organic or inorganic pigments which do not dissolve in water and have a size approximately anywhere between 0.1 and 10 microns could be used as dispersion reagents.

A reagent may perform more than one function. For example, a suspension enhancing reagent may also act as a plasticizing reagent.

The reagent material used in first mixture formation step 110 may include a buffer reagent configured to buffer a pH of the reagent as compared to the sample material (e.g., blood, serum, or other blood-derived material). For example, the buffer reagent may be configured to buffer the pH of the reagent-sample material mixture to have a pH of about the same as the sample material. An exemplary buffer has a pH of between about 7 and about 7.8 (e.g., between about 7.2 and about 7.6) when combined with a blood-derived sample. 3-(N-Morpholino)-propanesulfonic acid (MOPS) is an exemplary buffer material. Other buffer materials include phosphate, N-(2-hydroxyethyl)-piperazine-N′-2-ethanesulfonic acid (HEPES), imidazole, and citrate buffers.

The reagent material used in first mixture formation step 110 may include an antifoam reagent configured to minimize formation of the reagent material during production and/or during production of a device including the reagent material. An exemplary antifoam reagent is a silicone emulsion (e.g., Antifoam FDP, Basildon Chemicals).

Continuing with method 100, first electrochemical determination step 120 includes obtaining an electrochemical signal related to the amount (e.g., concentration) of cobalt that is free of albumin in the first mixture. Because the amount of free cobalt is generally reduced by the presence of albumin in the first mixture (and generally increases as the amount of IMA increases in the first mixture), the magnitude of the electrochemical signal is generally inversely proportional to the amount of albumin in the first mixture (and generally proportional to the amount of IMA in the first mixture). The amount of free cobalt decreases as the amount of albumin increases because more albumin is available to sequester the cobalt.

In an exemplary embodiment, first electrochemical determination step 120 is performed amperometrically. Cobalt free of albumin can be determined amperometrically by, for example, scanning the potential of a working electrode while measuring a current resulting from a reduction or oxidation of cobalt ion in the first mixture. Because cobalt in cobalt-albumin complexes essentially does not participate in the redox reaction, the current is indicative of (e.g., proportional to) the amount of free cobalt. The current can be measured, for example, as the maximum absolute value of the current within a potential range or as the integrated current within a potential range. The potential range typically covers potentials at which cobalt redox reactions occur (e.g., potentials of between about 0.6 V and about 0.8 V). The electrochemical signal is indicative of the current. In an exemplary embodiment, the voltage is scanned over a range of about +1 Volt to −0.5 V and the electrochemical signal is determined based on current measured between about 0.6 V and about 0.8 V. An exemplary voltage scanning rate is between about 0.4 V/s and about 1.0 V/s (e.g., about 0.7 V/s).

Typically, the first mixture is allowed to contact electrodes used to obtain the electrochemical signal for an electrode incubation time prior to making the electrochemical determination. During the electrode incubation time, the working electrode can be maintained at a non-zero voltage. In general, the electrode incubation time is about 240 seconds or less (e.g., about 120 seconds or less, about 60 seconds or less). In an exemplary embodiment, the incubation time is about 40 seconds and the working electrode is maintained at +1 V relative to a Ag—AgCl (silver-silver chloride) reference electrode.

An exemplary electrochemical detection can be performed as follows. Electrodes and reagent material are contacted with sample material to form a mixture. Sample material is applied to electrode and the working electrode is held at 0 V for 120 seconds. The working electrode is polarized at +1 V vs Ag/AgCl reference and held for 40 seconds. Working electrode potential is swept from +1V to −0.5V at 700 mV/s and the resultant current due to reduction of Co III to Co II is measured. The electrochemical signal due to free cobalt is determined from the peak height of the current v potential curve at about +0.7 V (peak height is determined by calculating the difference between the base of the peak (determined as the most positive current value between +0.4 and −0.2 V) and the peak (determined as the minimum current value between +1 and +0.4 V).

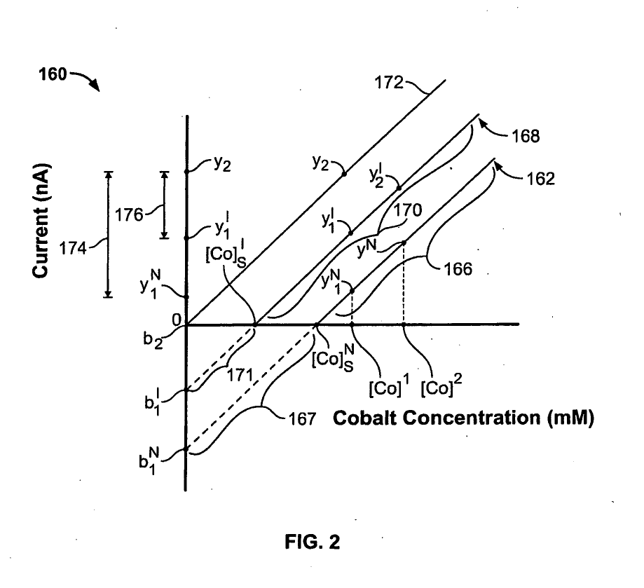

Referring to FIG. 2, a graph 160 illustrates a current response curve 162, which is the absolute value of the reduction current that would be obtained as a function of cobalt concentration in a first mixture prepared using blood of a non-ischemic subject according to first mixture formation step 110. A current response curve 168 is the absolute value of the reduction current that would be obtained as a function of cobalt concentration in a first mixture prepared using blood of an ischemic subject according to first mixture formation step 110. The values plotted along the y-axis are the absolute value of the difference between the negative peak of the reduction current between 0.6 V and 0.8 V and an inflection point of the current vs. voltage scan, which occurs at a voltage between about 0.4 and 0.6 V.

For curves 162,168, a respective saturation cobalt concentration [Co]ₛ^{N}, [Co]ₛ¹ of blood of a non-ischemic (N) or ischemic (I) subject corresponds to the cobalt concentration which equals the cobalt sequestering capacity of albumin in the mixture. The saturation cobalt concentration is proportional to the albumin concentration of that mixture. For cobalt concentrations lower the saturation cobalt concentration, essentially all of the cobalt is complexed by albumin and the current is essentially zero. For cobalt concentrations higher than the saturation cobalt concentration, essentially all of additional cobalt is present as cobalt free of albumin complexes and the current increases linearly with cobalt concentration. For example, a positive portion 166 of curve 162 at concentrations above [Co]ₛ^{N} is defined by the equation (Eq. 1) y=m[Co]+b₁^{N}, where y is the current, m is the slope, [Co] is the actual cobalt concentration, and b₁^{N} is the intercept. A negative portion 167 of curve 162 is the extrapolation of positive portion 166. As seen in FIG. 2, intercept b₁^{N} is negative because curve 162 is shifted to the right by an amount related to saturation cobalt concentration [Co]ₛ^{N}. A portion positive 170 of curve 168 at concentrations above [Co]ₛ¹ is defined by the equation (Eq. 2) y=m[Co]+b₁^{I}, where b₁^{I} is the intercept. A negative portion 171 of curve 168 is the extrapolation of positive portion 170. The amount of albumin in (and the saturation cobalt concentration of) the first mixture decreases as the amount of IMA increases. Accordingly, saturation cobalt concentration [Co]ₛ^{N} of the non-ischemic mixture is shifted to a higher cobalt concentration than saturation cobalt concentration [Co]ₛ^{I} of the ischemic mixture.

Typically, the electrochemical determination is more sensitive to cobalt that is free of albumin complexes than to cobalt in a cobalt-albumin complex. Thus, the contribution of free cobalt to the electrochemical signal is higher than the contribution of cobalt in cobalt-albumin complexes to the electrochemical signal. In some embodiments, the contribution of free cobalt to the electrochemical signal (e.g., current) is at least about 5 times higher (e.g., at least about 7.5 times higher, at least about 10 times higher, at least about 20 times higher) than the contribution of cobalt present in cobalt-albumin complexes.

Continuing with method 100, in second mixture formation step 130, a second mixture is formed by combining reagent material, which includes cobalt and nickel, and sample material, which, in this exemplification of method 100, includes blood from the human that supplied the blood used in first mixture formation step 120. In the second mixture, as discussed with respect to first mixture formation step 120, a first portion of the cobalt from the reagent material and albumin from blood of the human form cobalt-albumin complexes. A second portion of the cobalt from the reagent material remains free of albumin. The cobalt concentration in the second mixture is typically about the same as (e.g., the same as) the cobalt concentration in the first mixture. However, because the affinity of albumin for nickel is higher than the affinity of albumin for cobalt, albumin preferentially forms nickel-albumin complexes as compared to cobalt-albumin complexes. Thus, the presence of nickel increases the amount of cobalt free of albumin in the second mixture as compared to the first mixture.

The sample material used in second mixture formation step 130 may have the same properties (e.g., volume, type, and/or source) as that used in first mixture formation step 120. Typically, the sample material used in mixture formation steps 110,130 are obtained from the same source (e.g., from the same subject). Typically, the sample material used in mixture formation steps 110,130 are obtained contemporaneously. In an exemplary embodiment, the sample material used in first and second mixture formation steps 120,130 is a blood derived material (e.g., whole blood, plasma, serum, or combination thereof) obtained contemporaneously. For example, the blood used in first and second mixture formation steps 120,130 can be from blood produced from only a few (e.g., three or less, two or less, only one) “finger sticks”.

The reagent material of second mixture formation step 130 includes a cobalt reagent that provides cobalt ions when combined with the sample material and a nickel reagent that provides nickel ions when combined with the sample material. The cobalt reagent is generally the same as the reagent used in first mixture formation step 110 (e.g., a cobalt salt such as cobalt chloride, cobalt sulfate, cobalt acetate, cobalt nitrate, or combination thereof). The cobalt concentration in the second mixture is typically the same as that obtained in first mixture formation step 110.

Typically, the nickel reagent includes a solid (e.g., nickel in the form of a nickel salt (e.g., nickel chloride (NiCl₂))). In an exemplary embodiment, the reagent includes dry cobalt salt and dry nickel salt. The sample material (e.g., whole blood) combines with and solubilizes the dry cobalt and nickel salts to form the second mixture. The reagent material of the second mixture can be combined with PVA as discussed with respect to first mixture formation step 110. As an alternative, or in combination, the nickel reagent can include a liquid nickel solution (e.g., an aqueous nickel solution (e.g., a nickel chloride solution)).

In general, the nickel concentration in the second mixture is high enough that essentially no cobalt-albumin complexes are present (e.g., most or substantially all of the cobalt is prevented from forming cobalt-albumin complexes and/or is displaced from cobalt-albumin complexes). By essentially no cobalt-albumin complexes, it is meant that the cobalt-albumin complex concentration in the second mixture is smaller than (e.g., less than about 20% of, less than about 10% of, less than about 5% of, or less) the albumin concentration (not including albumin in the form of IMA) in the second mixture.

Typically, the total nickel concentration in the second mixture is higher than the cobalt concentration in the second mixture. For example, a ratio of the total nickel to cobalt concentrations is typically at least about 1.5 (e.g., at least about 3, at least about 5, at least about 10). The ratio of the total nickel to cobalt concentrations is typically about 25 or less (e.g., about 20 or less, about 15 or less, about 10 or less). In exemplary embodiments, the ratio of the total nickel to cobalt concentrations is between about 3 and about 8 (e.g., the ratio is about 5.5).

In some embodiments, the total nickel concentration in the first mixture is at least about 3 mM (e.g., at least about 5 mM, at least about 7.5 mM, at least about 10 mM, at least about 15 mM). In some embodiments, the total nickel concentration in the first mixture is about 30 mM or less (e.g., about 25 mM or less, about 20 mM or less, about 15 mM or less). In exemplary embodiments, the cobalt concentration in the second mixture is between about 0.75 mM and about 2 mM (e.g., about 1.0 mM) and the total nickel concentration in the second mixture is between about 3 mM and about 20 mM (e.g., is about 7.5 mM).

As discussed above, the reagent material used in first mixture formation step 110 may include a chloride compensating reagent. In general, the chloride compensating reagent provides more uniform electrochemical determinations between first and second determination steps 120,140.

Typically, the chloride compensating reagent balances the total chloride ion concentration in the first mixture with the chloride ion concentration in the second mixture, which may be higher if the nickel reagent includes nickel chloride. In some embodiments, therefore, the chloride compensating reagent in the first mixture contributes a total chloride concentration at least about the same as (e.g., the same as) the chloride concentration due to chloride ion of the nickel chloride. For example, if the second mixture includes 7.5 mM NiCl₂, the chloride compensating reagent of the first mixture is selected to contribute about 15 mM chloride ion to the first mixture.

In some embodiments, chloride compensating reagent is used in both first and second mixture formation steps 110,130. In these embodiments, the contribution by the chloride compensating reagent to both the first and second mixtures typically increases the chloride ion concentration to a level that provides more stable electrochemical determinations for first and second determination steps 120,140. For example, the amount of chloride ion contributed by the chloride compensating reagent can be selected to stabilize a Ag—AgCl reference electrode.

In some embodiments, each of the first and second mixtures includes sufficient chloride compensating reagent to increase the total chloride ion concentration from all reagent sources (not including chloride ion originating from the sample material) in the first and second mixtures to at least about 50 mM (e.g., at least about 75 mM, at least about 100 mM, at least about 125 mM). In some embodiments, each of the first and second mixtures includes sufficient chloride compensating reagent to increase the total chloride ion concentration from all reagent sources (not including chloride ion originating from the sample material) in the first and second mixtures by about 200 mM or less (e.g., about 175 mM or less, about 150 mM or less, about 125 mM or less). In an exemplary embodiment, each of the first and second mixtures includes sufficient chloride compensating reagent to increase the total chloride ion concentration from all reagent sources (not including chloride ion originating from the sample material) in the first and second mixtures by about 100 mM.

The chloride compensating reagent is typically selected so that the counter ion to chloride does not compete with cobalt to form albumin complexes. An exemplary chloride compensating reagent is alkali metal chloride salt (e.g., potassium chloride (KCl)).

The reagent material used in second mixture formation step 130 may include any combination of the reagent materials described with respect to first mixture formation step 110 (e.g., a chloride compensating reagent, a suspension enhancing reagent, a plasticizing reagent, a dispersion reagent, a buffer reagent, antifoam reagent, or combination thereof).

Continuing with method 100, second electrochemical determination step 140 includes obtaining an electrochemical signal related to the amount (e.g., concentration) of cobalt that is free of albumin in the second mixture. As discussed with respect to second mixture formation step 130, the second mixture contains essentially no cobalt-albumin complexes because of the presence of nickel. Accordingly, the amount of free cobalt in the second mixture and the resulting electrochemical signal are generally proportional to the total amount of cobalt in the second mixture. For example, an electrochemical signal y₂ (FIG. 2) is proportional to the amount (e.g., concentration) of cobalt that is free of albumin in the second mixture.

The electrochemical determination of step 140 is performed using the same electrochemical technique used to perform determination step 120. In an exemplary embodiment, determination step 140 is performed amperometrically as discussed for step 120. For example, free cobalt can be determined in the presence of nickel in the second mixture by measuring a current resulting from a cobalt redox reaction within a potential range of between about 0.6 V and about 0.8 V. The electrochemical signal is indicative of the measured current.

Returning to FIG. 2, graph 160 illustrates a current response curve 172, which is the absolute value of the reduction current that would be obtained as a function of cobalt concentration in a second mixture prepared according to second mixture formation step 130. The current is the absolute value of the peak reduction current between 0.6 V and 0.8 V. Response curve 172 is defined by the equation (Eq. 3) y=m[Co]+b₂, where y is the current, m is the slope, [Co] is the apparent cobalt concentration, and b₂ is the intercept. As seen in FIG. 2, intercept b₂ is essentially zero because, due to the presence of nickel, curve 172 is not shifted to the right by an amount related to the saturation cobalt concentration. Essentially all of the cobalt is present as cobalt free of albumin complexes and the measured current along curve 172 increases linearly with cobalt concentration. Thus, curve 172 is representative of such curves obtained from both ischemic and non-ischemic sample material. Typically, the slope m is about the same for curves 162,168,170.

Continuing with method 100, assay result determination step 150 includes determining an assay result based at least in part on the electrochemical signals of first and second determining steps 120,140. The results obtained in the first and second determining steps are processed in a suitable way, as will be described in the following. As discussed with respect to first determination step 120, the absolute value of the electrochemical signal determined in the absence of nickel is generally inversely proportional to the amount of albumin in the first mixture (and generally proportional to the amount of IMA in the first mixture). As discussed with respect second determination step 140, the electrochemical signal determined in the presence of nickel is generally proportional to the total amount of cobalt in the second mixture (and is essentially independent of the amounts of albumin or IMA).

In an exemplary embodiment, assay result determination step 150 includes determining a difference value indicative of the magnitude of the difference between the results of first and second determination steps 120,140 (e.g., by subtracting the result of first determination step 120 from the result of second determination step 140). As seen in FIG. 2, a difference value 174 corresponds to a difference between electrochemical signal y₂ obtained according to determination step 140 in a second mixture having a cobalt concentration [Co]¹ and an electrochemical signal y₁^{N} obtained according to determination step 120 from non-ischemic sample material in a first mixture having a cobalt concentration [Co]¹. A difference value 176 corresponds to a difference between electrochemical signal y₂ and an electrochemical signal y₁^{I} obtained according to determination step 120 from ischemic sample material in a first mixture having a cobalt concentration [Co]¹. In general, the magnitude of the difference value is indicative of (e.g., proportional to) the amount of albumin in the sample material used to form the first and second mixtures. For example, difference value 174 from the non-ischemic sample material is greater than difference value 176 from the ischemic sample material contains a reduced amount of albumin (and a greater amount of IMA).

The difference value can be determined as desired. Typically, the difference value is determined electronically (e.g., using analog or digital circuitry or a combination thereof). For example, in some embodiments, the difference value is determined using an analog subtraction technique. The electrical signals (e.g., currents or corresponding voltages) determined in first and second determination steps 120,140 are provided to an analog subtraction circuit. Such a circuit can be formed using, for example, an op amp and a resistor. The output of the circuit is indicative of the difference value. As another example, the difference value can be determined digitally. Each of the electrical signals determined in the first and second determining steps 120,140 is converted to a digital value and provided to a digital subtraction circuit. The output of the circuit is indicative of the difference value. As yet another example, the difference value may be determined using a processor, which can include a combination of analog and digital components (e.g., as in a microchip).

The assay result of determining step 150 can be used to determine whether the human from which the sample material was obtained has experienced or is experiencing an ischemic event. For example, the presence of an ischemic event can be determined by comparing a difference value determined using sample material (e.g., blood such as whole blood) with a reference value. A reference value can be determined using, for example, sample material obtained from one or more subjects known to have experienced or be experiencing an ischemic event, sample material obtained from one or more healthy subjects (e.g., subjects not suspected of having experienced or experiencing an ischemic event), sample material obtained from the same subject, or a combination thereof.

An exemplary reference value is a difference value determined using the same type of sample material obtained from one or more subjects known to have experienced or be experiencing an ischemic event. The presence of an ischemic event in a subject is diagnosed if, for example, a difference value determined by applying method 100 to sample material obtained from the subject falls below the corresponding reference difference value.

Another exemplary reference value is a difference value determined using the sample type of sample material obtained from one or more healthy subjects (e.g., non-ischemic subjects). The absence of an ischemic event in a subject is diagnosed if, for example, a difference value determined by applying method 100 to sample material obtained from the subject exceeds the corresponding reference difference value.

Another exemplary reference value is a difference value determined using sample material obtained on one or more previous occasions from the same subject. For example, method 100 can be applied to sample material obtained from a subject on each of one or more occasions. An assay result (e.g., difference value) is determined for the sample obtained on each occasion. The different occasions can be separated in time (e.g., by hours, days, weeks, or months). A reference value (e.g., baseline assay result) is determined from one or more of the previous assay results. For example, the reference value may be the average value of the difference value for multiple previous results. Subsequent assay results can be compared to the reference value of that subject. The absence of an ischemic event is diagnosed if, for example, the absolute value of a difference value determined by applying method 100 to sample material obtained from the subject falls below that subject's reference value.

As discussed above, the result of second determining step 140 can operate as a control result that makes the assay result of determination step 150 (e.g., a difference value) less susceptible to errors caused by matrix effects than if the assay result were based on the result of first determining step 120 alone. This advantage results from the way in which matrix effects can bias the results of determination steps 120,140 as compared to the true results of these steps (e.g., results that would be obtained in the absence of matrix effects). Matrix effects in the first and second mixtures typically bias the results of determinations 120,140 in a similar fashion (e.g., in the same direction (e.g., higher or lower) and/or to a similar magnitude). Assay result determination step 150 reduces the magnitude of the matrix effects by combining the results of determination steps 120,140. The assay result of determination step 150 is less susceptible to errors caused by matrix effects than if the assay result were based on the result of first determining step 120 alone. Accordingly, the presence of ischemia can be determined more reliably than if the assay result was based on the result of first determination step alone.

While determination step 150 has been described as including the determination of a difference value, other embodiments can be performed. For example, assay result determination step 150 can include determining a ratio value indicative of a ratio of the results of first and second determination steps 120,140. For example, a ratio value can be formed by dividing the electrochemical signal of first determination step 120 by the electrochemical signal of second determination step 140 (e.g., as y₁^{I}/y₂). Such a ratio value is indicative of (e.g., inversely proportional to) the amount of IMA in the sample material used to form the first and second mixtures. For example, the presence of an ischemic event can be determined by comparing a ratio value determined using sample material (e.g., blood such as whole blood) with a reference value. A reference value can be determined using, for example, sample material obtained from one or more subjects known to have experienced or be experiencing an ischemic event, sample material obtained from one or more healthy subjects (e.g., subjects not suspected of having experienced or experiencing an ischemic event), sample material obtained from the same subject, or a combination thereof.

As another example, assay result determination step 150 can include determining a difference ratio value. For example, a ratio difference value can be formed by dividing a difference value by the electrochemical signals of one or both of the first and second determination steps 120,140 (e.g., as [y₁^{I}−y₂]/y₂ or [y₁^{I}−y₂]/(y₁^{I}×y₂)). Such a difference ratio value is indicative of (e.g., inversely proportional to) the amount of IMA in the sample material used to form the first and second mixtures. For example, the presence of an ischemic event can be determined by comparing a difference ratio value determined using sample material (e.g., blood such as whole blood) with a reference value. A reference value can be determined using, for example, sample material obtained from one or more subjects known to have experienced or be experiencing an ischemic event, sample material obtained from one or more healthy subjects (e.g., subjects not suspected of having experienced or experiencing an ischemic event), sample material obtained from the same subject, or a combination thereof.

Assay results determined as difference values, ratio values, or difference ratio values all share the advantage of being less susceptible to errors caused by matrix effects than if the assay result was based on the result of first determining step 120 alone. Accordingly, the presence of ischemia can be determined more reliably than if the assay result was based on the result of first determination step alone. Assay result determination step 150 can alternatively (or in combination) include forming other combinations of the electrochemical signals of determination steps 120,140 besides difference, ratio, and difference ratios. In general, such combinations share the advantages discussed above and can also be used to determine the presence of ischemia.

While method 100 has been exemplified using whole blood, other sample materials can be used. For example, the sample material can alternatively be or additionally include a blood derived material (e.g., plasma, serum, or combination thereof). Typically, when a material that lacks red blood cells (e.g., plasma or serum) is substituted for (or combined with) whole blood, greater amounts of reagents are used to compensate for the greater available volume resulting from the lack of red blood cells.

In some embodiments, method 100 is performed using serum and/or plasma. In such embodiments, the cobalt concentration in the first and second mixtures is typically at least about 1 mM (e.g., at least about 1.25 mM, at least about 1.75 mM, at least about 3 mM, at least about 4 mM). In some embodiments, the total cobalt concentration in the first mixture is about 25 mM or less (e.g., about 20 mM or less, about 15 mM or less, about 10 mM or less). In an exemplary embodiment, the cobalt concentration in the first mixture is between about 1 mM and about 3.5 mM (e.g., about 1.5 mM). Because the serum and plasma lack red blood cells, the apparent and actual concentrations are the same.

Using serum and/or plasma, the ratio of the amount of nickel to cobalt reagent can be the same as described for second mixture formation step 130. The nickel concentration in the first mixture is typically at least about 4 mM (e.g., at least about 6 mM, at least about 9 mM, at least about 12 mM, at least about 17 mM). In some embodiments, the total nickel concentration in the first mixture is about 40 mM or less (e.g., about 30 mM or less, about 25 mM or less, about 20 mM or less). In exemplary embodiments, the cobalt concentration in the second mixture is between about 1 mM and about 3.5 mM (e.g., about 1.5 mM) and the nickel concentration in the second mixture is between about 4 mM and about 25 mM (e.g., is about 10 mM).

Other than the changes with respect to cobalt and nickel concentrations, method 100 can be performed the same with plasma and/or serum as with whole blood. For example, the reagent material used in the first and second mixture formation steps with serum and/or plasma may include chloride compensating reagent, suspension enhancing reagent and/or a plasticizing reagent as described with respect to first and second mixture formation steps 110,130. The volumes of sample material may be the same as for the first and second mixture formation steps 110,130. Determination of electrochemical signals and an assay result may be performed the same as for steps 120,140,150. An ischemic event may be determined as described using whole blood, with reference values obtained using plasma and/or serum.

While assay methods that include forming a second mixture including cobalt and a second reagent (e.g., nickel) that results in a reduced amount of cobalt-albumin complexes have been described, other embodiments can be performed.

Referring to FIG. 3, an assay method 400 includes a first mixture formation step 410, a first cobalt determination step 420, a second mixture formation step 430, a second cobalt determination step 440, and an assay result determination step 450. Although various types of sample materials are suitable for use with methods described herein, we first exemplify method 100 with respect to the use of whole blood as a sample material.

In first mixture formation step 410, a first mixture including cobalt and blood from a human is formed. In first cobalt electrochemical determining step 420, cobalt free of cobalt-albumin complexes in the first mixture is determined. In second mixture formation step 430, a second mixture including an amount of cobalt different from the amount of cobalt in the first mixture and blood from the human is formed. In second cobalt electrochemical determining step 440, cobalt free of cobalt-albumin complexes in the second mixture is determined. In assay result determining step 450, an assay result is determined based on results of first and second determining steps 420,440. Typically, the assay result is related to the amount of albumin in the blood of the human. Method 400 can further include determining the occurrence of an ischemic event in the human based on the result of assay determining step 450 (e.g., by comparing the assay result to a standard value).

In first mixture formation step 410, a first mixture is formed by combining reagent material, which includes cobalt, and sample material, which includes blood from the human. First mixture formation step 410 can be performed as described for first mixture formation step 110 of method 100. For example, the reagent material and sample material may be of the same types, amounts, and volumes as described for step 110 of method 100. In an exemplary embodiment, the reagent material of step 410 is a dry cobalt salt (e.g., cobalt chloride) and the sample material is whole blood (e.g., from one or more “finger sticks”). In an exemplary embodiment, the cobalt concentration in the first mixture is between about 0.75 mM and about 2 mM (e.g., about 1.0 mM).

The reagent material used in second mixture formation step 410 may include any combination of the reagent materials described with respect to mixture formation steps 110, 130 (e.g., a chloride compensating reagent, a suspension enhancing reagent, a plasticizing reagent, a dispersion reagent, a buffer reagent, antifoam reagent, or combination thereof).

First cobalt electrochemical determination step 420 includes electrochemically determining cobalt that is free of albumin complexes in the first mixture. The result of determination step 420 is an electrochemical signal related to the amount (e.g., concentration) of cobalt that is free of albumin in the first mixture. First cobalt determination step 420 can be performed as described for first determination step 120 of method 100. For example, cobalt determination step 420 can include amperometrically determining free cobalt using the same amperometric conditions as described for first determination step 120. In exemplary embodiment, first determination step 420 includes amperometrically determining free cobalt by measuring a current resulting from a redox reaction of cobalt ion that occurs within a potential range of between about 0.6 V and about 0.8 V.

Continuing with method 400, in second mixture formation step 430, a second mixture is formed by combining reagent material, which includes a greater amount of cobalt than that used in first mixture formation step 410, and sample material, which includes blood from the human that supplied the blood used in first mixture formation step 420. For example, as seen in FIG. 2, second mixture formation step 430 includes forming a second mixture having a cobalt concentration [Co]², which is greater than cobalt concentration [Co]¹ of the first mixture formed in step 410.

In the second mixture, as discussed with respect to first mixture formation step 120, a first portion of the cobalt from the reagent material and albumin from blood of the human form cobalt-albumin complexes. A second portion of the cobalt from the reagent material remains free of albumin. However, because the cobalt concentration [Co]² in the second mixture is higher than the cobalt concentration [Co]¹ in the first mixture, more free cobalt is present in the second mixture than in the first mixture.

In some embodiments, a ratio of the cobalt concentration in the second mixture (step 430) to the total amount of cobalt in the first mixture (step 410) is at least about 1.25 (e.g., at least about 1.5, at least about 2). In some embodiments, the ratio of the cobalt concentration in the second mixture (step 430) to the total amount of cobalt in the first mixture (step 410) is about 5 or less (e.g., about 3 or less about 2.5 or less). In an exemplary embodiment, the ratio of the cobalt concentration in the second mixture (step 430) to the total amount of cobalt in the first mixture (step 410) is about 2.

In exemplary embodiments, the cobalt concentration in the second mixture is about 3.5 mM or less. For example, the cobalt concentration in the first mixture may be about 1.0 mM and the cobalt concentration in the second mixture may be about 2 mM.

The reagent material used in second mixture formation step 430 may be identical with the material used in first mixture formation step 410 except that the amount cobalt reagent is selected to provide a higher cobalt concentration in the second mixture. The reagent material used in second mixture formation step 430 may include any combination of the reagent materials described with respect to mixture formation steps 110, 130 (e.g., a chloride compensating reagent, a suspension enhancing reagent, a plasticizing reagent, a dispersion reagent, a buffer reagent, antifoam reagent, or combination thereof).

Continuing with method 400, second determination step 440 includes electrochemically determining cobalt that is free of albumin complexes in the second mixture. The result of determination step 440 is an electrochemical signal related to the amount (e.g., concentration) of cobalt that is free of albumin in the second mixture. The electrochemical determination of step 440 is performed using the same electrochemical technique used to perform determination step 420. In an exemplary embodiment, determination step 440 is performed amperometrically as discussed for step 120. For example, free cobalt can be determined in the presence of nickel in the second mixture by measuring a current resulting from a cobalt redox reaction within a potential range of between about 0.6 V and about 0.8 V. As seen in FIG. 2, an electrochemical signal y₂^{I} is obtained from a second mixture including ischemic sample material and an electrochemical signal y₂^{N} is obtained from a second mixture including non-ischemic sample material.

Continuing with method 400, assay result determination step 450 includes determining an assay result based at least in part on the electrochemical signals of first and second determining steps 420,440. In some embodiments, determination step 450 includes determining a slope and intercept defined by the results of first and second determination steps 420,440. For example, the intercept b₁^{I} results from electrochemical signals y₁^{I} and y₂^{I} (FIG. 2, curve 168). The intercept b₁^{N} results from electrochemical signals y₁^{N} and y₂^{N} (FIG. 2, curve 162).

The assay result of determining step 450 can be used to determine whether the human from which the sample material was obtained has experienced or is experiencing an ischemic event. For example, the presence of an ischemic event can be determined by comparing an intercept determined using sample material (e.g., blood such as whole blood) with a reference value. An exemplary reference value is an intercept determined using the same type of sample material obtained from one or more subjects known to have experienced or be experiencing an ischemic event. The presence of an ischemic event in a subject is diagnosed if the intercept determined by applying method 400 to sample material obtained from the subject falls below the corresponding reference difference value. For example, intercept b₁^{I} determined from ischemic sample material falls below intercept b₁^{N}determined using non-ischemic sample material.

The intercepts b₁^{I} and b₁^{N} correspond to the difference values 176,174 determined according to assay result determination step 150. For example, difference value 176 (FIG. 2) is given by y₂−y₁^{I}=m([Co]¹−[Co])+b₂−b₁^{I} (Eq. 4), where [Co]₁ is the cobalt concentration in the first and second mixtures. Difference value 176 is about equal to b₁^{I} where b₂ is close to zero. Similarly, difference value 174 is about equal to b₁^{N} where b₂ is close to zero.

Because assay result of determining step 450 includes determining a result based on the combination of determining steps 420,440, step 450 shares the advantages of being less susceptible to matrix effects than if the assay result was based on the result of first determining step 420 alone. Accordingly, a determination of ischemia based on method 400 is also less susceptible to matrix effects than if the determination were made based on the result of first determining step 420 alone.

While method 400 has been described using whole blood, other sample materials can be used. For example, the sample material can alternatively be or additionally include a blood derived material (e.g., plasma, serum, or combination thereof). As describe for method 100, when a material that lacks red blood cells (e.g., plasma or serum) is substituted for (or combined with) whole blood, greater the amounts of reagents are typically used to compensate for the greater available volume resulting from the lack of red blood cells.

We now describe devices and systems for performing assays (e.g., in accord with method 100 or 400 using a blood derived fluid (e.g., whole blood, serum, plasma, or combination thereof)).

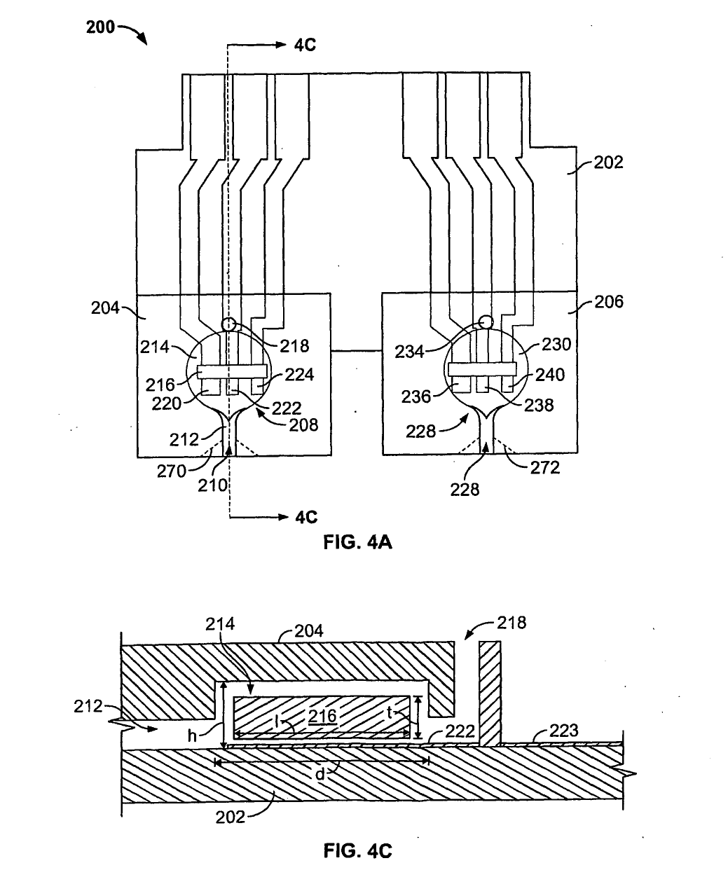

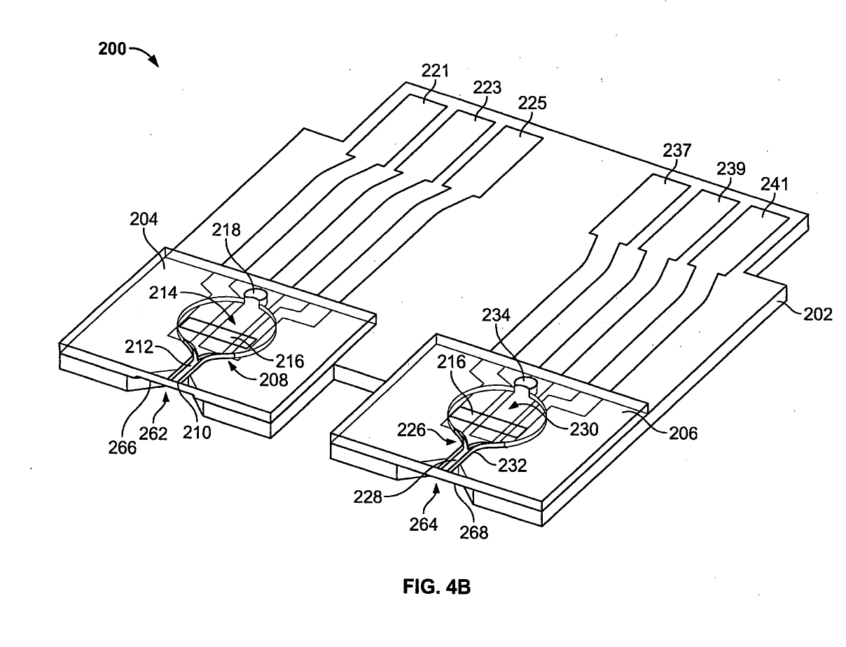

Referring to FIGS. 4a-4c, a microfluidic device 200 includes a major substrate 202 and two secondary substrates 204,206. Major substrate 202 and secondary substrate 204 define a first microfluidic network 208 including a sample material inlet 210 and a first detection zone 214 connected to inlet 210 by a channel 212. First microfluidic network 208 is configured to receive sample material and form a reagent-sample material mixture (e.g., in accord with first mixture formation step 110 of method 100). First detection zone 214 includes reagent material (not shown) including a cobalt reagent, a stir bar 216, a vent 218, and first, second, and third electrodes 220,222,224, which are respectively connected to first, second, and third leads 221,223,225. Major substrate 202 and secondary substrate 206 define a second microfluidic network 226 including a sample material inlet 228 and a second detection zone 230 connected to inlet 228 by a channel 232. Second microfluidic network 226 is configured to receive a sample and form a reagent-sample material mixture (e.g., in accord with second mixture formation step 130 of method 100). Second detection zone 230 includes reagent material (not shown) including a cobalt reagent and a nickel reagent, a stir bar 216, a vent 234, and first, second, and third electrodes 236,238,240 which are respectively connected to first, second, and third leads 237,239,241.

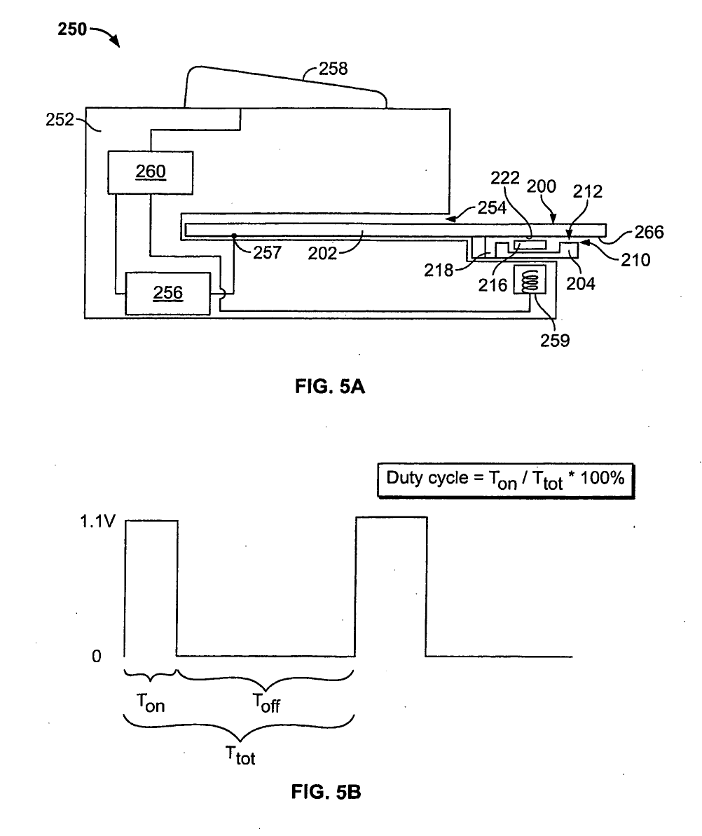

Referring to FIG. 5a, an assay system 250 includes an assay reader 252 and assay device 200. Assay reader 252 includes an entry port 254, an electrochemical detector 256, a display 258, a magnetic stir bar actuator 259, and a processor 260 typically in communication with detector 256, stir bar actuator 259, and/or display 258. In use, device 200 is received within entry port 254 of reader 252. Stir bar actuator 259 moves (e.g., rotates) stir bars 216 within detection zones 214,230 to assist formation of respective sample material-reagent mixtures therein. Electrochemical detector 256 includes respective contacts 257 (only one of which is shown) to operate electrodes 220,222,224 via leads 221,223,225 and electrodes 236,238,240 via leads 237,239,241. Processor 260 receives electrochemical signals from electrochemical detector 256 and determines an assay result based on the signals. Display 258 displays the assay results and/or a determination of the presence of an ischemic condition based on the results.

Inlets 210,228 are each proximate a respective sample receiving zone 262,264. Each zone 262,264 is defined by a respective sample material receiving surface 266,268 of secondary substrates 204,206 a respective notch 270,272 in major substrate 202. In use, a respective amount of sample material (e.g., blood derived material such as whole blood) is introduced to each sample receiving zone 262,264. Sample material receiving surfaces 266,268 support the sample and capillary action by surfaces 266,268 and walls of notches 270,272 draws the sample to inlets 210,228. Sample material that contacts inlets 210,228 flows by capillary action along respective channels 212,232 to detection zones 214,230.

Sample material (e.g., whole blood, plasma, serum, or combination thereof) applied to inlet 210 flows by capillary action into detection zone 214. Vent 218 permits gas (e.g., air) to exit detection zone 214 as sample material enters. The sample material received by detection zone 214 combines with (e.g., suspends or solubilizes) reagent material therein to form a first mixture in accord with first mixture formation step 110. Sample material (e.g., whole blood) applied to inlet 228 flows by capillary action into detection zone 230. Vent 234 permits gas (e.g., air) to exit detection zone 230 as sample material enters. The sample material received by detection zone 230 combines with (e.g., suspends or solubilizes) reagent material therein to form a second mixture in accord with second mixture formation step 130.

Sample receiving zones 262,264 and detection zones 214,230 are configured to accommodate an amount of sample material sufficient to perform an electrochemical determination (e.g., in accord with determination steps 120,140 of method 100). Typically, a free volume of each of detection zones 214,230 can be filled by an amount of sample material (e.g., blood) obtained with a small number (e.g., three or less, two or less, one) of “finger sticks”. For example, in some embodiments, the combined free volume of detection zones can be filled with blood from a single “finger stick”. The free volume of each detection zone is the volume available to be occupied by sample material and reagent material. The free volume excludes the volume of stir bar 216. In some embodiments, the combined free volume of detection zones 214,230 is about 100 μl or less (e.g., 75 μl or less, 50 μl or less, 30 μl or less). In some embodiments, the combined free volume of detection zones 214,230 is about 2.5 μl or more (e.g., about 5 μl or more, about 15 μl or more). In an exemplary embodiment, the combined free volume of detection zones 214,230 is between about 5 and about 10 μl.

Typically, the respective dimensions and volumes of detection zones 214,230 are about the same (e.g., essentially identical). In some embodiments, an average radial dimension (e.g., diameter) of each detection zone is at least about 2 mm (e.g., at least about 3 mm) and the average radial dimension (e.g., diameter) is about 7.5 mm or less (e.g., about 6 mm or less, about 5 mm or less). In an exemplary embodiment, the average radial dimension of each detection zone is about 4 mm. In some embodiments, the average heights of detection zones 214,230 are each at least about 100 μm (e.g., at least about 250 μm, at least about 300 μm) and the average heights are each about 1000 μm or less (e.g., about 750 μm or less, about 500 μm or less). In an exemplary embodiment the average heights of detection zones 214,230 are each between about 100 and 200 μm (e.g., about 150 μm to about 175 μm).

The reagent material of detection zone 214 includes a cobalt reagent that provides cobalt ions when combined with the sample material. Exemplary cobalt reagents are those discussed with respect to first mixture formation step 110 of method 100. For example, the cobalt reagent can include a solid (e.g., cobalt in the form of a cobalt salt (e.g., cobalt chloride)). The reagent may be a dry cobalt salt. Sample material solubilizes the cobalt reagent to form a mixture having properties (e.g., cobalt concentration) as discussed with respect to first mixture formation step 110 of method 100.

The amount of cobalt reagent depends on the cobalt concentration to be achieved within detection zone 214 and the free volume of the detection zone. The amount of reagent may also be varied depending upon the free volume of the sample material (e.g., depending upon the volume of sample occupied by red blood cells) as discussed for methods 100 and 400.

In some embodiments, the amount of cobalt reagent is sufficient to obtain, when solubilized, a cobalt concentration in detection zone 214 of at least about 500 μM (e.g., at least about 750 μM, at least about 1.0 mM, at least about 1.75 mM, at least about 3 mM). In some embodiments, the amount of cobalt reagent is sufficient to obtain, when solubilized, a cobalt concentration in detection zone 214 of about 15 mM or less (e.g., about 10 mM or less, about 7.5 mM or less, about 5.0 mM or less). In an exemplary embodiment, the amount of cobalt reagent is sufficient to obtain, when solubilized, a cobalt concentration in detection zone 214 of between about 0.75 mM and about 2 mM (e.g., about 1.0 mM). As discussed above, any concentration referred to herein is an “actual” concentration in which the volume is determined by including the volume occupied by red blood cells. For a detection zone essentially completely filled with sample, the volume of the mixture is the filled volume of the detection zone.

The reagent material of detection zone 214 may include any reagents discussed herein, for example, as discussed with respect to mixture formation steps 110, 130 (e.g., a chloride compensating reagent, a suspension enhancing reagent, a plasticizing reagent, a dispersion reagent, a buffer reagent, antifoam reagent, or combination thereof).

The reagent material of detection zone 230 includes a cobalt reagent that provides cobalt ions and a nickel reagent that provides nickel ions when combined with the sample material. Exemplary cobalt reagents are those discussed with respect to first mixture formation step 110 of method 100 and detection zone 214. For example, the cobalt reagent can include a solid (e.g., cobalt in the form of a cobalt salt (e.g., cobalt chloride)). The reagent may be a dry cobalt salt. Exemplary nickel reagents are those discussed with respect to second mixture formation step 130 of method 100. For example, the nickel reagent may include a solid (e.g., nickel in the form of a nickel salt (e.g., nickel chloride (NiCl₂))). In an exemplary embodiment, the reagent includes dry cobalt salt and dry nickel salt.

Sample material solubilizes the cobalt and nickel reagents to form a mixture having properties (e.g., cobalt and nickel concentrations) as discussed with respect to second mixture formation step 130 of method 100. For example, the nickel concentration in detection zone 230 is typically high enough that essentially no cobalt-albumin complexes are present (e.g., most or substantially all of the cobalt is prevented from forming cobalt-albumin complexes and/or is displaced from cobalt-albumin complexes). Typically, the nickel reagent of detection zone 230 provides a total nickel concentration that is higher than the cobalt concentration provided by the cobalt reagent. For example, a ratio of the total nickel concentration provided by the nickel reagent to cobalt concentrations provided by the cobalt reagent is typically at least about 1.5 (e.g., at least about 3, at least about 5, at least about 10). The ratio of the total nickel to cobalt concentrations provided by the nickel and cobalt reagents is typically about 25 or less (e.g., about 20 or less, about 15 or less, about 10 or less). In an exemplary embodiment, the ratio is about 5.5.

In some embodiments, the amount of nickel reagent is sufficient to provide a total nickel concentration in detection zone 230 of at least about 3 mM (e.g., at least about 5 mM, at least about 7.5 mM, at least about 10 mM, at least about 15 mM). In some embodiments, the amount of nickel reagent is sufficient to provide a total nickel concentration in detection zone 230 of about 30 mM or less (e.g., about 25 mM or less, about 20 mM or less, about 15 mM or less). In an exemplary embodiment, the amount of cobalt reagent is sufficient to provide a cobalt concentration in detection zone 230 of about 3.5 mM or less (e.g., about 1.5 mM) and the amount of nickel reagent is sufficient to provide a total nickel concentration in detection zone 230 of between about 5 mM and about 20 mM (e.g., about 7.5 mM).

As discussed with respect to detection zone 214, the reagent material of detection zone 230 may include any combination of reagents discussed herein, for example, as discussed with respect to mixture formation steps 110, 130 (e.g., a chloride compensating reagent, a suspension enhancing reagent, a plasticizing reagent, a dispersion reagent, a buffer reagent, antifoam reagent, or combination thereof).

Stir bars 216 are sized and shaped to fit within detection zones 214,230 and to assist combination of sample and reagent material within the detection zones. Typically, a ratio of a volume of stir bar 216 to a volume of a corresponding detection zone 214,230 is at least about 0.04 (e.g., at least about 0.06, at least about 0.07). The ratio of volumes can be about 0.15 or less (e.g., about 0.12 or less, about 0.1 or less). In an exemplary embodiment, the ratio of a volume of stir bar 216 to a volume of a corresponding detection zone 214,230 is between about 0.07 and 0.1 (e.g., about 0.85).

A ratio of a height of stir bar 216 to a height of a corresponding detection zone 214,230 (taken with the stir bar positioned within the detection zone) is typically at least about 0.6 (e.g., at least about 0.65, at least about 0.7). The ratio of heights is typically about 0.9 or less (e.g., about 0.8 or less). In an exemplary embodiment, the ratio of heights is between about 0.75 and about 0.8 (e.g., about 0.775).

In some embodiments, a length of stir bar 216 is between about 2 mm and about 4 mm (e.g., about 3 mm), a width of stir bar 216 is between about 0.25 mm and about 0.6 mm (e.g., about 0.45 mm), and a height of stir bar 216 is between about 0.15 mm and about 0.6 mm (e.g., about 0.3 mm). In some embodiments, a volume of stir bar 216 is between about 0.25 μl and about 0.6 μl (e.g., about 0.45 μl).

Stir bar 216 is typically coated to minimize interaction with reagent and sample material and to prevent shorting of electrodes within the detection zone. In an exemplary embodiment, the stir bar is coated with parylene. As an alternative or in addition, the stir bar may be coated with PVP.

To assist combination of sample material and reagent material within respective detection zones 214,230, stir bar actuator 259 subjects stir bars 216 to an alternating magnetic field to move (e.g., rotate) stir bar 216 within detection zones 214,230. Stir bars 216 are formed of a magnetically susceptible material (e.g., iron or an iron-containing metal). Stir bar 216 movement causes material within the detection zone to move thereby enhancing combination of the sample and reagent materials. Processor operates stir bar actuator 259 to control a speed at which stir bars 216 move. For example, stir bar actuator 259 may use a feedback loop to limit a rotational speed of stir bars 216. Limiting the rotational speed can enhance the ability of stir bars 216 to cause movement of materials within detection zones 214,230 thereby enhancing combination of the sample and reagent materials.

Stir bar actuator 259 typically includes a pair of DC motors (e.g., a 95 c motor having a size of about 10×6 mm), each having a low power requirement. An optical feedback loop is used to control the speed of the motor to reducing rotational variation. As the reagents combine with the sample, the viscosity of the mixture changes. This changes the drag on the motor caused by inductive coupling between stir bar 216 and the magnet of the motor.

During mixing, an optical system is used to determine the rotational speed of the motor. The speed is controlled by modulating the DC voltage applied to the motor, typically by varying the duty cycle of the voltage applied to the motor. Referring to FIG. 5b, the duration of Ton can be modulated to achieve the desired speed. Reader 252 allows real-time feedback control of the duty cycle to achieve the desired speed with negligible over shoot. Thus by applying the control algorithm to the motor, which by default would rotate at a high rate (e.g., about 10,000 rpm), the rotational speed can be controlled. In an exemplary embodiment, the rotational speed is controlled to between about 250 and about 1500 rpm (e.g., between about 480 and about 1250 rpm, e.g., about 1000 rpm). Mixing is performed for a period of time (e.g., between about 60 and 120 seconds, such as about 90 seconds).

Reader 252 operates electrodes 220,222,224 to determine the amount of free cobalt in the mixture formed in detection zone 214 in accord with first determination step 120 of method 100. Processor 252 also operates electrodes 236,238,240 to determine the amount of free cobalt in the mixture formed in detection zone 230 in accord with second determination step 140 of method 100.

Device 200 can be manufactured as desired. Substrates 202,204,206 can be made by any desired method, such as, for example, injection molding. Exemplary substrate materials include polymers such as injection moldable polymers. An exemplary polymer is polyester. Other suitable materials include alumina ceramic, glass (e.g., fused silica), quartz, and silicon. The substrate material can be coated to modify its hydrophobicity (e.g., to render it more hydrophilic).

Electrodes and leads are typically formed on major substrate 202. Electrodes and leads can be deposited by screen printing on the substrate with a carbon ink, silver ink, or gold ink. Examples of suitable inks (e.g., pastes) include carbon D2 paste (GEM Ltd) and silver|silver chloride paste (DuPont). Typically, a carbon paste is used for a working electrode and a counter electrode. A silver|silver chloride paste is typically used for a reference electrode. The electrodes and/or leads can be covered at least in part with an insulation layer. An exemplary insulation layer is formed from a dielectric paste such as dielectric D1 paste (GEM Ltd). Electrodes and leads can also be formed by evaporation or sputtering of a conductive material (such as, for example, gold, silver or aluminum) on the base, followed by laser ablation or photolithographic masking and a wet or dry etch.

Features of secondary substrates 204,206 that form microfluidic networks when mated with major substrate 202 can be formed as desired. Such features include grooves that form channels 212,232, recesses that form detection zones 214,230, and passages that form vents 218,234. In an exemplary embodiment, secondary substrates 204,206 are formed by injection molding including most (e.g., all of such features). Other techniques can also be used to form such features. For example, features can be formed by laser ablation, photolithography with etching, imprinting, or micromachining.

Reagent material can be deposited as desired. In some embodiments, reagent materials are deposited into recesses of the secondary substrates prior to mating with major substrate 202. Deposition of reagents can be accomplished, for example, by dispensing or aspirating from a nozzle, using an electromagnetic valve and servo- or stepper-driven syringe.

These methods can deposit droplets or lines of reagents in a contact or non-contact mode. Reagent material is typically deposited on a substrate that forms part of a detection zone in a complete device. For example, reagent material can be deposited in multiple droplets (e.g., at least 3, at least 5, at least about 10 droplets). In some embodiments, reagent material is deposited in 50 or fewer droplets (e.g., 25 or fewer droplets).

Other methods for depositing reagents include pad printing, screen printing, piezoelectric print head (e.g., ink-jet printing), or depositing from a pouch which is compressed to release reagent (a “cake icer”). Deposition can preferably be performed in a humidity- and temperature-controlled environment. Different reagents can be dispensed at the same or at a different station.

In some embodiments, reagents are screen printed. For example, reagent materials can be screen printed on a surface of a substrate of the device. In some embodiments, reagents are deposited (e.g., printed) directly over a surface of electrodes. In some embodiments, reagents are deposited (e.g., printed) on a surface of a substrate that opposes the electrodes in the completed device.

Typically, the volume of reagent material (prior to drying) applied to the detection zone is less than the volume of the detection zone. In some embodiments, the volume of applied reagent material is about 50% or less (e.g., about 25% or less, about 20% or less, about 15% or less) of the volume of the detection zone. In some embodiments, the volume of applied material (prior to drying) is about 5% or more (e.g., about 7.5% or more, about 10% or more) of the volume of the detection zone. In exemplary embodiments, a volume (prior to drying) of between about 0.5 μl and about 5 μl (e.g., between about 1 μl and about 2 μl) of reagent material is applied. The cobalt and/or nickel of the applied reagent material typically has a concentration given by the desired concentration of the cobalt or nickel in the reagent sample material mixture formed in the detection zone during use (e.g., about 1.75 mM Co) multiplied by the ratio of the detection zone volume (e.g., about 5 μl) to the applied reagent material volume (e.g., about 1 μl): 1.75 mM Co (5 μl/1 μl)=8.75 mM Co.

The reagent material applied may be an aqueous solution or it can include one or more organic solvents (e.g., acetone, ethanol, or combination thereof).

Fluorescent or colored additives can optionally be added to the reagents to allow detection of cross contamination or overspill of the reagents outside the desired deposition zone. Product performance can be impaired by cross-contamination. Deposition zones can be in close proximity or a distance apart. The fluorescent or colored additives are selected so as not to interfere with the operation of the assay device, particularly with detection of the analyte.

After deposition, the reagents are dried. Drying can be achieved by ambient air drying, infrared drying, infrared drying assisted by forced air, ultraviolet light drying, forced warm, controlled relative humidity drying, or a combination of these.

Stir bars 216 may be deposited into recesses of secondary substrates 204,206 prior to or after deposition of reagent materials as just described. Reagent material can be used to immobilize the stir bar within the detection zone. For example, the stir bar can be contacted with reagent material deposited in the recess of the substrate while the reagent material is still moist. The immobilization is typically to the extent that the stir bar can be mechanically actuated to assist solubilization of the reagent

The reagent material is soluble to the extent that sample material which enters the detection zone solubilizes the reagent material thereby allowing the stir bar to rotate and mechanically assist solubilization of the reagent material. Typically, the reagent material includes a plasticizing reagent such as a cellulose derivative (e.g., a hydroxyethylcellulose such as Natrosol G, Agualon). In some embodiments, the stir bar is immobilized so that a long axis of the stir bar is at a non-zero angle (e.g., at least about 45°, at least about 70°, at least about 80°, about perpendicular to) with respect to a flow of sample material entering the detection zone. For example, the long axis of the stir bar may be at such a non-zero angle with respect to a longitudinal axis of a channel by which sample material enters the detection zone. Typically, the stir bar is immobilized so that the stir bar does not touch a side wall of the detection zone.

Secondary substrates 204,206 are mated with corresponding portions of major substrate 202 to form the microfluidic networks. Mating can be accomplished using, for example, an adhesive backed laminate disposed between the secondary and major substrates.

In some embodiments, reagent material deposition is performed after mating the second and major substrates. Post-mating deposition can be accomplished by, for example, at least partially (e.g., completely) filling the detection volumes with a reagent to material solution (e.g., a cobalt solution or a cobalt/nickel solution), followed by drying (e.g., by freeze drying). If the detection volumes or completely filled with reagent material solution, the ratio of the mass of deposited reagent material to the detection zone volume is constant independent of any variation in detection zone volume.

While assay device 200 and assay reader 252 have been exemplified as performing an assay in accord with method 100, the device and assay reader may be configured to perform other assays. For example, the device and reader can be configured to perform an assay in accord with method 400. In such embodiments, the first detection zone of the device may have the same properties (e.g., size and reagent composition) as detection zone 214 of device 200. The second detection zone may have the same size as detection zone 230 of device 200 but includes a reagent composition as described for step 430 of method 400 including, for example, a chloride compensating reagent, a suspension enhancing reagent, a plasticizing reagent, a dispersion reagent, a buffer reagent, antifoam reagent, or combination thereof.

The reader is configured to operate the device in accord with method 400 optionally including the determination of an ischemic event.

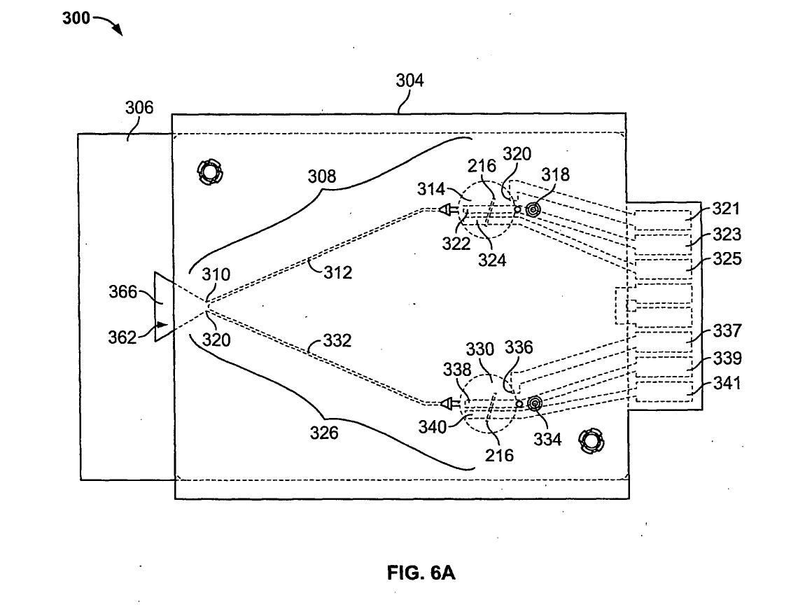

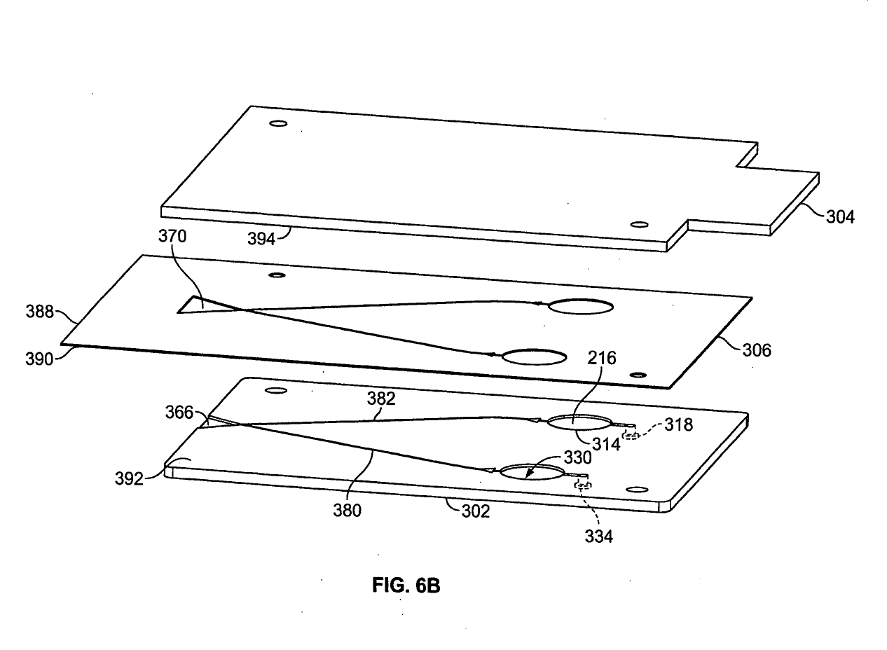

While a microfluidic device has been described to perform determinations on each of two separate portions of sample material, devices can have other configurations. Referring to FIGS. 6a and 6b, a device 300 is configured to perform multiple determinations using a single portion of sample material (e.g., in accord with method 100 or 400). Device 300 can be operated using assay reader 252 as described for operation of device 200 using assay system 250 (e.g., in accord with method 100 or 400).

Device 300 includes first and second outer substrates 302,304 and an optional intermediate substrate 306. Substrates 302,304,306 define first and second microfluidic networks 308,326. First microfluidic network 308 includes a sample material inlet 310 and a first detection zone 314 connected to inlet 310 by a channel 312. First microfluidic network 308 is configured to receive sample material and form a reagent-sample material mixture (e.g., in accord with first mixture formation step 110 of method 100). First detection zone 314 includes reagent material (not shown) including a cobalt reagent, a stir bar 216, a vent 318, and first, second, and third electrodes 320,322,324, which are respectively connected to first, second, and third leads 321,323,325. Second microfluidic network 326 includes a second detection zone 330 connected to an inlet 320 by a channel 332. Second microfluidic network 326 is configured to receive a sample and form a reagent-sample material mixture (e.g., in accord with second mixture formation step 130 of method 100). Second detection zone 330 includes reagent material (not shown) including a cobalt reagent and a nickel reagent, a stir bar 216, a vent 334, and first, second, and third electrodes 336,338,340 which are respectively connected to first, second, and third leads 337,339,341.

First outer substrate 302 includes features corresponding to microfluidic networks 308,326. For example, grooves 380,382 (FIG. 6b) respectively Correspond to channels 312,332 (FIG. 6a) of microfluidic networks 308,326 and recesses 384,386 (FIG. 6b) respectively correspond to detection zones 314,330 (FIG. 6a) of microfluidic networks 308,326. Typically, substrate 302 is formed of a polymer and can be fabricated as desired (e.g., by injection molding).

In device 300, intermediate substrate 306 is sandwiched between first and second outer substrates 302,204. Substrate 306 includes first and second adhesive surfaces 388,390 which respectively mate with inner surfaces 392,394 of respective substrates 302,304. Intermediate substrate 306 includes features (e.g., cutouts) corresponding to first and second microfluidic networks 308,326.

Second outer substrate 304 cooperates with first outer substrate 302 to seal microfluidic networks 308,326. Second outer substrate 304 supports electrodes 320,322,324, 336,338,340 and leads 321,323,325, 337,339,341. The electrodes and leads can be formed as desired (e.g., by screen printing, photolithography, and laser ablation).

Inlet 310 is proximate a sample receiving zone 362 defined by a sample material receiving surface 366 of outer substrate 302 an opening 370 in intermediate substrate 306. In use, a respective amount of sample material (e.g., blood derived material such as whole blood, plasma, serum, or combination thereof) is introduced to sample receiving zone 362. Sample material receiving surface 366 supports the sample and capillary action within receiving zone 362 draws the sample to inlets 310,328 as discussed for receiving zones 262,264. Sample material that contacts inlets 310,328 flows by capillary action along respective channels 312,332 to detection zones 314,330.

Sample material (e.g., blood derived material) applied to inlet 310 flows by capillary action into detection zone 314. Vent 318 permits gas (e.g., air) to exit detection zone 314 as sample material enters. The sample material received by detection zone 314 combines with (e.g., suspends or solubilizes) reagent material therein to form a first mixture in accord with first mixture formation step 110. Sample material (e.g., whole blood) applied to inlet 328 flows by capillary action into detection zone 330. Vent 334 permits gas (e.g., air) to exit detection zone 330 as sample material enters. The sample material received by detection zone 330 combines with (e.g., suspends or solubilizes) reagent material therein to form a second mixture in accord with second mixture formation step 130.

Sample receiving zone 362 and detection zones 314,330 are configured to accommodate an amount of sample material sufficient to perform an electrochemical determination (e.g., in accord with determination steps 120,140 of method 100). Typically, a free volume of each of detection zones 314,330 can be filled by an amount of blood obtained with a small number (e.g., three or less, two or less, one) of “finger sticks”. For example, in some embodiments, the combined free volume of detection zones 314,330 can be filled with blood from a single “finger stick”. The free volume of each detection zone is the volume available to be occupied by sample material and reagent material. The free volume excludes the volume of stir bar 216. In some embodiments, the combined free volume of detection zones 314,330 is about 100 μl or less (e.g., 7 μl or less, 50 μl or less, 30 μl or less). In some embodiments, the combined free volume of detection zones is about 5 μl or more (e.g., about 10 μl or more, about 15 μl or more). In an exemplary embodiment, the combined free volume of detection zones 314,330 is about 20 μl. Typically, the respective dimensions and volumes of detection zones 314,330 are about the same (e.g., essentially identical).

The reagent material of detection zone 314 includes a cobalt reagent that provides cobalt ions when combined with the sample material. The composition and type of reagent material of detection zone 314 may be the same as described for detection zone 214 and with respect to first mixture formation step 110 of method 100. For example, the cobalt reagent can include a solid (e.g., cobalt in the form of a cobalt salt (e.g., cobalt chloride)). The reagent may be a dry cobalt salt. Sample material solubilizes the cobalt reagent to form a mixture having properties (e.g., cobalt concentration) as discussed with respect to first mixture formation step 110 of method 100.

The amount of cobalt reagent depends on the cobalt concentration to be achieved within detection zone 314 and the free volume of the detection zone. The amount of cobalt reagent may be the same as discussed for detection zone 214. For example, in an exemplary embodiment, the amount of cobalt reagent is sufficient to obtain, when solubilized, a cobalt concentration in detection zone 314 of about 3.5 mM or less (e.g., about 1.5 mM).

The reagent material of detection zone 314 may include any of the reagents discussed herein, for example, reagents as discussed with respect to mixture formation steps 110, 130 (e.g., a chloride compensating reagent, a suspension enhancing reagent, a plasticizing reagent, a dispersion reagent, a buffer reagent, antifoam reagent, or combination thereof).

The reagent material of detection zone 330 includes a cobalt reagent that provides cobalt ions and a nickel reagent that provides nickel ions when combined with the sample material. The reagent material of detection zone 330 may be, for example, any reagent material discussed with respect to detection zone 230 of device 200. Exemplary cobalt reagents are those discussed with respect to first mixture formation step 110 of method 100 and detection zones 214,314. For example, the cobalt reagent can include a solid (e.g., cobalt in the form of a cobalt salt (e.g., cobalt chloride)). The reagent may be a dry cobalt salt. Exemplary nickel reagents are those discussed with respect to detection zone 230 and second mixture formation step 130 of method 100. For example, the nickel reagent may include a solid (e.g., nickel in the form of a nickel salt (e.g., nickel chloride (NiCl₂))). In an exemplary embodiment, the reagent includes dry cobalt salt and dry nickel salt.

Sample material solubilizes the cobalt and nickel reagents to form a mixture having properties (e.g., cobalt and nickel concentrations) as discussed with respect to detection zone 230 and second mixture formation step 130 of method 100. For example, the nickel concentration in detection zone 330 is typically high enough that essentially no cobalt-albumin complexes are present (e.g., most or substantially all of the cobalt is prevented from forming cobalt-albumin complexes and/or is displaced from cobalt-albumin complexes). Typically, the nickel reagent of detection zone 330 provides a total nickel concentration that is higher than the cobalt concentration provided by the cobalt reagent. For example, a ratio of the total nickel concentration provided by the nickel reagent to cobalt concentrations provided by the cobalt reagent is typically at least about 1.5 (e.g., at least about 3, at least about 5, at least about 10). The ratio of the total nickel to cobalt concentrations provided by the nickel and cobalt reagents is typically about 25 or less (e.g., about 20 or less, about 15 or less, about 10 or less). In an exemplary embodiment, the ratio is about 5.5.

The amount of nickel reagent in detection zone 330 can be the same as discussed for detection zone 230 of device 200. For example, in an exemplary embodiment, the amount of cobalt reagent is sufficient to provide a cobalt concentration in detection zone 330 of about 3.5 mM or less (e.g., about 1.5 mM) and the amount of nickel reagent is sufficient to provide a total nickel concentration in detection zone 330 of between about 5 mM and about 20 mM (e.g., about 7.5 mM).

As discussed with respect to detection zones 230, the reagent material of detection zone 330 may include any of the reagents discussed herein, for example, reagents as discussed with respect to mixture formation steps 110, 130 (e.g., a chloride compensating reagent, a suspension enhancing reagent, a plasticizing reagent, a dispersion reagent, a buffer reagent, antifoam reagent, or combination thereof).

To assist combination of sample material and reagent material within respective detection zones 314,330, stir bars 216 can be used to move material within the detection zones as described for system 252.

While assay device 300 has been exemplified as performing an assay in accord with method 100, the device may be configured to perform other assays. For example, the device can be configured to perform an assay in accord with method 400. In such embodiments, the first detection zone of the device may have the same properties (e.g., size and reagent composition) as detection zone 314 of device 300. The second detection zone may have the same size as detection zone 330 of device 300 but includes a reagent composition as described for step 430 of method 400, including, for example, any of the reagents discussed herein, for example, reagents as discussed with respect to mixture formation steps 110, 130 (e.g., a chloride compensating reagent, a suspension enhancing reagent, a plasticizing reagent, a dispersion reagent, a buffer reagent, antifoam reagent, or combination thereof).

The reader is configured to operate the device in accord with method 400 optionally including the determination of an ischemic event.

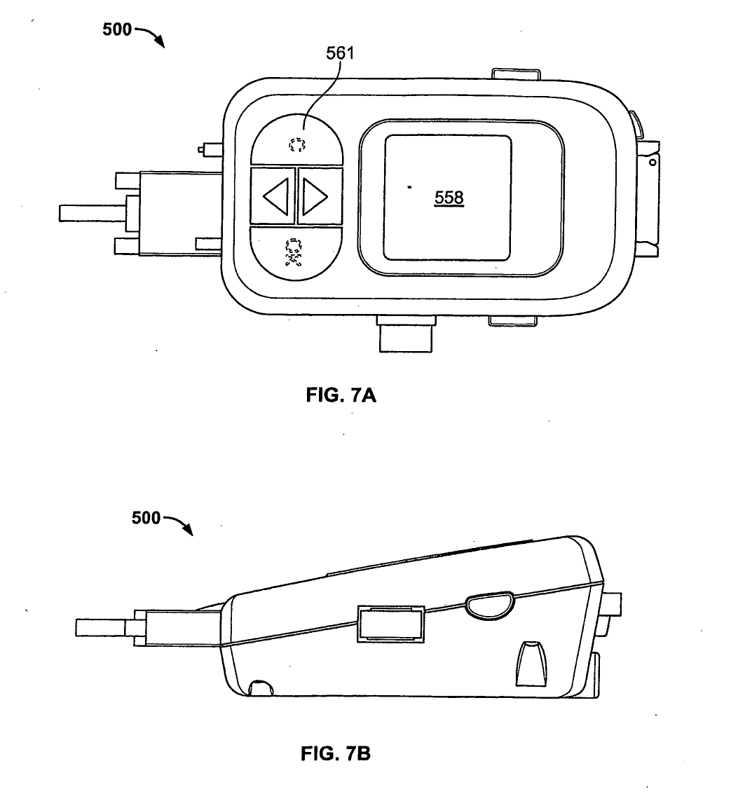

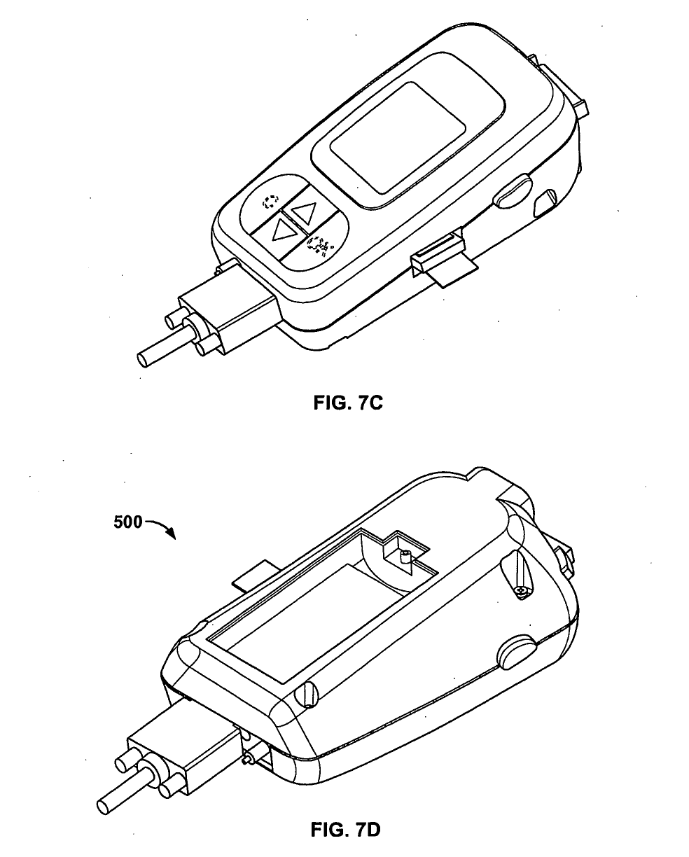

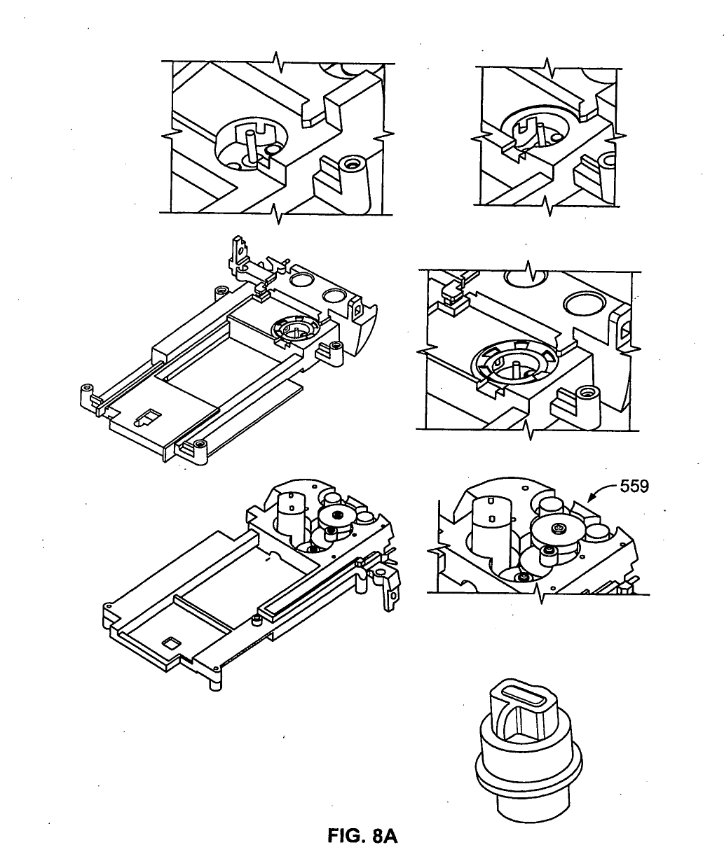

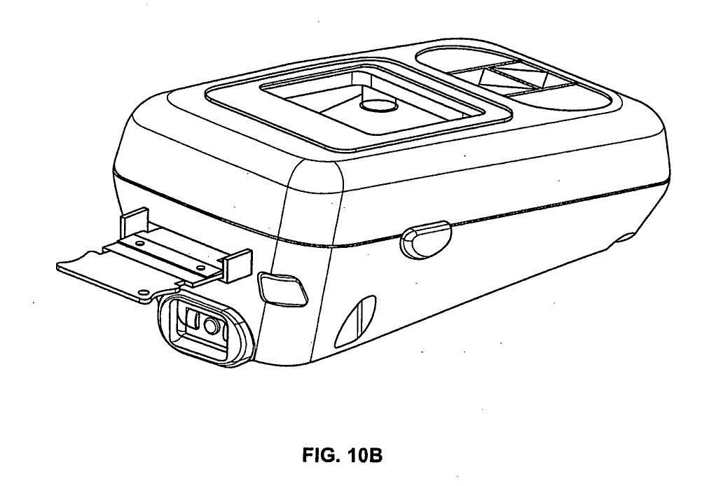

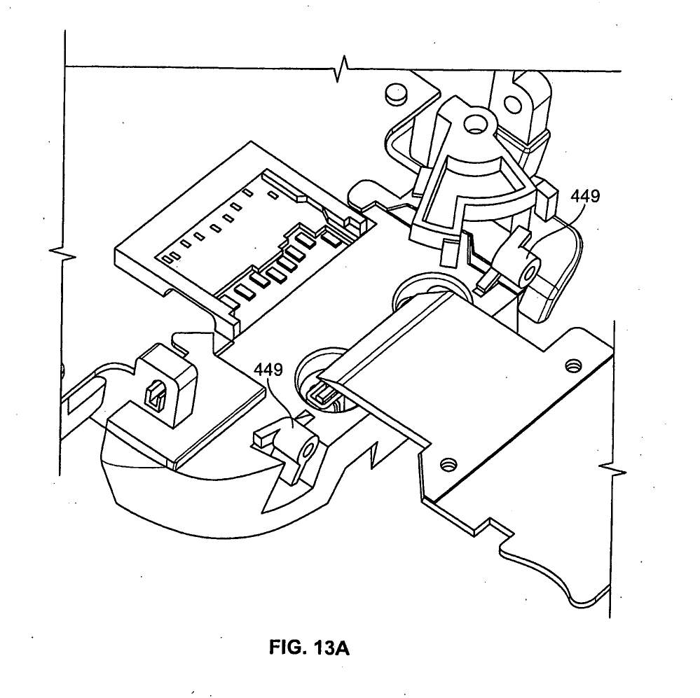

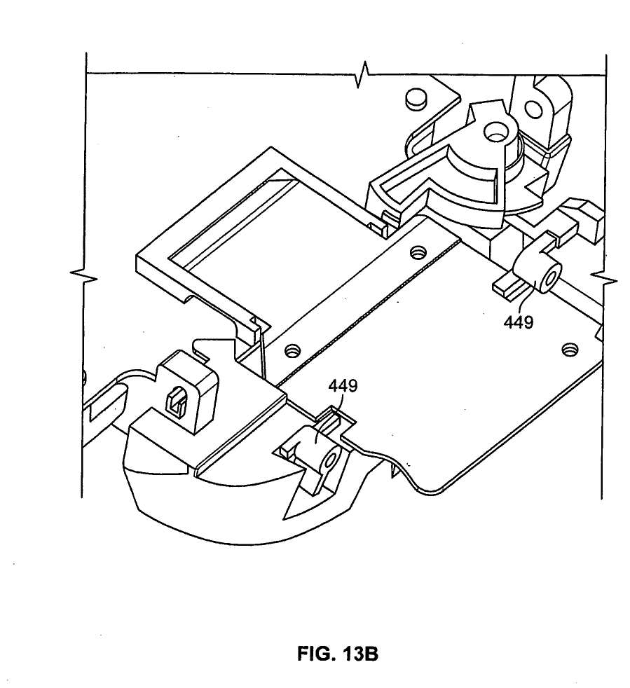

Referring to FIGS. 7a-15 an assay reader 500 is configured to operate assay devices to perform an assay. For example, assay reader can operate device 200 or device 300 in accord with method 100 or method 400.

Typically, reader 500 includes an entry port 554, an electrochemical detector, a display 558, a magnetic stir bar actuator 559, control panel 561, and a processor typically in communication with the detector, stir bar actuator, and/or the display. In use, an assay device (e.g., device 200 or 400) is received within entry port 554 of reader 552. Stir bar actuator 559 moves (e.g., rotates) stir bars 216 within detection zones of the assay device to assist formation of respective sample material-reagent mixtures therein. The electrochemical detector includes respective contacts not shown to operate electrodes of the assay device. The processor receives electrochemical signals from the electrochemical detector and determines an assay result based on the signals. Display 558 displays the assay results and/or a determination of the presence of an ischemic condition based on the results.

The magnetic stir bar actuator includes a gear driven system having a single motor that drives each of two rotors. Thus each rotor rotates at the same speed. The gear system reduces the likelihood that viscosity / drag effects of solution to be mixed will impact stir rate. A pinion gear with integrated encoder disk (a number of uniformly spaced holes around perimeter) is used to monitor rotation speed of mixer and enable feedback control of rotor speed.

Reader 500 includes a heater mechanism configured to control a temperature of the contents (e.g., mixtures of sample material and reagents) of the detection zones of an assay strip during an assay. Typically, the heater mechanism is configured to establish a temperature suitable for performing the assay. For blood-derived samples, the temperature is typically between about 35° and 40° C. (e.g., about 37° C.).

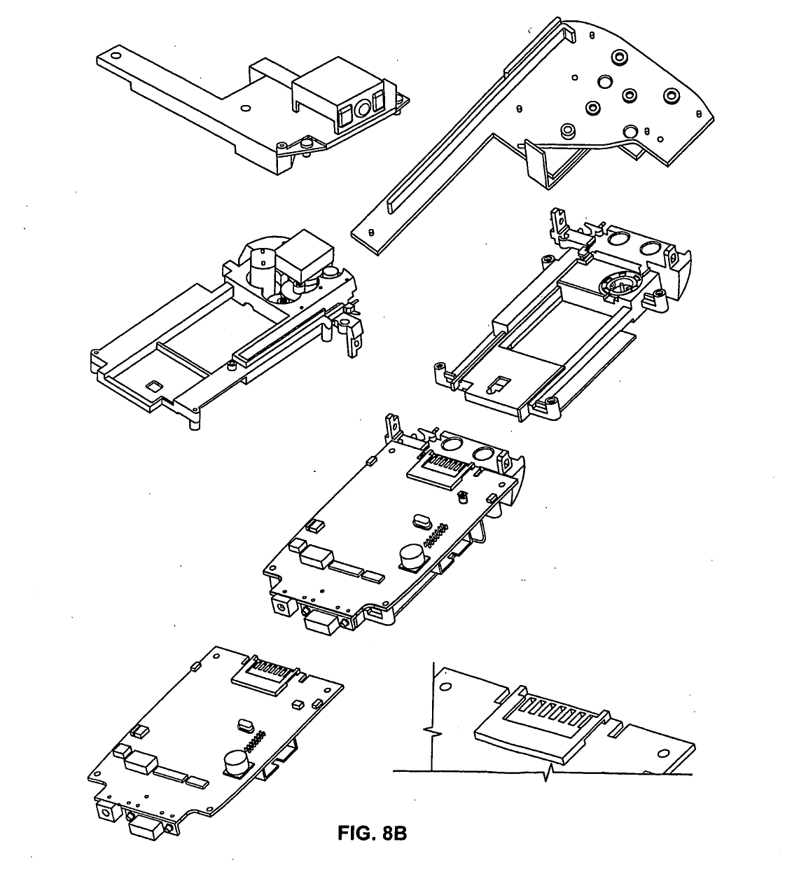

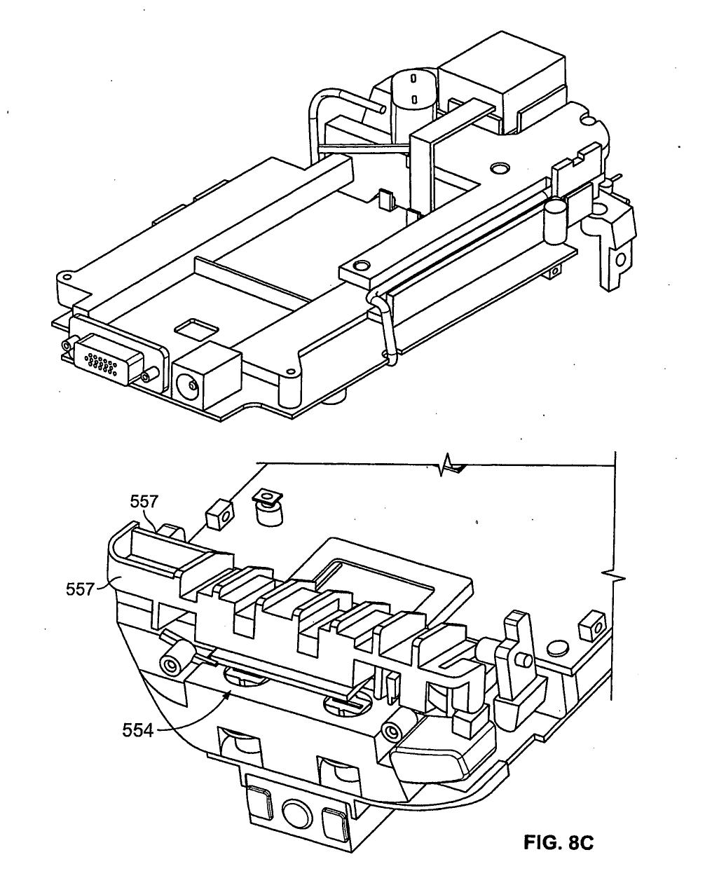

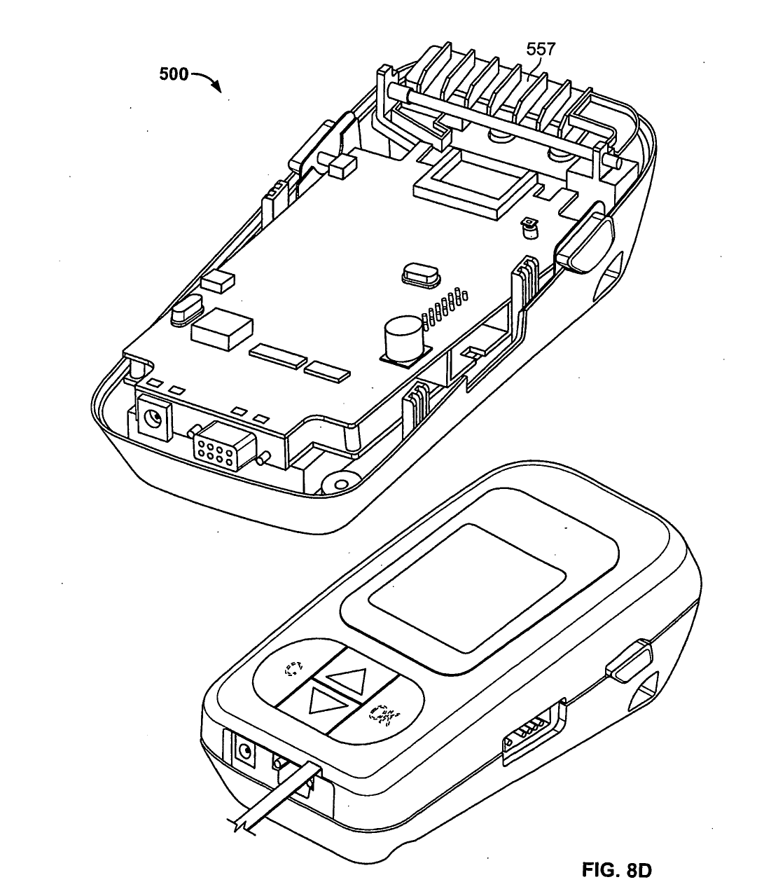

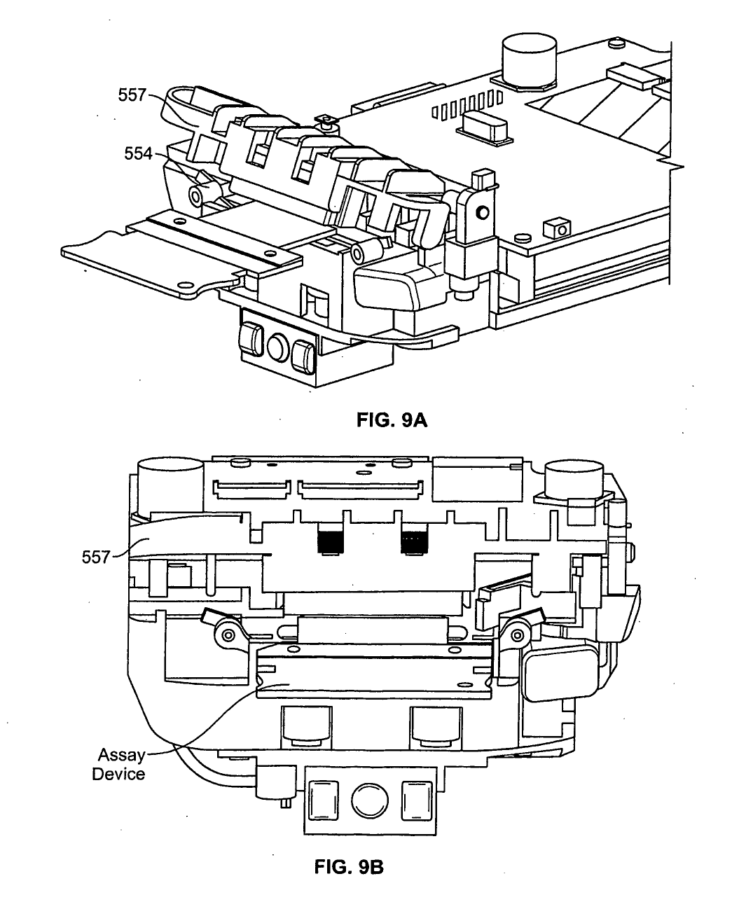

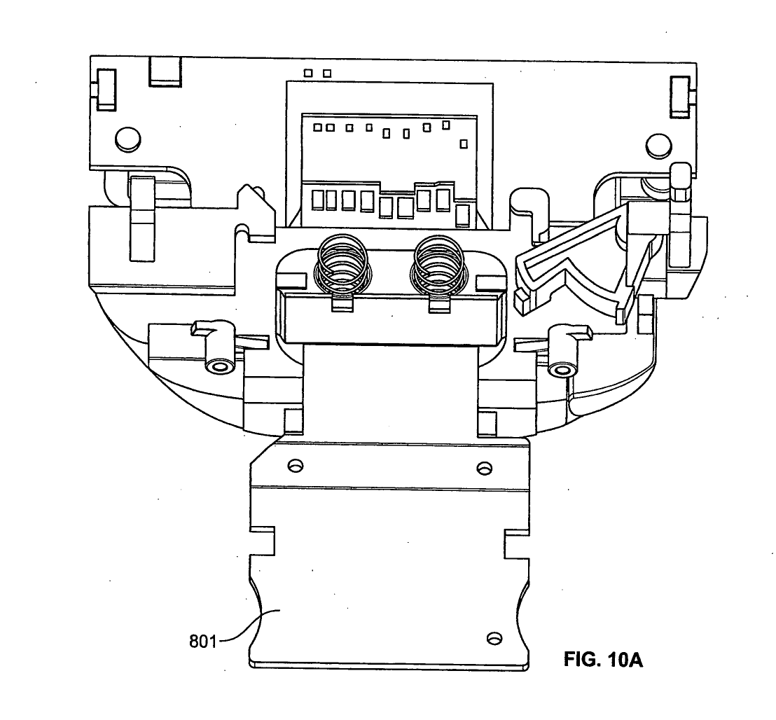

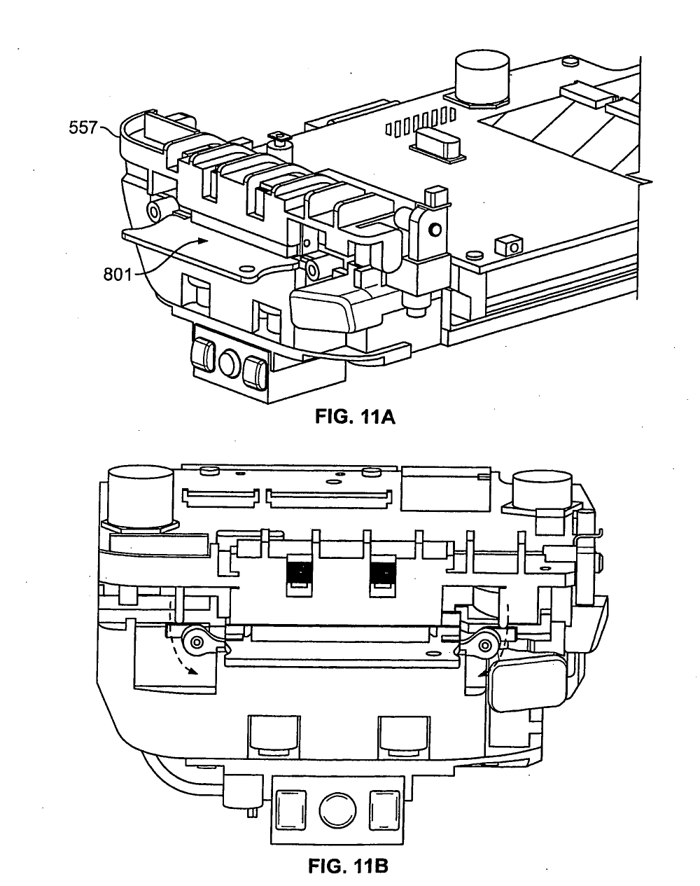

The heater mechanism includes a thermal block 557 configured to be in thermal contact (e.g., direct physical contact) with an assay device received by reader 500. Typically, the thermal block is configured to move into (e.g., rotate) into thermal contact (e.g., direct physical contact) with the assay device upon its insertion in reader. Thermal block 557 can move between a receiving position (e.g., FIGS. 8c and 9a) and a thermal contact position in which thermal block 557 is in thermal contact with the assay device (e.g., FIG. 11b). The relationship between the temperature of contents of the detection zones of the assay device and the temperature of the thermal block of the heater mechanism is calibrated so that the temperature of the contents can be predicted from the temperature of the thermal block. Because the assay device and thermal block of the temperature controller are in thermal contact, the temperature controller can monitor the temperature of the heater block rather than the temperature of the assay reader.

Reader 500 can be used with a thermal control device to confirm functionality of the heater mechanism (e.g., to confirm the calibration between the assay device detection zone contents temperature and the thermal block temperature). Typically, the thermal control device has dimensions and a shape similar to the assay device to permit the control device to be received by reader 500 and permit the thermal block to be in thermal contact with the control device upon its insertion. The thermal control device includes temperature sensors (e.g., thermistors) to monitor the temperature of the control device. The temperature sensors typically communicate with contacts of the control device. The contacts of the temperature sensors are typically positioned to be in the same location as contacts of electrodes of an assay device to be used with the reader.

The position of the temperature sensors and the thermal properties of the temperature control device (e.g., its thermal conductivity and heat capacity) are selected to be similar to an assay device or have a known relationship with respect to the thermal properties of the assay device. In use, reader 500 monitors the temperature of the thermal control device as a function of the temperature of the thermal block (e.g., by scanning the temperature of the thermal block). Reader 500 determines a calibration value that calibrates the temperature of an assay device with respect to the temperature of the thermal block.

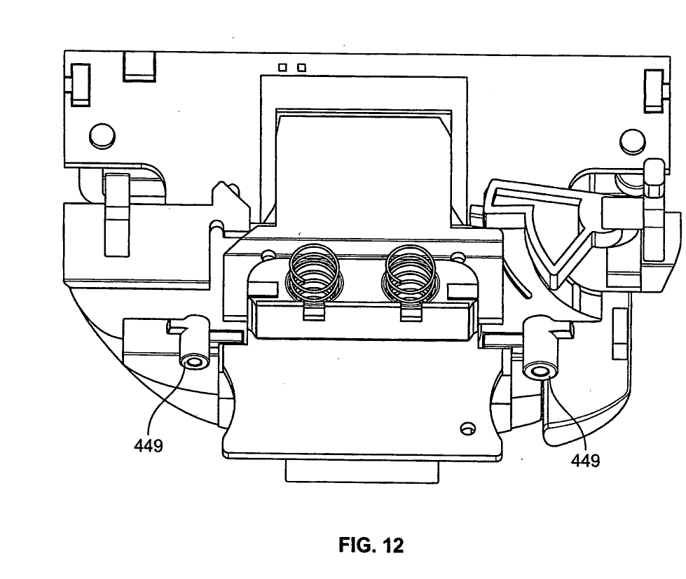

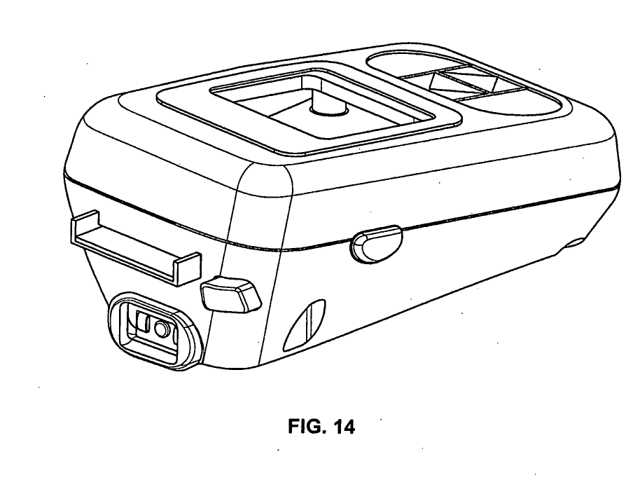

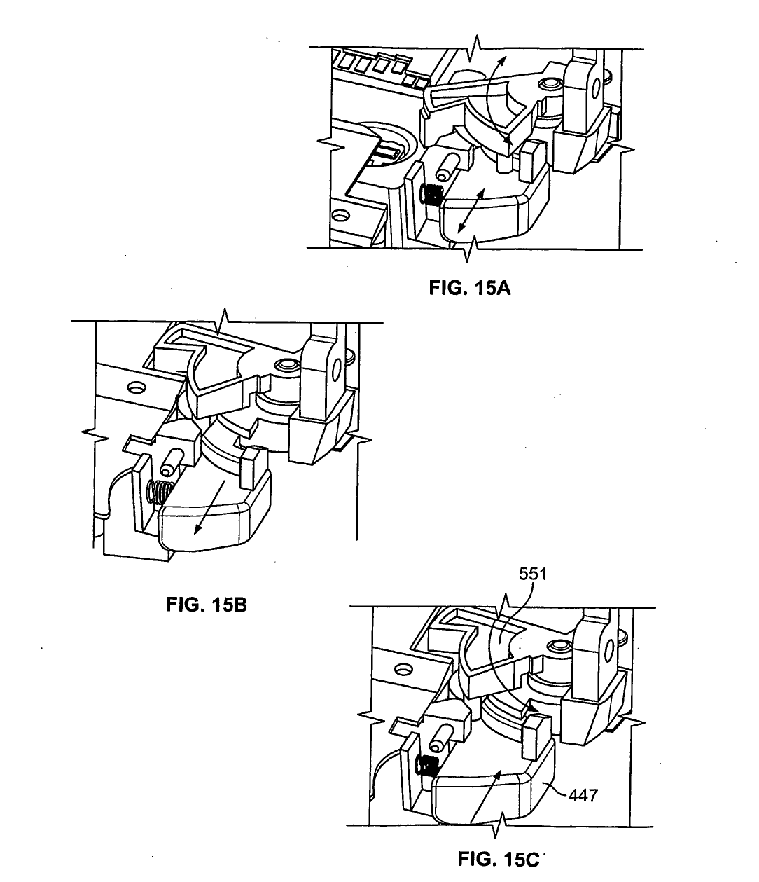

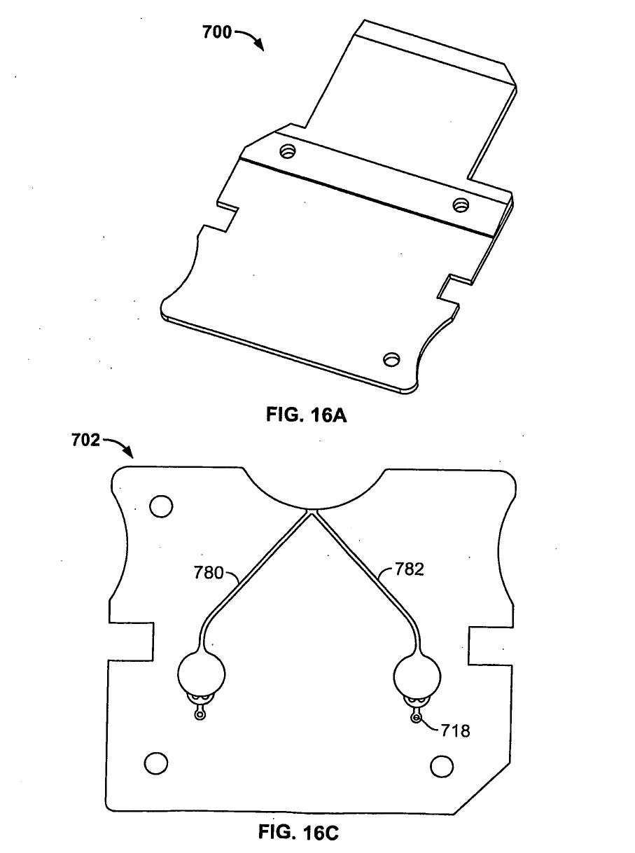

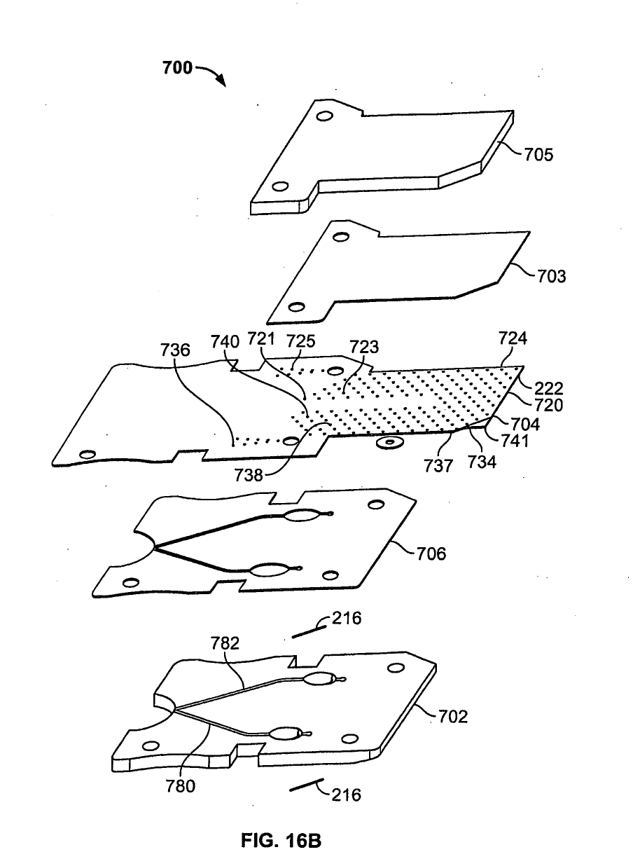

Referring to FIG. 16, an assay device 700 has a design similar to an SD Memory Card type interface. Referring also to FIGS. 12-14, assay reader 500 includes locking features 449 that engage the assay device upon its insertion into the reader to “lock” it into place. Typically, the locking features lock the assay device is the correct location with respect to the magnetic stir bar actuator to ensure effective mixing of the contents of the reaction chamber. Referring also to FIGS. 15a-15c, reader 500 includes an assay reader ejection mechanism configured to eject (e.g., at least partially remove) an assay device following an assay. The ejection mechanism includes a pushbutton 447 and a pusher 551, which operates to ejection an assay device 700 by rotating against a shoulder of the assay device.

Assay device 700 can be configured with any of the features of other assay devices discussed herein (e.g., inlets, channels, detection zones, electrodes, and/or reagent materials). In the exemplary embodiment shown, device 700 includes first and second outer substrates 702,704 and an optional intermediate substrate 706. Substrates 702,704,706 define first and second microfluidic networks as discussed for device 300. The first microfluidic network includes a sample material inlet and a first detection zone connected to the inlet by a channel. The first microfluidic network is configured to receive sample material and form a reagent-sample material mixture (e.g., in accord with first mixture formation step 110 of method 100). The first detection zone includes reagent material (not shown) including a cobalt reagent, a stir bar 216, a vent 718, and first, second, and third electrodes 721,723,725, which are respectively connected to first, second, and third leads 720,722,724. The second microfluidic network includes a second detection zone connected to an inlet by a channel. The second microfluidic network is configured to receive a sample and form a reagent-sample material mixture (e.g., in accord with second mixture formation step 130 of method 100). The second detection zone includes reagent material (not shown) including a cobalt reagent and a nickel reagent, a stir bar 216, a vent 734, and first, second, and third electrodes 336,338,340 which are respectively connected to first, second, and third leads 737,739,741 (underside of substrate 704 as shown in FIG. 16b). Device 700 can be operated by a reader in accord with method 100.

While assay device 700 has been described as being configured form mixtures and perform determinations in accord with method 100, others embodiments are possible. For example, device 700 can be configured to operate in accord with other methods discussed herein. In an exemplary embodiment, device 700 is configured to form mixtures and perform determinations in accord with method 400.

While assay devices have been described as performing a first assay on a first portion of sample and a second assay on a second portion of sample, other devices and methods can be used. For example, in some embodiments, an assay device performs a first assay (e.g., in accord with first cobalt determination step 120 of method 100 or first cobalt determination step 420 of method 400) on a sample (e.g., a blood derived sample). The device performs a second assay (e.g., in accord with second cobalt determination step 140 of method 100 or second cobalt determination step 440 of method 400) on the same portion of sample used to perform the first assay.

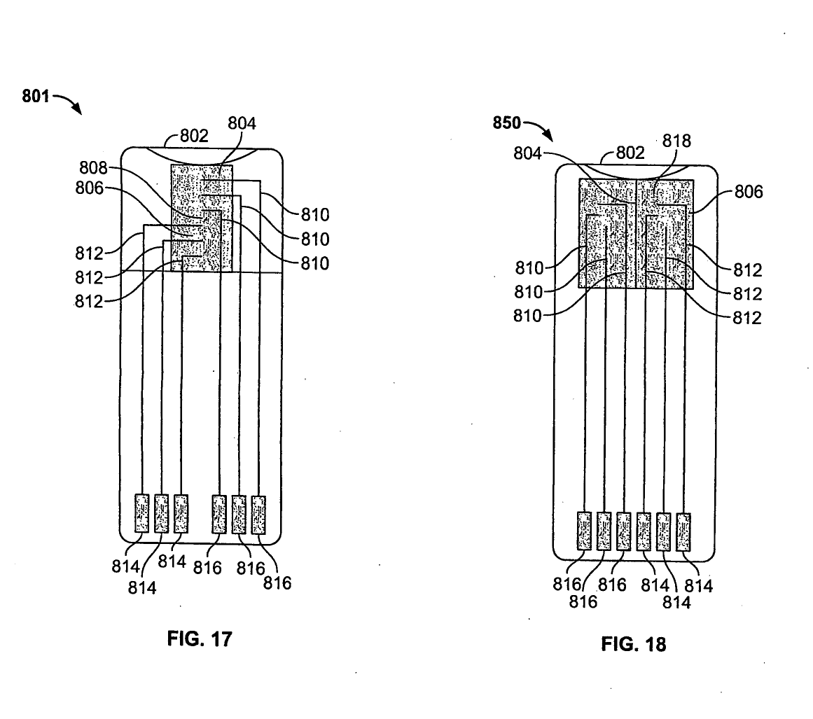

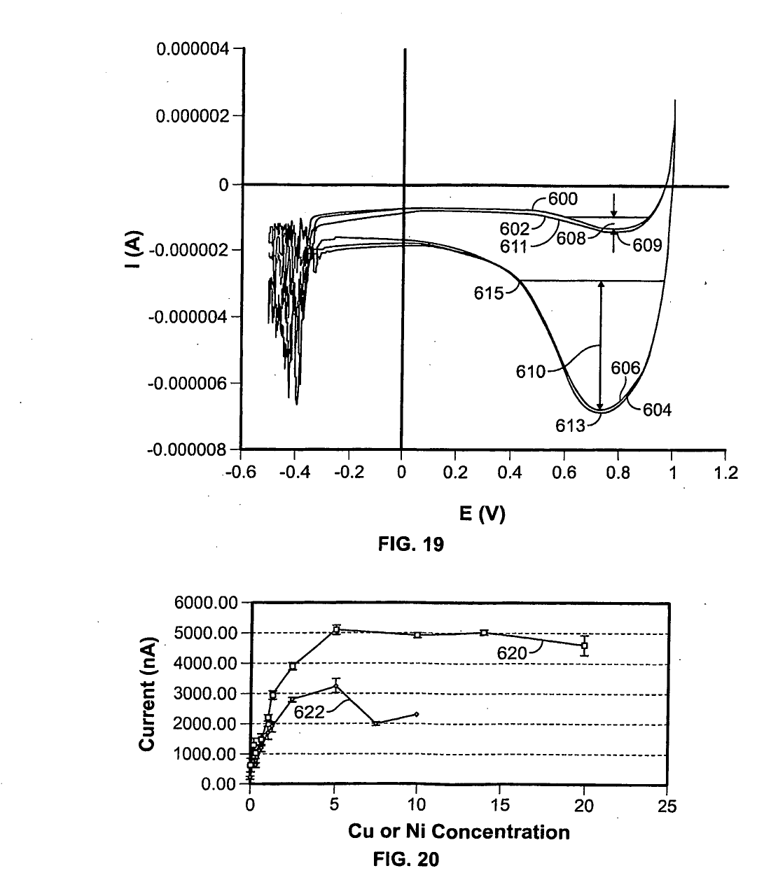

Referring to FIG. 17, an assay device 801 includes detection volumes for both a cobalt-only assay and a cobalt-and-nickel assay. Device 801 includes inlet 802, first detection volume 804 fluidly connected to the inlet, and second detection volume 806 fluidly connected to first detection volume 804 via channel 808. First detection volume 804 includes first electrodes 810 and a cobalt reagent; second detection volume 806 includes second electrodes 812, and a nickel reagent.

In use, an amount of sample material (e.g., blood derived material) is introduced to device 801 via input port 802. Device 801 is received by an assay reader. Contacts 814, 816 establish communication between the electrodes and processor of the reader via corresponding contacts (not shown) of the reader.

A first portion of blood passes along channel 808 (e.g., by capillary action) into first detection volume 804 and combines with cobalt chloride salt to form a first mixture. Albumin in the first mixture complexes a portion of the cobalt therein. The reader actuates electrodes 810 of first detection zone 804 to determine free cobalt in the first mixture. The first mixture from first detection zone 804 passes along channel 808 (e.g., by capillary action) into second detection zone 812 and combines with nickel chloride salt to form a second mixture. Nickel in the second mixture displaces cobalt from cobalt-albumin complexes that had been present in the first mixture.

The reader actuates first, second, and third electrodes 826, 828, 830 of second detection zone 818 to determine free cobalt in the second mixture. Based on results of the amount of cobalt present in the first and second mixtures, the reader determines a result indicative of the amount of albumin present in the sample material (e.g., blood) introduced to device 801 and can determine the presence of an ischemic event as described above.

Referring to FIG. 18, device 850 includes an input port 802, and a first detection zone 804. Input port 302 is fluidly connected to first detection zone 804. First detection zone 304 includes electrodes 810 in electrical communication with contacts 816. Device 850 also includes second detection zone 806 which is fluidly connected to input port 802, Second detection zone 806 includes electrodes 812 in electrical communication with contacts 814. Detection zones 804, 806 are separated by barrier 818. Barrier 818 prevents fluid exchange between detection zones 804 and 806. Barrier 818 can be formed, for example, as part of the substrate (e.g., during injection molding) or part of the lid. Barrier 818 can take the form of, for example, a wall between zones 804 and 806; or a depression (e.g. in the substrate) where the depression effectively prevents fluid transfer across the depression (for example, by disrupting capillary action):

First detection zone 804 includes a cobalt reagent; second detection zone 806 includes a cobalt reagent and a nickel reagent. In use, an amount of sample material (e.g., blood from a human) is introduced to device 801 via input port 802. Device 850 is received by an assay reader: Contacts 814, 816 establish communication between electrodes of the device and contacts of the reader. The reader actuates electrodes of the device in accord with cobalt detection steps of method 100 or 400.

We now discuss certain additional embodiments. In general, any of the methods, devices, readers, systems, reagent materials, or portions thereof as modified in accord with the following are considered part of the present disclosure.

While methods and devices have been described using blood derived sample materials, other sample materials can be used. For example, suitable sample materials include biological materials such as tissue (e.g., cardiac tissue), urine, lymph fluid, saliva, or combinations thereof. In an exemplary embodiment, the sample includes a blood derived material (e.g., blood, plasma and/or serum) or combination thereof. Other suitable sample materials include industrial and/or research materials (e.g., oils, cell cultures, feedstock, and environment materials (e.g., water, solvents, soil, air).

While methods and devices using sample material from a human have been described, biological sample materials may be derived from other sources. For example, the sample material may be derived from another mammal (e.g., a bovine (e.g., a cow), a porcine (e.g., a pig), a rodent (e.g., a mouse), or an equine (e.g., a horse). Typically, such samples are blood-derived samples such as plasma, serum, or whole blood. Sample material may also be derived from manufactured sources (e.g., cell cultures, solvents, oil, and food stuffs).

While methods and devices for determining albumin have been described, other analytes can be determined. For example, in some embodiments, the analyte includes a biological analyte (e.g., a protein (e.g., a protein found in blood), ischemia modified albumin, or combination thereof. Exemplary environmental applications of methods and devices described herein include, for example, human and animal diagnostics, the testing of environmental samples for contamination (e.g., by toxic agents, metals, toxins, bacteria, algae or the like). Food stuffs may be analyzed to ensure that there is not an unacceptable level of microbial, bacterial or viral, contamination present. Other fields where the methods and devices may be put to use, include, forensic science, aquaculture, veterinary, agriculture, food processing, and brewing.

While methods and devices that include determining an analyte (e.g., albumin) based on formation of complexes with cobalt have been described, first reagents other than cobalt can be used for determining albumin or other analytes. Examples of suitable first reagents include materials for which the analyte (e.g., albumin) has a different (e.g., higher) affinity than at least one other compound (e.g. IMA) in a mixture, for which the affinity with the analyte can be modified by another material (e.g., nickel), and which can be detected with different sensitivities when bound with the analyte (e.g., albumin) than when free of the analyte. For example, other metals (e.g., V, As, Co, Cu, Sb, Cr, Mo, Mn, Ba, Zn, Ni, Hg, Cd, Fe, Pb, Au and Ag) each form complexes with albumin more readily than with IMA. Such metals compete with other materials (e.g., with other metals (e.g., one another) or with antibodies) for albumin binding and can be detected (e.g., electrochemically) with different sensitivity when bound to albumin than when free of albumin. For example, Cu, Ni, Zn, Mn (Manganese), Co, and Mg, have a decreasing affinity for albumin (Cu having the highest affinity). In general, method 100 can be performed by optionally replacing cobalt with one of the other metals (e.g., Ni, Zn, Mn, or Mg) and optionally replacing Ni with one of the other metals having a higher affinity for albumin than the metal selected to replace cobalt. In an exemplary embodiment, method 100 is performed using cobalt and replacing nickel with copper.

Additional suitable materials include antibodies that have a different affinity for albumin than

In general, the determination step 120 includes determining a first reagent (e.g., cobalt) in a first mixture. The first reagent is typically a material that can be detected with a different (e.g., lower) sensitivity upon interaction with the analyte (e.g., when complexed with the analyte) than when such interaction is not present (e.g., when not complexed with the analyte). Thus, the determined amount of first reagent in the first mixture is typically less than the total amount of first reagent because first reagent that has interacted with the analyte is not detected and/or is detected with a lower sensitivity than first reagent that has not interacted with the analyte.

While methods and devices using a second reagent (e.g. nickel) to modify interaction between cobalt and albumin have been described, other second reagents may alternatively or additionally be used. In general, any material that modifies (e.g., reduces) interaction between cobalt and albumin can be used. Typical materials compete with cobalt for binding albumin and/or modify albumin so as to reduce its affinity for cobalt. For example, in some embodiments another metal (e.g., copper, a transition metal, or combination thereof) is used alone or in combination with nickel to modify interaction between cobalt and albumin. In an exemplary embodiment, the step 130 of forming a second mixture includes combining a portion of sample material with copper (e.g., as an aqueous solution (e.g., a copper sulfate solution) and/or a solid copper salt (e.g., copper sulfate)). Other suitable materials include biological materials (e.g., antibodies to albumin that block cobalt binding).

In general, the determination step 140 includes determining the first reagent in the presence of a second reagent. Typically, the second reagent and the analyte are capable of interacting to modify interaction between the first reagent and the analyte. For example, interaction between the second reagent and the analyte typically prevents, reduces, competes for, and/or disrupts interaction between the first reagent and the analyte. In general, the affinity of the second reagent for the analyte is higher than the affinity of the first reagent for the analyte.

The second reagent is typically a species capable of modifying (e.g., preventing and/or reducing) interaction between the first reagent and the analyte. In some embodiments, the second reagent displaces first reagent from a complex with the analyte.

In general, the second reagent is a species that has an affinity for the analyte that is higher than the affinity of the first reagent for the analyte. For example, in some embodiments, the analyte is a protein (e.g., albumin), the first reagent is a metal ion (e.g., cobalt) that complexes with the analyte, and the second reagent is a metal ion (e.g., nickel, copper, a transition metal, or combination thereof) that complexes with the analyte preferentially as compared to the first reagent.

While the first and second reagents may both be the same type of material (e.g., both reagents may be metal ions), these reagents may be different types of materials. For example, in some embodiments, the first reagent is a metal ion (e.g., cobalt, nickel, copper, transition metal, or combination thereof) and the second reagent is a different type of species (e.g., a biological compound such as an antibody for the analyte (e.g., an antibody for albumin), a protein, an enzyme, or other species that modifies the detectability of the first reagent).

In the determination step 140, first reagent in the second mixture is determined. Determination steps 120,140 are typically performed using the same detection technique. The amount of first reagent determined 140 in the second mixture is typically larger than the amount determined 120 in the first mixture because the second reagent modifies interaction between the first reagent and the analyte.

In a determining step 150, a value indicative of the amount of analyte present in the sample material is determined based at least on part on results of the determining steps 120, 140. Typically, the difference between the amount of first reagent determined 120 in the first mixture and the amount of first reagent determined 140 in the second mixture indicative of (e.g., is proportional to) the amount of analyte in the sample material. In some embodiments, the value indicative of the amount of analyte present in the sample material is determined based at least in part on the difference between the amount of first reagent determined 120 in the first mixture and the amount of first reagent determined 140 in the second mixture.

The first reagent is typically a species capable of interacting with the analyte (e.g., by forming a complex with the analyte). In some embodiments, the first reagent is a metal (e.g., a metal ion) such as cobalt.

While methods and devices including electrochemical determination of the amount of free first reagent (e.g., cobalt) have been described, other determination techniques can be used. For example, other electrochemical techniques including voltametry (e.g., stripping voltametry) and potentiometry can be used. Additionally, other electrode configurations (e.g., interdigitated electrodes) can be used.

Additional suitable determination techniques include optical techniques (e.g., fluorescence or absorption spectroscopy). In some embodiments, the first reagent is detected by colorimetry. For example, a detection zone of a microfluidic device may have one or more optical windows to allow light to enter and/or exit a device. The sample material is combined with a reagent that forms a colored complex in the presence of free cobalt but does not form a colored complex with cobalt complexed with albumin. The determination of free cobalt is made based on the optical density of the resulting mixture.

While devices that combine sample material with solid reagent (e.g., a salt) have been described, other configurations can be used. For example, any of the devices described herein can be configured with one or more reagent pouches that each includes a liquid reagent (e.g., an aqueous solution with reagent material to form first and second mixtures in accord with methods 100 or 400). The reagent pouches are typically connected to a microfluidic network by one or more channels. Reagent pouches on a device can also or alternatively include reagents that modify properties of the sample material to assist determination of the analyte. Exemplary reagents include buffers, lysing agents, pH modifiers, and diluents. In use, the reagent pouches are actuated (e.g., ruptured) allowing reagent therein to mix with sample material (see, e.g., U.S. Patent application No. 60/736,302, filed Nov. 5, 2005, which is incorporated by reference in its entirety).

While we have described methods and devices using detection techniques that have a different sensitivity for first reagent that has interacted with the analyte as compared to first reagent that has not interacted with the analyte, other detection techniques can be used. In some embodiments, the detection technique discriminates interacted first reagent from non-interacted first reagent. For example, the technique can be an electrochemical technique in which signals indicative of interacted and non-interacted first reagent appear at different potentials and/or currents. As another example, the detection technique can be an optical technique in which signals indicative of interacted and non-interacted first reagent appear at different wavelengths or have different lifetimes or polarizations.

### Examples

The following are non-limiting examples.

### Example 1

### Cobalt Amperometric Reduction Curves in Whole Blood

Whole blood from a human source and a cobalt reagent (CoCl₂) were combined to prepare a 1 mM Co solution in whole blood (a cobalt only solution). Whole blood from the same source, a cobalt reagent (CoCl₂), and a nickel reagent (NiCl₂) were combined to prepare a 1 mM Co/5 mM Ni solution in whole blood (a cobalt/nickel solution). Cobalt reduction currents were obtained from these solutions. It is believed that the whole blood was representative of a human source not having experienced an ischemic event.

Referring to FIG. 17, first and second cobalt reduction currents 600,602 are representative cobalt reduction currents obtained from the cobalt only solution. Third and fourth cobalt reduction currents 604,606 are representative cobalt reduction currents obtained from the cobalt/nickel solution. An electrochemical signal 608 is the difference between the negative peak 609 of the reduction current of curve 600 between 0.6 V and 0.8 V and the inflection point 611 of the curve, which occurs at a voltage between about 0.4 and 0.6 V. An electrochemical signal 610 is the difference between the negative peak 613 of the reduction current of curve 606 between 0.6 V and 0.8 V and the inflection point 615 of the curve, which occurs at a voltage between about 0.4 and 0.6 V.

Because of the presence of nickel in the solutions used to obtain curves 604,606, cobalt is displaced from albumin in the solutions. Thus, more cobalt is available as free cobalt to be detected amperometrically. Accordingly, electrochemical signal 610 from the cobalt/nickel solution has a greater magnitude than electrochemical signal 608 from the cobalt only solution.

### Example 2

### Cobalt Electrochemical Signals in the Presence of Nickel or Copper in Whole Blood

Whole blood from the human source used to prepare the solutions of Example 1 was combined with a cobalt reagent (CoCl₂) and a copper reagent (CuCl₂) to prepare multiple cobalt/copper solutions each being 1 mM Co and having a copper concentration ranging from 0 mM to 10 mM. Whole blood from the same human source was combined with a cobalt reagent (CoCl₂) and a nickel reagent (NiCl₂) to prepare multiple nickel/copper solutions each being 1 mM Co and having a copper concentration ranging from 0 mM to 20 mM. Cobalt reduction currents were obtained from each of the cobalt/copper and cobalt/nickel solutions.

Referring to FIG. 20, a current vs. Ni concentration curve 620 plots electrochemical signals from the cobalt/nickel solutions against Ni concentration. A current vs. Cu concentration curve 622 plots electrochemical signals from the cobalt/copper solutions against Cu concentration. The electrochemical signals are obtained as described in Example 1.

For the cobalt/nickel and cobalt/copper solutions, the magnitude of the cobalt electrochemical signal increases with increasing nickel or copper concentration because the amount of free cobalt increases. The electrochemical signals saturate (i.e., cease to increase) at a nickel or copper concentration of about 5 mM. This indicates that essentially all of the cobalt is available as free cobalt as opposed to cobalt bound by albumin.

### Example 3

### Cobalt Electrochemical Signal Calibration Curve in Whole Blood

Whole blood from the human source used to prepare the solutions of Example 1 was combined with a cobalt reagent (CoCl₂) to prepare multiple calibration solutions each having a cobalt concentration ranging from 1 mM Co to 3 mM Co. Nickel was not used. Electrochemical signals were obtained as described in Example 1.

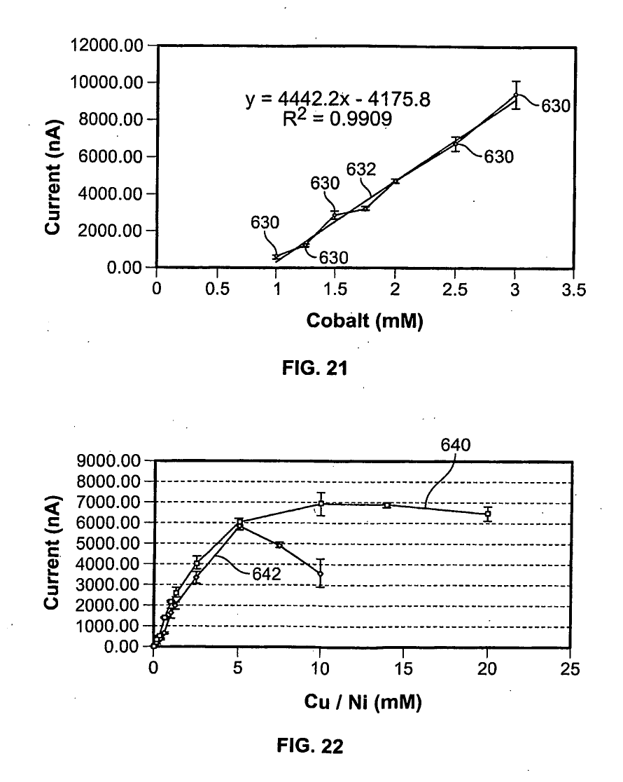

Referring to FIG. 21, electrochemical signals 630 obtained from the calibration solutions are plotted against cobalt concentration. A line 632 is the least-squares best fit to the electrochemical signals 630. Best fit line 632 has a negative intercept because albumin in the whole blood solution binds a portion of the cobalt. The amount of cobalt bound by the albumin can be estimated as about 0.94 mM based on the best fit slope and intercept.

### Example 4

### Cobalt Electrochemical Signals in the Presence of Nickel or Copper in Serum

Whole blood from the human source used to prepare the solutions of Example 1 was spun down to prepare plasma. The plasma was combined with a cobalt reagent (CoCl₂) and a copper reagent (CuCl₂) to prepare multiple cobalt/copper solutions each being 1.5 mM Co and having a copper concentration ranging from 0 mM to 10 mM. Another portion of the plasma was combined with a cobalt reagent (CoCl₂) and a nickel reagent (NiCl₂) to prepare multiple nickel/copper solutions each being 1.5 mM Co and having a copper concentration ranging from 0 mM to 20 mM. Cobalt reduction currents were obtained from each of the cobalt/copper and cobalt/nickel solutions.

Referring to FIG. 22, a current vs. Ni concentration curve 640 plots electrochemical signals from the cobalt/nickel solutions against Ni concentration. A current vs. Cu concentration curve 642 plots electrochemical signals from the cobalt/copper solutions against Cu concentration. The electrochemical signals are obtained as described in Example 1.

For the cobalt/nickel and cobalt/copper solutions, the magnitude of the cobalt electrochemical signal increases with increasing nickel or copper concentration because the amount of free cobalt increases. The electrochemical signals are substantially saturated (i.e., cease to increase) at a nickel or copper concentration of about 5 mM. This indicates that essentially all of the cobalt is available as free cobalt as opposed to cobalt bound by albumin.

### Example 5

### Cobalt Electrochemical Signal Calibration Curve in Serum

Serum from Example 4 was combined with a cobalt reagent (CoCl₂) to prepare multiple calibration solutions each having a cobalt concentration ranging from 1.5 mM Co to 4 mM Co (cobalt only solutions). Another portion of the serum was combined with a cobalt reagent (CoCl₂) and a nickel reagent (NiCl₂) to prepare multiple calibration solutions each having a cobalt concentration ranging from 1.5 mM Co to 4 mM Co and a nickel concentration of 20 mM (cobalt/nickel solutions).

Electrochemical signals were obtained as described in Example 1.

Referring to FIG. 23, electrochemical signals 650 obtained from the cobalt only calibration solutions are plotted against cobalt concentration. A line 652 is the least-squares best fit to the electrochemical signals 650. Best fit line 652 has a negative intercept because albumin in the whole blood solution binds a portion of the cobalt. The amount of cobalt bound by the albumin can be estimated as about 1.5 mM based on the best fit slope and intercept. Electrochemical signals 654 obtained from the cobalt/nickel calibration solutions are plotted against cobalt concentration. A line 656 is the least-squares best fit to the electrochemical signals 654.

### Example 6

### Effect of Nickel on Electrochemical Cobalt Signals in Whole Blood

Experiments were performed to study the effect of nickel concentration on the electrochemical (EC) determination of cobalt.

Blood was combined with an aqueous cobalt chloride solution and one of a series of aqueous nickel chloride solutions to form a series of mixtures each of which was 1.5 mM (actual, not apparent) in cobalt and ranged from 1 to 20 mM (actual, not apparent) in nickel. Blood from the same source was combined with a second aqueous cobalt chloride solution and one of the series of aqueous nickel chloride solutions to form a series of mixtures each of which was 2.5 mM (actual, not apparent) in cobalt and ranged from 1 to 20 mM (actual, not apparent) in nickel.

The solutions were introduced to an assay device using a pipette and amperometric determinations of cobalt were performed.

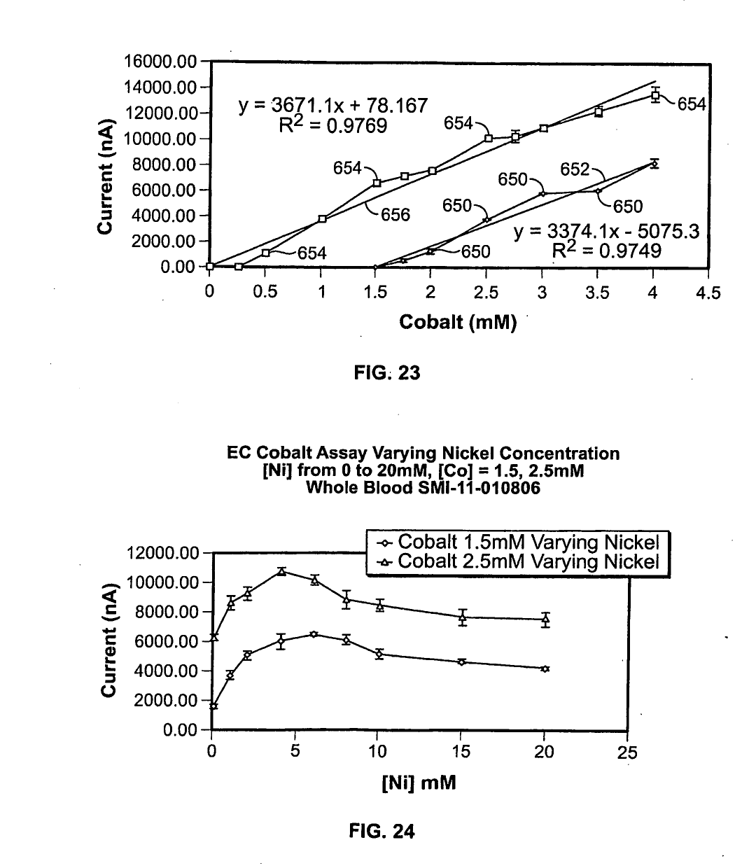

Referring to FIG. 24, the peak current response for both the 1.5 and 2.5 mM cobalt solutions increases with nickel concentration until a maximum is reached around 4 mM nickel. The cobalt response then declines, but less steeply between 10 and 20 mM. This behavior consistent with the idea that nickel competes with cobalt for binding albumin. The presence of nickel increases the level of “free” cobalt as the nickel concentration rises until the albumin becomes saturated with bound nickel. After the saturation point, adding more nickel does not further increase the cobalt signal. The fact that the nickel saturation concentration appears to be the same for both the

It is interesting to note the potential effect of hematocrit on the concentration of reagents which are added to whole blood because we believe that cobalt (and therefore probably nickel) is excluded from the red blood cells which causes the actual concentration of these metal ions in the surrounding plasma to be higher than anticipated. This particular blood sample had a hematocrit of 47%, which means that every millilitre of blood has only 0.53 ml of plasma to contain the cobalt and nickel. Their plasma concentrations may therefore be adjusted as follows: cobalt; 1.5 mM (actual)→2.8 mM (apparent); 2.5 mM→4.7 mM (apparent); nickel, 4 mM→7.5 mM (apparent); 20 mM→38 mM (apparent). This proposed “concentration” effect of hematocrit is strongly supported by a previous finding that a whole blood sample with a hematocrit of 49% had a nickel saturation point of 5 mM actual (with respect to the cobalt response), but this became 10 mM when the derived serum was also tested—a change which would be predicted by a hematocrit of this value.

To achieve an albumin-saturating nickel concentration of 7.5 mM in blood plasma, a blood with a 30% hematocrit would need to receive an amount of nickel sufficient for a 5.25 mM “apparent” concentration (this is the concentration which would be achieved if the red cells did not exclude nickel ions). The same amount of nickel would produce a 13 mM concentration in the plasma of a 60% hematocrit blood; the blood used in this work has a plasma nickel concentration of 13 mM at an apparent nickel concentration of 6.9 mM. There was relatively little difference in the cobalt signal between 4 and 6.9 mM nickel.

### Example 7

### Determination of Albumin Using Cobalt

Multiple human blood samples were obtained. A first subset of the samples were obtained by venous draw from healthy volunteers. Samples were either used directly as whole blood, or the samples were centrifuged to remove the red blood cells to produce either serum or plasma. A second subset of the samples (Ischemic samples) were obtained from an external supplier. Samples were supplied as frozen serum samples, which were aliquoted into 1 mL amounts for storage before use. The samples were obtained from patients suspected of undergoing an ischemic event. Each blood sample was divided into first and second 100 μL portions.

Each first 100 μL blood sample portion was treated as follows. The first 100 μL portion was combined with 5 μL of an aqueous cobalt chloride and potassium chloride solution to provide a mixture having a final concentration of 2.25 mM cobalt chloride and 75 mM potassium chloride. The mixture was incubated for 2 minutes. A first portion of the cobalt formed a complex with albumin in the mixture.

A 10 μL aliquot of the mixture was added to a detection zone of a test strip. The test strip included a detection zone defined by a polyester substrate and a polymer film. The detection zone included first and second screen-printed carbon electrodes and a silver/silver-chloride reference electrode.

The first working electrode was held at +1.0 Volts for 40 seconds. The first working electrode potential was scanned from +1.0 to −0.5 Volts at +0.7 Volts/second. The maximum negative current between +0.6 and +0.8 Volts was determined. Substantially all of the current in this potential range was due to cobalt that was not complexed with albumin.

A second aliquot of the mixture was subjected to an Albumin Cobalt Binding (ACB®) Test to optically determine the cobalt binding capacity of albumin present in the mixture. Only serum samples were analyzed using the ACB® test.

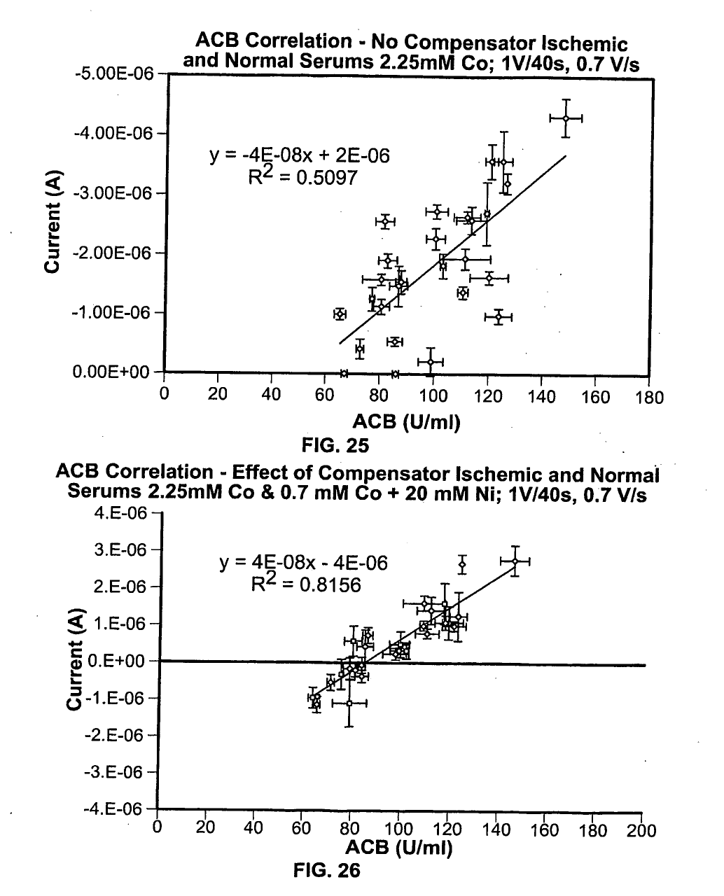

Referring to FIG. 25, the maximum negative current determined for the first 100 μL blood sample portion is plotted against cobalt binding capacity for that sample as determined by the (ACB®) test. In FIG. 26, the healthy samples define a first line offset from a line defined by the ischemic samples. The offset may result from differences in handling or concomitant species between the two sample types.

### Example 8

### Determination of Albumin Using Cobalt in the Presence of Nickel

Each second 100 μL blood sample portion from Example 7 was treated as follows. The second 100 μL blood sample portion was combined with 5 μL of an aqueous nickel chloride, cobalt chloride and potassium chloride solution to yield a solution having a 20 mM nickel concentration, a 0.7 mM cobalt concentration, and a 75 mM potassium chloride. The mixture was incubated for 2 minutes. Nickel preferentially (as compared to cobalt) formed a complex with albumin in the mixture. Essentially all of the cobalt remained uncomplexed with albumin.

A 10 μL aliquot of the mixture was added to a detection zone of a test strip as described in Example 1. The first working electrode was held at +1.0 Volts for 40 seconds. The first working electrode potential was scanned from +1.0 to −0.5 Volts at +0.7 Volts/second. The maximum negative current between +0.6 and +0.8 Volts was determined. Substantially all of the current in this potential range was due to cobalt that was not complexed with albumin.

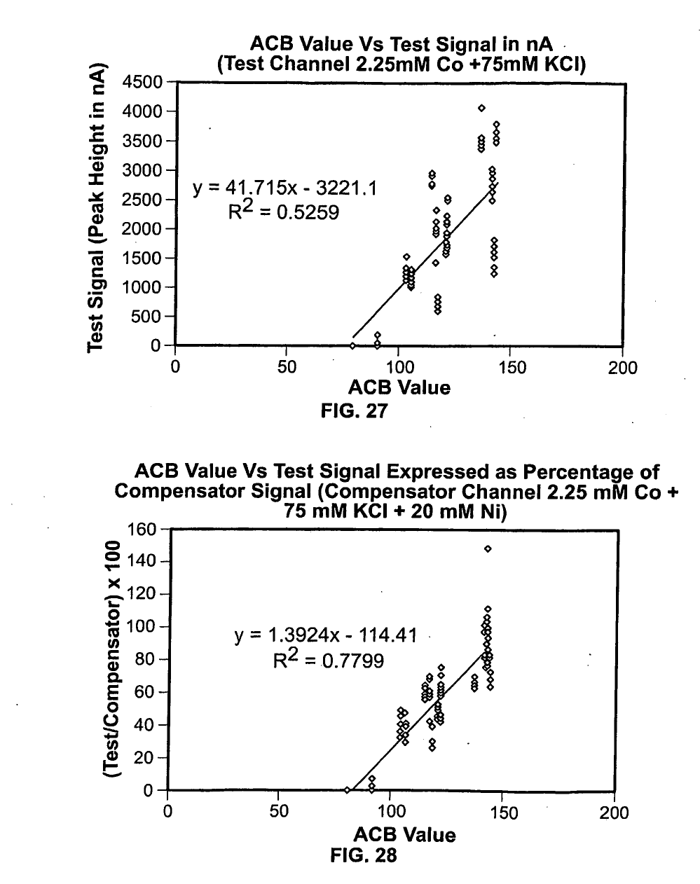

Referring to FIG. 27, the difference between the maximum negative current determined for the second 100 μL blood sample portion and the first 100 μL blood sample portion is plotted against cobalt binding capacity for that sample as determined by the (ACB®) test.

Comparing the data of FIGS. 24 and 25 it can be seen that the healthy samples define a line that overlaps a line defined by the ischemic samples. The use of nickel corrected for the current offset visible in FIG. 24. Addition, the precision of the data in FIG. 25 as determined from the quality of the straight line fits is better than the precision of the data in FIG. 24.

### Example 8

Samples were tested as described above; except that the cobalt concentration was constant at 2.25 mM in both cases (i.e., when tested in the absence or presence of added 20 mM nickel). FIG. 26 shows the peak height (in nA) for cobalt oxidation current plotted against the ACB value. FIG. 7 shows results for the samples when tested in the presence of 20 mM nickel. In FIG. 22, the ratio of the cobalt-only measurement to the cobalt-and-nickel measurement is plotted against the ACB value. As can be seen, the fit to a straight line is significantly better in FIG.22 than in FIG. 21.

### Example 9

A human subject experiencing (or who has recently experienced) an episode of chest pain or other cardiac symptoms is diagnosed by a medical professional. The professional determines that the chest pain or symptoms are of predominant cardiac origin. Following this determination, the subject blood-derived material from the subject is assayed on multiple occasions to determine whether the subject is experiencing (or has experienced) an ischemic event. For example, the subject may use an assay system described herein (e.g., an assay reader and assay devices described herein) to determine the presence of the ischemic event. The assays are performed on each of multiple occasions at intervals during a period of time following the determination. The intervals are typically at least about 4 hours and about 12 hours, for example between about 6 and about 8 hours. The period of time typically spans a high risk period of time for the subject in event that the chest pain was in fact of cardiac origin. For example, the measurements can be made for at least about 24 hours, for example at least about 48 hours, or at least about 72 hours.

### Example 10

Use of any of the methods and devices discussed herein to determine the presence of ischemia in patients who have had an intervention for their known heart disease. Since intervention (by-pass, stent) frequently causes complications and new ischemia or heart infarctions in the months after the surgery, monitoring for ischemia is helpful. The presence of ischemia is determined (e.g., in accord with method 100 or 400) on multiple occasions following an event or surgical intervention. Results from one or more of the multiple occasions are used to form a baseline. Subsequent results are compared to the baseline. For example, determinations can be made at least several times per week (e.g., daily or multiple times per day, e.g., at least twice per day). An increase in the level of IMA is indicative of ischemia or a probability of a cardiac event (e.g., a new heart attack).

### Example 11

A reagent suitable for use in methods and devices described herein was prepared. A mixture including 300 g water, 5 g MOPS buffer (Sigma M9027), 1.0 g Antifoam FDP (Basildon Chemicals), 0.75 g Tergitol 15-S-9, and 13.3 g Hydroxyethylcellulose (Natrosol G from Aqualon) was formed. The mixture was rolled over night to ensure that the Natrosol G is properly dissolved. 270 g of the solution was transferred to a new container. 8.0 g of silica powder (Cabosil TS610) was added and a Silverson mixer was used to disperse the silica evenly. 250 g of the mixture including the silica powder was transferred to a new container and 0.875 g of cobalt chloride was added to it.

### Example 12

The reagent material prepared in Example 11 was applied to electrodes of a device described herein. The reagent was applied by screen printing the reagent as an ink. The print was made in a rectangle which covered the working, reference, and counter electrodes of the device. A substrate was laminated over the electrodes to provide a detection zone having an internal height of 150 microns. No stir bar was used. The printed reagent was allowed to dry. Multiple devices were prepared.

The devices prepared above were tested using blood which spiked with different nickel concentrations to mimic blood with different cobalt binding abilities. The blood was added to the detection zone and allowed to form a mixture with the reagent material without agitation or other assisted mixing.

The amount of free cobalt in each mixture was determined using amperometry as described herein.

For comparison purposes, an assay was performed by combining 5 microliters of an aqueous cobalt chloride solution and 95 microliters of blood to give a final cobalt concentration in the blood of 1.5 mM. This mixture was added to devices identical to those having the screen printed reagent except for the absence of the reagent. The amount of free cobalt was determined using amperometry.

The amount of free cobalt in the mixtures formed using a printed reagent was similar to the amount formed using the aqueous cobalt reagent, which demonstrated that the cobalt of the printed reagent had resuspended and bound with albumin in the sample material even without assisted mixing.

Other embodiments are within the scope of the following claims.

1. An assay device comprising:
means to form a first mixture comprising a cobalt reagent and a blood derived sample material,
means to determine an amount or concentration of free cobalt in the first mixture,
means to form a second mixture comprising a cobalt reagent, blood derived sample material, and optionally a nickel reagent,
wherein if no nickel reagent is contained in the second mixture, then the amount of cobalt in the second mixture is different from the amount of cobalt in the first mixture, and
means to determine an amount or concentration of free cobalt in the second mixture.

2. The assay device of claim 1, wherein the means to form a second mixture comprises a nickel reagent.

3. The assay device of claim 2, wherein the nickel reagent disposed in a dry state within the device.

4. The assay device of claim 1, wherein the means to form a first mixture comprises a cobalt reagent disposed in a dry state within the device.

5. An assay device comprising:
a first detection zone and a second detection zone,
a first reagent material comprising a first metal, and
a second reagent material comprising either the first metal and a second metal having a higher affinity for albumin than the first metal, or comprising a different amount of the first metal;
wherein:
the device is configured to receive sample liquid and form a first mixture in the first detection zone, the first mixture comprising a portion of the sample liquid and the first reagent material, and to form a second mixture in the second detection zone, the second mixture comprising a portion of the sample liquid and the second reagent material.

6. The assay device according to claim 5, wherein said first and second detection zones are electrochemical detection zones.

7. The assay device according to claim 5, wherein said first metal is cobalt.

8. The assay device according to claim 5, wherein said second metal is nickel.

9. The device of claim 5, wherein the first metal is selected from the group consisting of V, As, Co, Cu, Sb, Cr, Mo, Mn, Ba, Zn, Ni, Hg, Cd, Fe, Pb, Au and Ag.

10. The method of claim 5, wherein the second metal is selected from the group consisting of V, As, Co, Cu, Sb, Cr, Mo, Mn, Ba, Zn, Ni, Hg, Cd, Fe, Pb, Au and Ag.

11. The assay device of claim 5, wherein at least one of said first and said second metal is present in the dry state before application of sample liquid.

12. The assay device of claim 5, wherein said sample liquid is selected from human blood and human blood plasma.

13. The assay device of claim 5, wherein the first or second metal is in the form of a salt of the metal.

14. A method for determining the presence of ischemia modified albumin in a sample derived from human blood, comprising:
forming a first mixture adding a part of said blood derived sample material to a cobalt reagent,
determining an amount or concentration of free cobalt in the first mixture to obtain a first result,
forming a second mixture by adding another part of said blood derived sample material to a cobalt reagent and optionally an amount of a nickel reagent sufficient to substantially prevent the formation and presence of cobalt albumin complexes in said part of the blood derived sample material,
wherein if no nickel reagent is contained in the second mixture, then the amount of cobalt in the second mixture is different from the amount of cobalt in the first mixture,
determining an amount or concentration of free cobalt in the second mixture to obtain a second result, and
processing the first and the second results and comparing the processed value with a suitable reference value that is indicative of ischemia-modified albumin.

15. The method of claim 14 wherein the reference value is indicative of the amount or concentration of free cobalt in a sample derived from blood of a human known to contain ischemic modified albumin following an ischemic event.

16. The method of claim 14 wherein the reference value is indicative of the amount or concentration of free cobalt in a sample derived from blood of the same human taken earlier.

17. A method for determining the presence of ischemia modified albumin in a sample derived from human blood, comprising:
forming a first mixture adding a part of said blood derived sample material to a first reagent comprising a first metal,
determining an amount or concentration of free first metal in the first mixture to obtain a first result,
forming a second mixture by adding another part of said blood derived sample material to an amount of the first reagent and optionally an amount of a second reagent comprising a second metal having a higher affinity for albumin than the first metal, the amount of second metal being sufficient to substantially prevent the formation and presence of first metal-albumin complexes in said part of the blood derived sample material,
wherein if no second reagent is contained in the second mixture, then the amount of first metal in the second mixture is different from the amount of first metal in the first mixture,
determining an amount or concentration of free first metal in the second mixture to obtain a second result, and
processing the first and the second results and comparing the processed value with a suitable reference value that is indicative of ischemia-modified albumin.

18. The method of claim 17, wherein the first metal is selected from the group consisting of V, As, Co, Cu, Sb, Cr, Mo, Mn, Ba, Zn, Ni, Hg, Cd, Fe, Pb, Au and Ag.

19. The method of claim 18, wherein the second metal is selected from the group consisting of V, As, Co, Cu, Sb, Cr, Mo, Mn, Ba, Zn, Ni, Hg, Cd, Fe, Pb, Au and Ag.

20. An assay system, comprising:
an assay device comprising first and second electrochemical detection zones each comprising a mixture of blood derived sample material and a cobalt reagent, the sample material having been obtained from a mammal, and
an assay reader configured to operate the assay device to determine a respective amount of free cobalt in the first and second detection zones and determine the presence of an ischemic event in the mammal based on the amounts of free cobalt.
